SEQUENCE LISTING (83 sequences)

SEQ ID NO: 1 - protein, 114 aa, Homo sapiens
MASVSELACIYSALILHDDEVTVTEDKINALIKAAGVNVEPFWPGLFAKALANVNIGSLICNVGAGGPAPAAGAAPAGGPAPSTAAAPAEEKKVEAKKEESEESDDDMGFGLFD

SEQ ID NO: 2 - DNA, 20 bp, synthetic construct
  source 1..20: mol_type = other DNA
tctctaaggaaggccgcata

SEQ ID NO: 3 - DNA, 20 bp, synthetic construct
  source 1..20: mol_type = other DNA
gatcacatcgattggctcct

SEQ ID NO: 4 - DNA, 22 bp, synthetic construct
  source 1..22: mol_type = other DNA
gttggaagaggaagtcagggca

SEQ ID NO: 5 - DNA, 20 bp, synthetic construct
  source 1..20: mol_type = other DNA
ccgtcttcagagagccaatc

SEQ ID NO: 6 - DNA, 19 bp, synthetic construct
  source 1..19: mol_type = other DNA
aagccccggggaccttcag

SEQ ID NO: 7 - DNA, 19 bp, synthetic construct
  source 1..19: mol_type = other DNA
cccgggagcaggtgacctt

SEQ ID NO: 8 - DNA, 20 bp, synthetic construct
  source 1..20: mol_type = other DNA
gtcgatgttggtgcttctca

SEQ ID NO: 9 - DNA, 18 bp, synthetic construct
  source 1..18: mol_type = other DNA
ggctcccagcaccatgaa

SEQ ID NO: 10 - DNA, 20 bp, synthetic construct
  source 1..20: mol_type = other DNA
acctcctggaacggctaaat

SEQ ID NO: 11 - DNA, 18 bp, synthetic construct
  source 1..18: mol_type = other DNA
acctggttgatcctgcca

SEQ ID NO: 12 - DNA, 20 bp, synthetic construct
  source 1..20: mol_type = other DNA
atcactctgcccaccatcac

SEQ ID NO: 13 - DNA, 20 bp, synthetic construct
  source 1..20: mol_type = other DNA
ttgcttctcggcttacctgt

SEQ ID NO: 14 - DNA, 20 bp, synthetic construct
  source 1..20: mol_type = other DNA
gacctttcccgaatggaacc

SEQ ID NO: 15 - DNA, 20 bp, synthetic construct
  source 1..20: mol_type = other DNA
ggctgttctacgctgcctac

SEQ ID NO: 16 - DNA, 20 bp, synthetic construct
  source 1..20: mol_type = other DNA
tgctgtctgtcctttgcatc

SEQ ID NO: 17 - DNA, 20 bp, synthetic construct
  source 1..20: mol_type = other DNA
tcaagctcatttggatgctg

SEQ ID NO: 18 - DNA, 20 bp, synthetic construct
  source 1..20: mol_type = other DNA
ctggggaacttccatcaaga

SEQ ID NO: 19 - DNA, 20 bp, synthetic construct
  source 1..20: mol_type = other DNA
caatttcatcatcgccctct

SEQ ID NO: 20 - DNA, 20 bp, synthetic construct
  source 1..20: mol_type = other DNA
cagtctgacatcctggctca

SEQ ID NO: 21 - DNA, 20 bp, synthetic construct
  source 1..20: mol_type = other DNA
agcgtggcattgtggatcag

SEQ ID NO: 22 - DNA, 23 bp, synthetic construct
  source 1..23: mol_type = other DNA
cagcacctttgtggttctcactt

SEQ ID NO: 23 - DNA, 20 bp, synthetic construct
  source 1..20: mol_type = other DNA
gtcgatgttggtgcttctca

SEQ ID NO: 24 - DNA, 20 bp, synthetic construct
  source 1..20: mol_type = other DNA
agccatgtacgtagccatcc

SEQ ID NO: 25 - DNA, 20 bp, synthetic construct
  source 1..20: mol_type = other DNA
ccatccttctcccagatcaa

SEQ ID NO: 26 - DNA, 20 bp, synthetic construct
  source 1..20: mol_type = other DNA
cattcgaacgtctgccctat

SEQ ID NO: 27 - DNA, 20 bp, synthetic construct
  source 1..20: mol_type = other DNA
ttcagcaaggaggaggtcat

SEQ ID NO: 28 - DNA, 20 bp, synthetic construct
  source 1..20: mol_type = other DNA
cttcagctgcctcaacacag

SEQ ID NO: 29 - DNA, 20 bp, synthetic construct
  source 1..20: mol_type = other DNA
gaggacccgtacagcctaca

SEQ ID NO: 30 - DNA, 20 bp, synthetic construct
  source 1..20: mol_type = other DNA
gaggagtatgcactgcgtga

SEQ ID NO: 31 - DNA, 22 bp, synthetic construct
  source 1..22: mol_type = other DNA
cctgtggttgtcatcgaagcct

SEQ ID NO: 32 - DNA, 23 bp, synthetic construct
  source 1..23: mol_type = other DNA
gcctctaaagatggtcgcattcg

SEQ ID NO: 33 - DNA, 23 bp, synthetic construct
  source 1..23: mol_type = other DNA
ctacatcagtggacttgcacctc

SEQ ID NO: 34 - DNA, 22 bp, synthetic construct
  source 1..22: mol_type = other DNA
atgtcagtgggacttgtgctgc

SEQ ID NO: 35 - DNA, 22 bp, synthetic construct
  source 1..22: mol_type = other DNA
ttgcaggcttctccaactggac

SEQ ID NO: 36 - DNA, 22 bp, synthetic construct
  source 1..22: mol_type = other DNA
agatggcagtggtgtcaaggag

SEQ ID NO: 37 - DNA, 20 bp, synthetic construct
  source 1..20: mol_type = other DNA
tgtccttctgccatggccct

SEQ ID NO: 38 - DNA, 21 bp, synthetic construct
  source 1..21: mol_type = other DNA
gatcgaacgctctgtggcagg

SEQ ID NO: 39 - DNA, 22 bp, synthetic construct
  source 1..22: mol_type = other DNA
cactcaagaggctgacgcagaa

SEQ ID NO: 40 - DNA, 22 bp, synthetic construct
  source 1..22: mol_type = other DNA
gcagaatccacgccagtacaag

SEQ ID NO: 41 - DNA, 22 bp, synthetic construct
  source 1..22: mol_type = other DNA
ggcaggagaatgtgctggtcat

SEQ ID NO: 42 - DNA, 20 bp, synthetic construct
  source 1..20: mol_type = other DNA
gatgcaaaggacagacagca

SEQ ID NO: 43 - DNA, 20 bp, synthetic construct
  source 1..20: mol_type = other DNA
ttgtggtggaggaaaaggtc

SEQ ID NO: 44 - DNA, 20 bp, synthetic construct
  source 1..20: mol_type = other DNA
tgcagccatgtcgtactctc

SEQ ID NO: 45 - DNA, 22 bp, synthetic construct
  source 1..22: mol_type = other DNA
atcacggagaccttctgctcag

SEQ ID NO: 46 - DNA, 20 bp, synthetic construct
  source 1..20: mol_type = other DNA
gaaatggactcgtcggtgtt

SEQ ID NO: 47 - DNA, 23 bp, synthetic construct
  source 1..23: mol_type = other DNA
tctccagctggtagagggagcag

SEQ ID NO: 48 - DNA, 23 bp, synthetic construct
  source 1..23: mol_type = other DNA
tctccagctggtagagggagcag

SEQ ID NO: 49 - DNA, 20 bp, synthetic construct
  source 1..20: mol_type = other DNA
aagcagcactcttccacgat

SEQ ID NO: 50 - DNA, 20 bp, synthetic construct
  source 1..20: mol_type = other DNA
gccaccgatccacagagagt

SEQ ID NO: 51 - DNA, 20 bp, synthetic construct
  source 1..20: mol_type = other DNA
ttgatctgggagaaggatgg

SEQ ID NO: 52 - DNA, 21 bp, synthetic construct
  source 1..21: mol_type = other DNA
gccattcgcagtttcactgta

SEQ ID NO: 53 - DNA, 20 bp, synthetic construct
  source 1..20: mol_type = other DNA
tgacctcctccttgctgaag

SEQ ID NO: 54 - DNA, 20 bp, synthetic construct
  source 1..20: mol_type = other DNA
tgtgcacgtcacagagatga

SEQ ID NO: 55 - DNA, 19 bp, synthetic construct
  source 1..19: mol_type = other DNA
gttccgctgtgtaagcacc

SEQ ID NO: 56 - DNA, 19 bp, synthetic construct
  source 1..19: mol_type = other DNA
aggtcccttcttgcatcct

SEQ ID NO: 57 - DNA, 20 bp, synthetic construct
  source 1..20: mol_type = other DNA
ttgtgcatccttgtggttgt

SEQ ID NO: 58 - DNA, 20 bp, synthetic construct
  source 1..20: mol_type = other DNA
agatgtcatggccaaccatt

SEQ ID NO: 59 - DNA, 20 bp, synthetic construct
  source 1..20: mol_type = other DNA
agtctctggaagtggctgga

SEQ ID NO: 60 - DNA, 20 bp, synthetic construct
  source 1..20: mol_type = other DNA
tgcagcaatttcgtcaaaag

SEQ ID NO: 61 - DNA, 20 bp, synthetic construct
  source 1..20: mol_type = other DNA
agatgctggcaatagggttg

SEQ ID NO: 62 - DNA, 25 bp, synthetic construct
  source 1..25: mol_type = other DNA
tcaaaggctttattcattgcagagg

SEQ ID NO: 63 - DNA, 20 bp, synthetic construct
  source 1..20: mol_type = other DNA
ctccacccagctccagttgt

SEQ ID NO: 64 - DNA, 20 bp, synthetic construct
  source 1..20: mol_type = other DNA
aagcagcactcttccacgat

SEQ ID NO: 65 - DNA, 20 bp, synthetic construct
  source 1..20: mol_type = other DNA
ctctcagctgtggtggtgaa

SEQ ID NO: 66 - DNA, 20 bp, synthetic construct
  source 1..20: mol_type = other DNA
cttgggctgtagcagtctcc

SEQ ID NO: 67 - DNA, 20 bp, synthetic construct
  source 1..20: mol_type = other DNA
gtttctcaggctccctctcc

SEQ ID NO: 68 - DNA, 20 bp, synthetic construct
  source 1..20: mol_type = other DNA
ccgccaacttctcgtatttc

SEQ ID NO: 69 - DNA, 20 bp, synthetic construct
  source 1..20: mol_type = other DNA
gctgtgcttgggattggtat

SEQ ID NO: 70 - DNA, 20 bp, synthetic construct
  source 1..20: mol_type = other DNA
cgttgtcccgctactacgtt

SEQ ID NO: 71 - DNA, 20 bp, synthetic construct
  source 1..20: mol_type = other DNA
gtgaagtgggcatttcaggt

SEQ ID NO: 72 - DNA, 22 bp, synthetic construct
  source 1..22: mol_type = other DNA
ctcacatgccaggtacactagc

SEQ ID NO: 73 - DNA, 22 bp, synthetic construct
  source 1..22: mol_type = other DNA
aaactccacgctgctgtccagt

SEQ ID NO: 74 - DNA, 23 bp, synthetic construct
  source 1..23: mol_type = other DNA
cagaaagtggctggatgatgtcc

SEQ ID NO: 75 - DNA, 22 bp, synthetic construct
  source 1..22: mol_type = other DNA
aactcagccaccatgaaccagg

SEQ ID NO: 76 - DNA, 22 bp, synthetic construct
  source 1..22: mol_type = other DNA
cagaaccaggagcacagtgaag

SEQ ID NO: 77 - DNA, 22 bp, synthetic construct
  source 1..22: mol_type = other DNA
ctggatggcatactggaagcag

SEQ ID NO: 78 - DNA, 20 bp, synthetic construct
  source 1..20: mol_type = other DNA
ttcacaaaggctgcggctgg

SEQ ID NO: 79 - DNA, 22 bp, synthetic construct
  source 1..22: mol_type = other DNA
gctctgaggacccttggagaaa

SEQ ID NO: 80 - DNA, 22 bp, synthetic construct
  source 1..22: mol_type = other DNA
ggttgatctgggagaaggatgg

SEQ ID NO: 81 - DNA, 22 bp, synthetic construct
  source 1..22: mol_type = other DNA
gcttgttgtccagaccattggc

SEQ ID NO: 82 - DNA, 23 bp, synthetic construct
  source 1..23: mol_type = other DNA
cataagcgacaggcgtcagtttc

SEQ ID NO: 83 - DNA, 20 bp, synthetic construct
  source 1..20: mol_type = other DNA
actgatgcggctcatctctt

(19) United States
(12) Patent Application Publication
(10) Pub. No.: US 2026/0295074 A1
(43) Pub. Date: Oct. 1, 2026

(54) Ribosomal Remodeling in Diabetes

(71) Applicant: Joslin Diabetes Center, Inc., Boston, MA (US)

(72) Inventors: Jean Schaffer, Boston, MA (US); Abigael Cheruiyot, Brighton, MA (US)

(73) Assignee: Joslin Diabetes Center, Inc., Boston, MA (US)

(21) Appl. No.: 19/465,189

(22) Filed: Jan. 30, 2026

Related U.S. Application Data
(63) Continuation of application No. PCT/US24/40625, filed on Aug. 1, 2024.
(60) Provisional application No. 63/517,267, filed on Aug. 2, 2023.

(51) Int. Cl. A61K 48/00; A61K 35/39; A61K 35/545; A61K 45/06; A61P 3/10; C07K 14/62; C12N 5/071; C12N 5/074; C12N 15/10

(52) U.S. Cl. CPC A61K 48/00; A61K 35/39; A61K 35/545; A61K 45/06; A61P 3/10; C07K 14/62; C12N 5/0676; C12N 5/0696; C12N 15/102

(57) ABSTRACT

This disclosure relates to ribosomal remodeling due to high glucose conditions. In some embodiments, translation of proteins in pancreatic beta-cells is altered based on the ribosomal remodeling. Means of increasing the percentage of large ribosomal subunit protein P1 (RPLP1) associated with ribosomes and/or decreasing the percentage of soluble RPLP1 in a cell to counteract effects of high glucose conditions in diabetes are disclosed.

### CROSS-REFERENCE TO RELATED APPLICATIONS

This application is a continuation of International Patent Application No. PCT/US2024/040625, filed on Aug. 1, 2024, which claims the benefit of U.S. Provisional Patent Application No. 63/517,267, filed Aug. 2, 2023. The entire contents of the foregoing applications are incorporated herein by reference in their entirety including all text, tables and drawings.

### SUPPORT

This invention was made with government support under Research Grant No. 5DP1 DK119141 awarded by NIH/National Institute of Diabetes and Digestive and Kidney Diseases. The government has certain rights in the invention.

### REFERENCE TO SEQUENCE LISTING

The present application contains a Sequence Listing which has been submitted electronically in XML format and is hereby incorporated by reference in its entirety. Said XML copy created on Jul. 25, 2024, is named “01123-0015-00PCT-ST26.xml” and is 74,137 bytes in size.

### FIELD

This disclosure relates to ribosomal remodeling due to high glucose conditions. In some embodiments, translation of proteins in pancreatic beta-cells is altered based on the ribosomal remodeling. Means of increasing the percentage of Large Ribosomal Subunit Protein P1 (RPLP1) associated with ribosomes and/or decreasing the percentage of soluble RPLP1 in a cell to counteract effects of high glucose conditions in diabetes are disclosed.

### BACKGROUND

Diabetes results from pancreatic beta-cell failure to secrete sufficient insulin to regulate glucose homeostasis. Progressive decline in beta-cell function occurs in the setting of hyperglycemia during the progression from early appearance of autoantibodies to frank type 1 diabetes, and during the evolution from compensated insulin resistance to type 2 diabetes (1, 2). In type 1 diabetes, intensive insulin therapy that restores normal glycemic levels increases stimulated C-peptide levels, a reflection of improved insulin biosynthesis and preserved beta-cell function (3). In patients with newly diagnosed type 2 diabetes, intensive short-term insulin therapy improves beta-cell function and long-term glycemic control (4, 5). These observations support the hypothesis that glucose toxicity contributes to the decline in insulin production in diabetes.

Pancreatic beta-cells are specialized for coupling glucose metabolism to insulin peptide production and secretion. Acute glucose exposure robustly and coordinately increases translation of proinsulin and proteins required for secretion of mature insulin peptide. By contrast, chronically elevated glucose levels that occur during diabetes impair beta-cell insulin secretion and have been shown experimentally to suppress insulin translation. Whether translation of other genes critical for insulin secretion are similarly downregulated by chronic high glucose is unknown.

In response to an acute physiological rise in glucose, proinsulin mRNA translation rapidly increases within 30 to 60 minutes, without change in insulin mRNA abundance (6). This translational regulation requires sequences predicted to form a stem-loop structure within the 5′-untranslated region of the insulin mRNA that bind to protein factors in a glucose-dependent fashion (7). In addition to insulin, glucose acutely upregulates translation of proteins involved in glucose metabolism, insulin processing, secretory granule biogenesis, and insulin exocytosis, without causing an equivalent increase in total protein synthesis (8-11). Given that newly synthesized insulin is preferentially released initially, this mRNA translational program supports physiological increases in glucose-stimulated insulin secretion (GSIS) following brief exposures to high glucose (12).

By contrast, persistently elevated glucose impairs GSIS and insulin translation. Among obese subjects, GSIS decreases with increasing plasma glucose area under the curve in a 3-hour oral glucose tolerance test, without change in insulin sensitivity (13). Ex vivo incubation of isolated human or rodent islets over one week in media containing high glucose also impairs GSIS and inhibits translation of proinsulin (14, 15). The impact of sustained high glucose on GSIS in cadaveric human islets is apparent as early as 2 days following exposure to high glucose, a time point at which impairment is reversible, associated with only modest transcriptomic changes, and without evidence for ER stress (16). Sustained exposure of insulinoma cells and isolated islets to high concentrations of glucose and saturated fatty acids to model the metabolic stress of type 2 diabetes increases translation of JUND, a transcriptional regulator of beta-cell apoptosis, and induces ER stress (17, 18). However, it is not known whether sustained high glucose alone initiates programmatic regulation of translation prior to induction of ER stress.

Given the importance of translational regulation in GSIS, the impact of sustained glucose elevation was studied on genome-wide beta-cell mRNA translation. Using complementary high-throughput approaches in a MIN6 model and validating findings in primary isolated islets ex vivo and in an in vivo model of hyperglycemia, chronic glucose excess was shown to coordinately downregulate translation of genes that function in metabolism-coupled insulin secretion.

The present disclosure adds to a growing body of evidence that the mammalian proteome is strongly influenced by mRNA-specific rates of translation (40), a process that can rapidly regulate protein abundance independent of new transcription or RNA decay.

Selective mRNA translation often leverages unique features within untranslated regions of mRNAs, trans-acting mRNA binding proteins, and/or selection of specific translation initiation or elongation factors (50). Although the widely held dogma is that all ribosomes are compositionally and functionally similar (51), growing evidence supports the idea that ribosomes with varying stoichiometry of select ribosomal proteins, known as heterogeneous ribosomes, direct functionally distinct programs of mRNA translation (52-56). To date, examples of heterogeneous ribosomes have been identified as stable features of distinct cell types or tissues and are presumed to be generated during ribosome biogenesis in the nucleolus.

Disclosed herein is the impact of sustained high glucose on the composition of actively translating ribosomes (polysomes) in beta-cells. As described in this disclosure, RPLP1 dissociates from ribosomes of high glucose-treated cells. Thus, exposure of pancreatic beta-cells to sustained high glucose, not only decreases translation of key genes for beta-cell function but also dynamically remodels actively translating ribosomes. RPLP1 is known to be phosphorylated on serine residues 101 and 104 (Sacco F, et al. (2016) Nat Commun. 7:13250). As described in this disclosure, mutation of serine 101 and serine 104 to alanine, which renders them non-phosphorylatable, decreases RPLP1 association with translating ribosomes. Methods disclosed herein can be used to increase association of RPLP1 in ribosomes and/or to decrease soluble RPLP1 outside of ribosomes to prevent maladaptive changes in mRNA translation.

Targeted ribosome remodeling for therapeutic effect on protein expression could also be applied to diabetes as well as other disorders. This approach could therefore serve as a platform technology to address many human diseases.

### SUMMARY

High-throughput ribosome profiling and nascent proteomics in MIN6 insulinoma cells as described herein elucidate the genome-wide impact of sustained high glucose on beta-cell mRNA translation. Prior to induction of ER stress or suppression of global translation, sustained high glucose suppressed glucose-stimulated insulin secretion and downregulated translation of not only insulin but also genes related to insulin secretory granule formation, exocytosis, and metabolism-coupled insulin secretion. Translation of these genes was also downregulated in primary rat and human islets following ex-vivo incubation with sustained high glucose and in an in vivo model of chronic mild hyperglycemia. Furthermore, translational downregulation decreased cellular abundance of these proteins.

In some embodiments, a translational regulatory circuit during beta-cell glucose toxicity impairs expression of proteins with critical roles in beta-cell function, and the present methods inhibit this circuit.

As described herein, interventions to increase large ribosomal subunit protein P1 (RPLP1) association with ribosomes could be used to improve production of key proteins in beta-cell insulin secretion. Such a strategy could be used to enhance insulin secretion in vivo during the development or progression of type 1 or type 2 diabetes. In some embodiments, treatments to increase RPLP1 association with ribosomes improves the function of beta-cells or iPSC-generated beta-like cells used for cell replacement therapies. In some embodiments, modification of these cells to increase RPLP1 phosphorylation prior to implantation increases glucose-stimulated secretion of insulin. Exemplary embodiments are listed below:

Embodiment 1. A method of treating or preventing diabetes in a subject in need thereof comprising increasing the percentage of, ribosome-associated RPLP1 or decreasing the percentage of soluble RPLP1 in a cell of the subject comprising phosphorylating RPLP1, inhibiting dephosphorylation of RPLP1, and/or expressing an RPLP1 mutant comprising one or more phosphomimetic amino acids, thereby increasing the percentage of ribosome-associated RPLP1 and/or decreasing the percentage of soluble RPLP1 in the subject.

Embodiment 2. The method of embodiment 1, wherein the subject has Type I (T1) or Type II (T2) diabetes.

Embodiment 3. The method of embodiment 1, wherein the subject is at risk for T2 diabetes. For example, in some cases the subject may have pre-diabetes such as an HbA1c level of 5.8-6.5% or 5.8-6.4%, or may have a family history of T1 or T2 diabetes.

Embodiment 4. The method of any one of embodiments 1-3, wherein increasing the percentage of ribosomes containing RPLP1 increases translation of one or more proteins encoded by a gene selected from INS, SCGN, IDH2, VPS41, SLC2A1, IGF2, SLC30A8, and PFKFB3.

Embodiment 5. The method of any one of embodiments 1-3, wherein the method comprises administering to the subject a kinase activator or phosphatase inhibitor that phosphorylates RPLP1 and/or dephosphorylates RPLP1.

Embodiment 6. The method of any one of embodiments 1-3, wherein the RPLP1 phosphorylation is at serine 101 and/or serine 104.

Embodiment 7. The method of any one of embodiments 1-3, wherein expressing an RPLP1 mutant comprising one or more phosphomimetic amino acids is performed by gene editing of RPLP1.

Embodiment 8. The method of embodiment 7, wherein the gene editing is performed with a system for gene editing.

Embodiment 9. The method of embodiment 8, wherein the system for gene editing comprises a CRISPR/Cas9 system, zinc-finger nuclease, transcription activator-like effector nuclease (TALEN), meganuclease, or group one intron encoded endonuclease (GIIEE).

Embodiment 10. The method of any one of embodiments 7-9, wherein the RPLP1 mutant comprises one or more phosphomimetic amino acid mutations at an amino acid that is a serine in wildtype RPLP1.

Embodiment 11. The method of embodiment 10, wherein the serine in wildtype RPLP1 is serine 101 and/or serine 104.

Embodiment 12. The method of any one of embodiments 1-4 or 7-11, wherein the one or more phosphomimetic amino acid mutation is an aspartic acid or glutamic acid.

Embodiment 13. The method of any one of embodiments 1-12, wherein the cell is a pancreatic beta-cell.

Embodiment 14. The method of any one of embodiments 1-13, wherein the method increases the levels of insulin within or released by the cell.

Embodiment 15. The method of any one of embodiments 5, 6, or 14, wherein the cell is a pancreatic beta-cell, and the subject is treated with a kinase activator or phosphatase inhibitor.

Embodiment 16. The method of any one of embodiments 1-4 or 7-15, wherein the cell is an induced pluripotent stem cell (iPSC) or pancreatic beta-cell in culture and gene editing is performed in vitro, and the cell is introduced into the subject after the gene editing or where the cell is an iPSC or pancreatic beta-cell in a subject and gene editing is performed in vivo using a delivery system that selectively delivers the gene editing system to the iPSC or pancreatic beta-cell.

Embodiment 17. The method of embodiment 15 or embodiment 16, wherein insulin levels in the subject are increased and/or glucose levels in the subject are decreased.

Embodiment 18. A method of treating or preventing diabetes in a subject in need thereof comprising preparing iPSCs and/or pancreatic beta-cells in vitro; treating said iPSCs or pancreatic beta-cells with an agent for gene editing of RPLP1, wherein the gene editing causes expression of an RPLP1 mutant comprising one or more phosphomimetic amino acids in the cell; and transplanting the treated iPSCs or pancreatic beta-cells into the subject, wherein the transplanting increases insulin levels in the subject and/or decreases glucose levels in the subject. In some cases, the subject has T1 or T2 diabetes, or alternatively is at risk for T2 diabetes. For example, in some cases the subject may have pre-diabetes such as an HbA1c level of 5.8-6.5% or 5.8-6.4%, or may have a family history of T1 or T2 diabetes.

Embodiment 19. A method of reducing the incidence of hyperglycemia or immune attack on pancreatic beta-cells in a subject in need thereof comprising: preparing iPSCs and/or pancreatic-beta-cells in vitro; treating said iPSCs or pancreatic beta-cells with an agent for gene editing of RPLP1, wherein the gene editing causes expression of an RPLP1 mutant comprising one or more phosphomimetic amino acids in the cell; and transplanting the treated iPSCs or pancreatic beta-cells into the subject, wherein the transplanting increases insulin levels in the subject and/or decreases glucose levels in the subject. In some cases, the subject has T1 or T2 diabetes, or alternatively is at risk for T2 diabetes. For example, in some cases the subject may have pre-diabetes such as an HbA1c level of 5.8-6.5% or 5.8-6.4%, or may have a family history of T1 or T2 diabetes.

Embodiment 20: The method of embodiment 18 or 19, wherein the RPLP1 mutant comprises one or more phosphomimetic amino acid mutations at an amino acid that is a serine in wildtype RPLP1.

Embodiment 21: The method of embodiment 20, wherein the serine in wildtype RPLP1 is serine 101 and/or serine 104.

Embodiment 22: The method of any one of embodiments 18-21, wherein the one or more phosphomimetic amino acid mutations is an aspartic acid or glutamic acid.

Embodiment 23. A composition comprising (1) an iPSC or pancreatic beta-cell and (2) a system for gene editing of RPLP1.

Embodiment 24. The composition of embodiment 23, wherein the system for gene editing is a CRISPR/Cas9 system, zinc-finger nuclease, TALEN, meganuclease, or GIIEE.

Embodiment 25. The composition of embodiment 23 or embodiment 24, wherein the system for gene editing is capable of introducing a phosphomimetic amino acid at positions serine 101 and/or serine 104.

Embodiment 26. The composition of embodiment 25, wherein the phosphomimetic amino acid mutation is an aspartic acid or glutamic acid.

Additional objects and advantages are set forth in part in the description which follows, and in part will be understood from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

FIGS. 1A-G show that sustained high glucose decreases basal insulin translation in isolated rat islets. Figure TA shows an embodiment of a general workflow showing hand-picked islets from adult Sprague-Dawley rats that were cultured for 4 days in media containing 5.5 mM (solid gray bars) or 16.7 mM (hatched gray bars) glucose (GLU). Following 1 hour rest in 2.8 mM GLU, GSIS quantified at 2.8 mM (basal, B) and 16.7 mM (stimulated, S) GLU. FIG. 1B shows the results of GSIS normalized by DNA with stimulation (stim) index quantified as stimulatory/basal secretion. FIG. 1C shows Insulin content normalized to DNA. FIG. 1D shows qPCR quantification of beta-cell markers Ucn3, Mafa, and Pdx1 relative to 18S rRNA. FIG. 1E shows cells pulse labeled with O-propargyl-puromycin (OPP) and analyzed by SDS-PAGE and showing total protein quantified by Coomassie stain (left) and newly synthesized protein quantified by click-addition of Alexa-647 (right). Representative images with quantification. FIG. 1F shows metabolic labeling with OPP, click-biotin addition and streptavidin pulldown of nascent proteins with immunoblot analysis of newly synthesized proinsulin (proINS) with tubulin control. FIG. 1G shows representative immunoblots and quantification for ER stress markers with thapsigargin (THAP)-treated control. Means±standard error (SE) for n=3-4 independent experiments. *′ P_{adj}<0.05 by 2-way ANOVA with Bonferroni post-hoc correction (1B [GSIS], 1D,1 G). ^{#}, P<0.05 by unpaired t-test (1B [stim index], 1C, 1E) or by ratio-paired t-test (1F). ns, not significant.

FIGS. 2A-2I show sustained high glucose decreases insulin synthesis in MIN6 cells. FIG. 2A shows an embodiment of a general workflow showing MIN6 cells incubated in media containing 5.5 mM (solid gray bars) or 25 mM (hatched gray bars) GLU for 24 hours. Following 1 hour rest in 2.8 mM GLU, GSIS was quantified at 2.8 mM (B) and 16.8 mM (S) GLU. FIG. 2B shows the results of GSIS normalized by cell number with stim index quantified as stimulatory/basal secretion. FIG. 2C shows Insulin content per 10⁶ cells.

FIG. 2D shows GSIS normalized to cellular insulin content. FIG. 2E shows GSIS normalized by cell number following incubation in 5.5 mM GLU, 25 mM GLU, or 5.5 mM GLU with 19.5 mM mannitol (MAN, open bars). FIG. 2F shows qPCR quantification of beta-cell markers Ucn3, Mafa, and Pdx1 relative to 18S rRNA. FIG. 2G shows OPP pulse labeling and SDS-PAGE with total protein quantified by Coomassie stain and newly synthesized protein quantified by click-addition of Alexa-647. Representative images with quantification. FIG. 2H shows OPP labeling, click-biotin addition, and streptavidin pulldown of nascent proteins with immunoblot for newly synthesized proINS and tubulin control. FIG. 2I shows Representative immunoblots and quantification for ER stress markers with tunicamycin (TN)-treated control. Means±SE for n=3-4 independent experiments. *, P_{adj}<0.05 by 2-way ANOVA with Bonferroni post-hoc correction (2B [GSIS], 2D, 2E, 2I). #, P<0.05 by unpaired t-test (2B [stim index], 2C, 2G) or by ratio-paired t-test (2H). ns, not significant.

FIGS. 3A-3I show sustained high glucose treatment has genome-wide impact on translation. FIG. 3A shows an embodiment of a workflow showing MIN6 cells incubated in media containing 5.5 mM or 25 mM GLU for 24 hours and analyzed by ribosome profiling using RNA sequence analysis of ribosome protected footprints (RPFs, translatome) and total RNA (transcriptome). FIG. 3B shows RPF read lengths with boxes indicating 25^{th} to 75^{th} percentiles, line in middle of box is median, and whiskers indicating smallest to largest values. FIG. 3C shows distribution of reads to coding sequence (CDS, hatched bars), 5′UTR (open bars), 3′UTR (solid bars) for RNAs and RPFs. FIG. 3D shows triplet periodicity of RPFs near CDS start and stop. FIGS. 3E, 3F, and 3G show volcano plots of −log FDR vs. log₂ fold change (FC), calculated for 25 mM vs. 5.5 mM GLU for RNA (3E, dotted line FDR=0.01), RPF (3F, dotted line FDR=0.1) and translation efficiency (TE=RPF/RNA, 3G, dotted line FDR=0.1). FIGS. 3H and 3I show representative Reactome gene sets over-represented at a significance threshold FDR<0.05 as upregulated (3H) and downregulated (3I) by 25 vs. 5.5 mM glucose. n=8 independent samples/condition.

FIGS. 4A-4G show sustained high glucose treatment has genome-wide impact on nascent proteome. FIG. 4A shows representative workflow in which MIN6 cells were incubated for 24 hours in media containing 5.5 mM or 25 mM GLU followed by nascent proteomics analysis. FIG. 4B shows surrogate variable PCA analysis. FIG. 4C shows volcano plot of −log FDR v. log₂ FC, calculated for 25 vs. 5.5 mM GLU. FIG. 4D shows correlation analysis of nascent proteomics Z-scores vs. RPF z-scores. FIGS. 4E and 4F show overlap of proteins upregulated (4E) or downregulated (4F) by 25 mM vs. 5 mM GLU in both TE and nascent proteomics datasets. −log FDR>1 and log₂ FC>20%. FIG. 4G shows representative RPF gene coverage plots for SCGN and IDH2, showing no evidence for new upstream open reading frames or pausing in 25 mM GLU. n=8 independent samples per condition. CDS, coding sequence.

FIGS. 5A-5B show translational regulation by sustained high glucose impacts protein abundance in MIN6 cells. MIN6 cells incubated for 24 h in media containing 5.5 mM (solid gray bars) or 25 mM (hatched gray bars) GLU. FIG. 5A shows qPCR quantification of polysome and total RNA for Ins1, Ins2, Scgn, Slc2a2, Pfkfb3, Slc30a8, Vps41, Idh2, and Igf2, relative to 18S rRNA. Actb and Tubg1 as controls. Means±SE for n=3-4 independent experiments. FIG. 5B shows immunoblot of cell lysates for SCGN, SLC2A2, PFKFB3, SLC30A8, VPS41, IDH2, and IGF2. Tubulin loading control. Tubulin for SLC2A2 panel and SLC30A8 panel are identical, since they were the same lanes on the gel. Representative blots with quantification of means±SE for n=3-7 independent experiments. *, P<0.05 by unpaired t-test (5A) or ratio-paired t-test (5B).

FIGS. 6A-6B show regulation by sustained high glucose impacts protein abundance in rat islets. Primary rat islets incubated for 4 days in media containing 5.5 mM (solid gray bars) or 16.7 mM (hatched gray bars) GLU. FIG. 6A shows qPCR quantification of ribosome-associated and total RNA for Ins1, Ins2, Scgn, Slc2a2, Pfkfb3, Slc30a8, Vps41, Idh2, and Igf2, relative to 18S rRNA. Actb and Tubg1 as controls. Means±SE for n=3-4 rats. FIG. 6B shows representative immunoblots of islet lysates for SCGN, SLC2A2, PFKFB3, SLC30A8, VPS41, IDH2, and IGF2. Tubulin loading control. Tubulin for SLC2A2 panel and SLC30A8 panel are identical, since they were same lanes on the gel. Quantification of means±SE for n=5-6 rats. *, P<0.05 by unpaired t-test (6A) or ratio-paired t-test (6B).

FIGS. 7A-7F show translational regulation by sustained high glucose impacts protein abundance in human islets. FIG. 7A shows work flow in which human cadaveric islets were cultured for 2 days in media containing 5.5 mM (solid gray bars) or 20 mM (hatched gray bars) GLU, followed by 1 hour rest in 2.8 mM GLU, and GSIS quantified at 2.8 mM (B) and 16.7 mM (S) GLU. FIG. 7B shows GSIS normalized by DNA with stim index quantified as stimulatory/basal secretion. FIG. 7C shows insulin content normalized to DNA. FIG. 7D shows GSIS normalized to cellular insulin content. FIG. 7E shows qPCR quantification of ribosome-associated and total RNA for INS, SCGN, SLC2A1, PFKFB3, SLC30A8, VPS41, IDH2, IGF2, and SLC2A2, relative to 18S rRNA. TUBG1 as control. FIG. 7F shows representative immunoblots of islet lysates for INS, SCGN, SLC2A1, PFKFB3, SLC30A8, and VPS41. Tubulin as control. Tubulin for SCGN panel and VPS41 panel are identical, since they were the same lanes on the gel. Quantification of means±SE for n=5 donors. *, Padj<0.05 by pre-planned paired t-test (Bonferroni post-hoc correction, 7B [GSIS], D). #, P<0.05 by unpaired t-test (7B [stim index], 7C), paired t-test (7E), or by ratio-paired t-test (7F). ns, not significant.

FIGS. 8A-8D show translational regulation by hyperglycemia impacts protein abundance in partial pancreatectomy model of hyperglycemia. FIG. 8A shows workflow in which islets were isolated from Sprague-Dawley rats 10 weeks after sham or 90% pancreatectomy (PX) surgery. FIG. 8B shows fed blood glucose. Means±SE for n=21 sham rats; n=29 PX rats. FIG. 8C shows qPCR quantification of ribosome-associated and total islet RNA for Ins1, Ins2, Scgn, Slc2a2, and Slc30a8, relative to 18S rRNA. Tubg1 as control for sham (solid gray bars) vs. PX (hatched gray bars). Means±SE for n=5-7 samples, each pooled from 2-3 rats. FIG. 8D shows representative immunoblots of islet lysates for INS, SCGN, SLC2A2, and SLC30A8 with tubulin as control. Tubulin for INS panel, SCGN panel, and SLC30A8 panel are identical, since they were the same lanes on the gel. Quantification of means±SE for n=5 samples, each pooled from 2-3 rats. *, P<0.05 by unpaired t-test (B, C, and D). ns, not significant.

FIGS. 9A and 9B show that sustained high glucose decreases RPLP1 on translating ribosomes. MIN6 cells treated with 25 mM vs. 5.5 mM glucose (GLU) for 24 hours. Actively translating ribosomes isolated by sucrose density gradient fractionation. FIG. 9A shows core ribosomal proteins quantified by tandem mass tag (TMT)-labeled proteomics. FIG. 9B shows immunoblot analysis of RPLP1 relative to other core ribosomal proteins in total cell lysates and actively translating polysomes with quantification. Means±SE for n=6 per condition. *, P<0.05.

FIGS. 10A and 10B show that sustained high glucose causes RPLP1 to dissociate from actively translating polysomes to a soluble pool. MIN6 cells were transduced with a lentivirus expressing wild type FLAG-tagged RPLP1. Following 24-hour treatment with 5.5 vs. 25 mM glucose (GLU), RPLP1 was assessed by immunoblotting of polysome (10A) and soluble fractions (10B). RPL27 and tubulin served as controls in polysome and soluble fractions, respectively. Means±SE for n=3 per condition. *, P<0.05.

FIG. 11 shows that mutation of serine 101 and serine 104 to the unphosphorylatable amino acid alanine mimics the effect of sustained high glucose on RPLP1 polysome association. MIN6 cells were transduced with lentiviruses expressing wild type FLAG-tagged RPLP1 or RPLP1^{S101A/S104A}. Anti-FLAG immunoblot was used to assess quantity of epitope-tagged wild type or mutant RPLP1 in lysate and in actively translating polysomes with RPL27 as a control. Means±SE for n=3 per condition. *, P<0.05

FIG. 12 shows a model of how RPLP1 localization is regulated by phosphorylation. RPLP1, an exchangeable acidic ribosomal protein, binds to the stalk of the large subunit. RPLP1 undergoes reversible phosphorylation of two serine residues (S101 and S104) near its carboxyl terminal. Soluble RPLP1 is unphosphorylated, whereas phosphorylation drives ribosome association. As described herein, sustained high glucose can cause RPLP1 to dissociate from ribosomes.

### DESCRIPTION OF THE SEQUENCES

A listing of certain sequences referenced herein is provided in Table 5 below. A sequence listing is also included in this application, as referenced above.

### DESCRIPTION OF CERTAIN EMBODIMENTS

### I. Ribosomal Remodeling in Diabetes

Ribosomes in eukaryotes are comprised of approximately 80 core ribosomal proteins and four ribosomal RNAs (rRNAs) (57). In some embodiments, diversity of ribosomes, and their function, is regulated by diversity and modifications of rRNA. In addition, ribosomal protein composition and modifications can also impact ribosomal diversity and function.

As described herein, “ribosomal remodeling” refers to a change in protein composition within a ribosome, such as a change in stoichiometry of various ribosomal proteins. In other words, the term ribosomal remodeling can be used to describe changes in ribosomal protein abundances within ribosomes. In some embodiments, ribosomal remodeling allows for cell-specific differences in ribosomal composition. In some embodiments, ribosomal remodeling is a mechanism for dynamic control of translation of mRNAs in response to changing environmental stimuli such as nutrients. As such, ribosomal remodeling can have regulatory effects on translation, leading to differences in expression of proteins within a cell.

In some embodiments, pharmacological or genetic interventions to drive proteins onto or off the ribosome for ribosomal remodeling can be used to effect changes in protein expression and cellular functions. In some embodiments, ribosomal proteins may dynamically associate and dissociate from ribosomes during ribosomal remodeling. In some embodiments, post-translational modifications of ribosomal proteins leads to ribosomal remodeling.

### A. RPLP1 Composition in Ribosomes

Ribosome-associated ribosomal protein P1 (also known as large ribosomal subunit protein P1 or RPLP1, UniProt entry P05386) is an acidic protein that binds to the flexible P-stalk of the large ribosomal subunit, which forms part of the GTPase-associated center where translation factors engage and where GCN2 interacts to regulate translation in response to nutrient stress (58, 59). Unlike most core ribosomal proteins, RPLP1 exchanges between ribosome-bound and soluble protein pools (60). When bound to the ribosome, RPLP1 is phosphorylated on two serine residues (S101 and S104) near its carboxyl terminus, whereas soluble RPLP1 is dephosphorylated (61-64). Moreover, phosphorylation is required for and drives ribosome association of RPLP1 (65-67).

The amino acid sequence of RPLP1 is shown here, with underlining of serine 101 and 104:

(SEQ ID NO: 1)
MASVSELACIYSALILHDDEVTVTEDKINALIKAAGVNVEPFWP
GLFAKALANVNIGSLICNVGAGGPAPAAGAAPAGGPAPSTAAAP
AEEKKVEAKKEESEESDDDMGFGLFD.

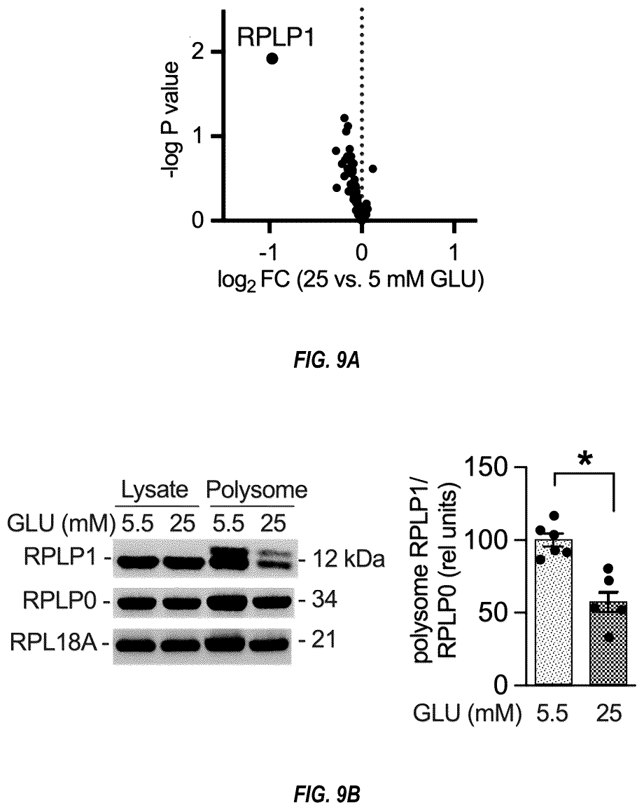

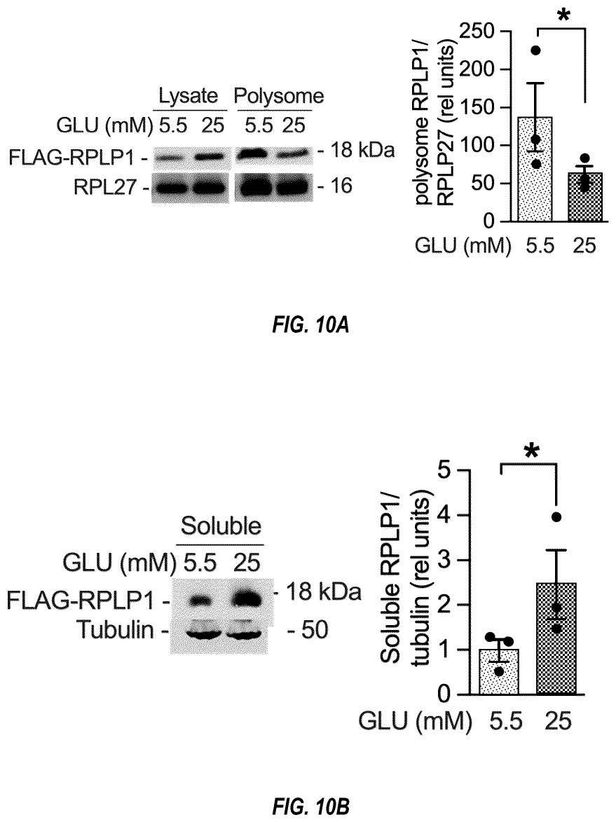

The present disclosure shows changes in RPLP1 associated with ribosomes in high glucose conditions (see, for example, FIGS. 9A and 9B). In some embodiments, hyperglycemic conditions may increase the percentage of soluble RPLP1 and/or decrease the amount of ribosome-associated RPLP1 (FIG. 10). Described herein are means of blocking ribosomal remodeling based on RPLP1, such as with kinase activators or phosphatase inhibitors. Also described are means of blocking this ribosomal remodeling with gene editing of RPLP1.

In some embodiments, ribosomal remodeling occurs in pancreatic beta-cells. In some embodiments, the presence of RPLP1 within ribosomes in pancreatic beta-cells is altered due to the process of ribosomal remodeling. In some embodiments, association of RPLP1 with the ribosome increases insulin translation and secretion in pancreatic beta-cells. In some embodiments, phosphorylation of RPLP1 leads the protein to associate with the ribosome and dephosphorylation leads the protein to dissociation from the ribosome. In some embodiments, association of RPLP1 with the ribosome can be measured using isolation of actively translating ribosomes by sucrose density gradient fractionation (as shown in FIG. 9A). In some embodiments, targeting posttranslational modification of RPLP1 affects its incorporation into ribosomes, which could have therapeutic implications with respect to insulin secretion.

### II. Methods of Treating or Preventing Diabetes

In some embodiments, diabetes in a patient is treated or prevented by regulating RPLP1. In some embodiments, the subject has Type I (T1) or Type II (T2) diabetes. In some embodiments, the subject is at risk for T2 diabetes.

The term “treating” or “treatment,” as used herein, covers any administration or application of a therapeutic for disease in a subject, and includes inhibiting the disease, arresting its development, relieving one or more symptoms of the disease, or preventing reoccurrence of one or more symptoms of the disease. For example, treatment of type 1 diabetes subjects may comprise alleviating hyperglycemia as compared to a time point prior to administration or reducing the subject's need for exogenous insulin administration.

The term “preventing” or “prevention,” as used herein, covers any administration or application of a therapeutic for disease in a subject for the purpose of blocking the occurrence of one or more symptoms of the disease. For example, preventing diabetes may include administration or application of a therapeutic in a subject with a family history of diabetes type 1, wherein the incidence of hyperglycemia or immune attack on pancreatic beta-cells associated with type 1 diabetes is reduced by the administration.

In each embodiment of the invention, the subject treated is a mammal. In one embodiment, the mammal is a human, non-human primate, cow, horse, pig, sheep, goat, dog, cat, or rodent. In embodiment, the subject is a human subject.

Glucose levels in the blood are normally tightly regulated to maintain an appropriate source of energy for cells of the body. Dysregulation of blood sugar must be ameliorated to maintain health and longevity, and therapies that are fast acting are especially desired. Such fast acting therapies allow subjects to monitor blood glucose in real time and immediately self-medicate themselves to bring glucose levels within normal limits. Dosing with exogenous insulin is one example of a fast-acting glucose modulator that has allowed subjects with diabetes to maintain relatively normal lifestyles. Described herein is a non-insulin fast-acting compound that regulates blood glucose levels in real-time.

Insulin and glucagon are principal hormones that regulate blood glucose levels. In response to an increase in blood glucose, such as after a meal, insulin is released from beta-cells of the pancreas. Insulin regulates the metabolism of carbohydrates and fats by promoting uptake of glucose from the blood into fat and skeletal muscle. Insulin also promotes fat storage and inhibits the release of glucose by the liver. Regulation of insulin levels is a primary means for the body to regulate glucose in the blood.

When glucose levels in the blood are decreased, insulin is no longer released and instead glucagon is released from the alpha cells of the pancreas. Glucagon causes the liver to convert stored glycogen into glucose and to release this glucose into the bloodstream. Thus, insulin and glucagon work in concert to regulate blood glucose levels.

In one embodiment, treatment of diabetes mellitus is to administer a composition to a subject to lower blood glucose.

Hyperglycemia refers to an increased level of glucose in the blood as compared to the levels normally seen in a healthy subject. Hyperglycemia can be associated with high levels of sugar in the urine, frequent urination, and increased thirst. Diabetes mellitus refers to a medical state of hyperglycemia.

The American Diabetes Association (ADA) suggests that fasting plasma glucose (FPG) levels of 100 mg/dL to 125 mg/dL or HbA1c levels of 5.7% to 6.4% may be considered hyperglycemia and may indicate that a subject is at high risk of developing diabetes mellitus (i.e. prediabetes, see ADA Guidelines 2015).

The ADA states that a diagnosis of diabetes mellitus may be made in a number of ways. A diagnosis of diabetes mellitus can be made in a subject displaying an HbA1c level of ≥6.5%, an FPG levels of ≥126 mg/dL, a 2-hour plasma glucose of ≥200 mg/dL during an OGTT, or a random plasma glucose level≥200 mg/dL in a subject with classic symptoms of hyperglycemia.

Diabetes mellitus can be classified as Type 1 or Type 2. Type 1 diabetes mellitus (previously known as insulin-dependent diabetes or juvenile diabetes) is an autoimmune disease characterized by destruction of the insulin-producing beta-cells of the pancreas. Classic symptoms of Type 1 diabetes mellitus are frequent urination, increased thirst, increased hunger, and weight loss. Subjects with Type 1 diabetes mellitus are dependent on administration of insulin for survival.

Type 2 diabetes mellitus is a metabolic disease characterized by a relative decrease in insulin levels and/or a phenotype of insulin resistance. Insulin resistance refers to when cells of the body no longer respond appropriately to insulin. The risk of Type 2 diabetes mellitus is increased in individuals who are obese or who have a sedentary lifestyle.

In the absence of regulation of glucose levels in subjects with diabetes, a range of serious complications may be seen. These include atherosclerosis, kidney disease, stroke, nerve damage, and blindness.

A method of treating diabetes mellitus comprising administering a composition is encompassed. In one embodiment, the method comprises lowering blood glucose levels in the diabetic subject to below about 200 mg/dL, 150 mg/dL, 100 mg/dL, or about 125 mg/dL.

In some embodiments, treatment of diabetes is increasing insulin levels in the subject after administering a composition.

In some embodiments, administering a composition causes a decrease in blood glucose levels such that levels are less than 200 mg/dL.

In some embodiments, the subject treated with a composition has Type 1 diabetes mellitus. In some embodiments, the diabetic subject treated has a relative decrease in insulin levels as compared to a healthy control subject. In some embodiments, the subject treated has decreased beta-cell mass as compared to a healthy control subject. In some embodiments, the decrease in beta-cell mass in a subject is due to an autoimmune disease.

In some embodiments, the subject treated has diabetes mellitus based on diagnosis criteria of the American Diabetes Association. In some embodiments, the subject with diabetes mellitus has an HbA1c level of ≥6.5%. In some embodiments, the subject with diabetes mellitus has fasting plasma glucose (FPG) levels of ≥126 mg/dL. In some embodiments, the subject with diabetes mellitus has a 2-hour plasma glucose of ≥200 mg/dL during an oral glucose tolerance test (OGTT). In some embodiments, the subject with diabetes mellitus has a random plasma glucose level≥200 mg/dL or 11.1 mmol/L. In some embodiments, the subject with diabetes mellitus has a random plasma glucose level≥200 mg/dL or 11.1 mmol/L with classic symptoms of hyperglycemia. In some embodiments, administering an agent as described herein improves one or more of these markers of diabetes.

In some embodiments, treating diabetes in a subject reduces the subject's HbA1c level to 6.5% or less. In some embodiments, treating diabetes in a subject reduces the subject's FPG levels to 126 mg/dL or less. In some embodiments, treating diabetes in a subject reduces the subject's 2-hour plasma glucose during an OGTT to 200 mg/dL or less. In some embodiments, after treatment, a subject with diabetes mellitus has a random plasma glucose level of 200 mg/dL or less or 11.1 mmol/L or less without classic symptoms of hyperglycemia after treating diabetes in the subject.

In some embodiments, a method herein is performed to prevent diabetes and/or to reduce the incidence of hyperglycemia or immune attack on pancreatic beta-cells associated with type 1 diabetes in the subject. In some embodiments, the subject's HbA1c level is 6.5% or less when diabetes is prevented. For example, in some embodiments, the subject has an HbA1c level of 5.8-6.5% or of 5.8-6.4%. In some embodiments, the subject's FPG levels are 126 mg/dL or less when diabetes is prevented. In some embodiments, the subject's 2-hour plasma glucose during an OGTT is 200 mg/dL or less when diabetes is prevented. In some embodiments, a subject with diabetes mellitus has a random plasma glucose level of 200 mg/dL or less or 11.1 mmol/L or less without classic symptoms of hyperglycemia when diabetes is prevented. For example, in some cases, incidence of hyperglycemia or immune attack on pancreatic beta-cells associated with type 1 diabetes is reduced in the subject, and/or the subject's HbA1c level remains 6.5% or less following administration.

### A. Changes in RPLP1 in High Glucose Conditions

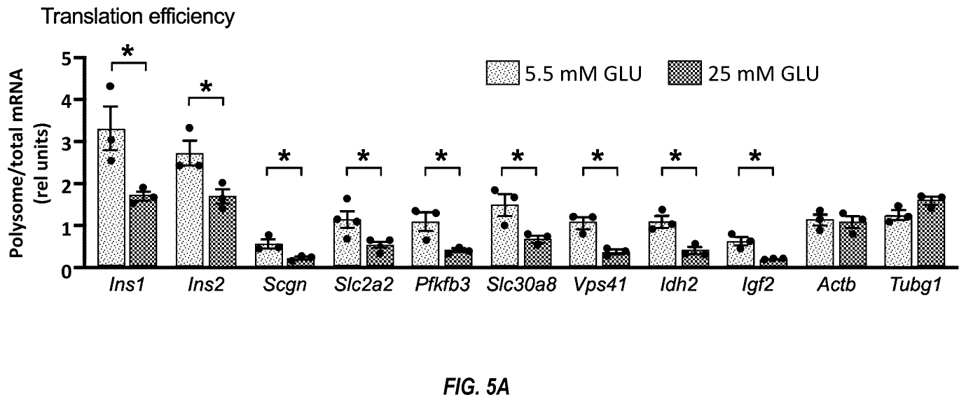

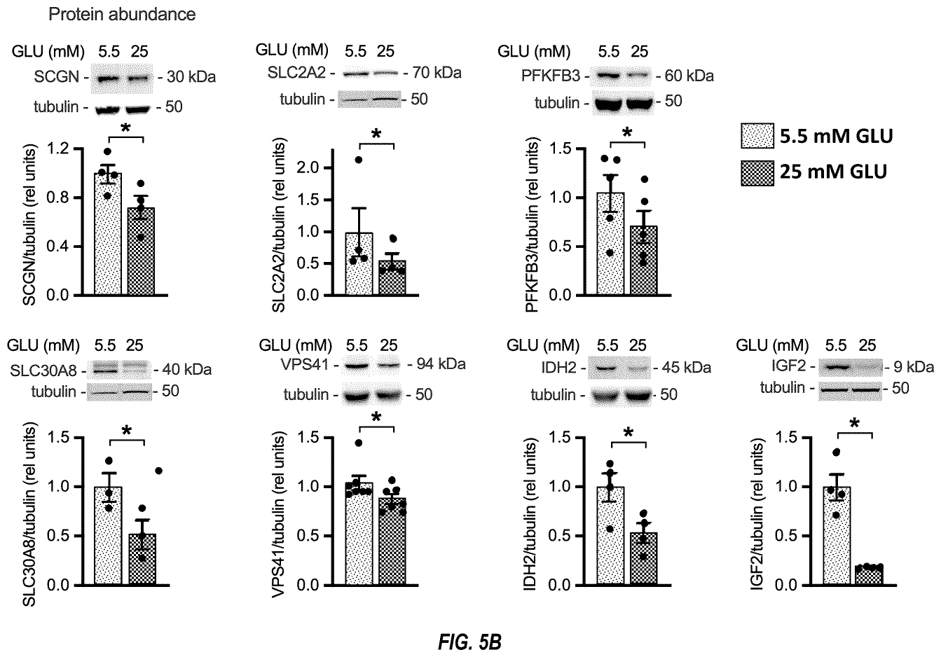

As described herein, translation of a number of proteins critical for insulin secretion by pancreatic beta-cells is decreased by high glucose conditions (see, for example, Table 2 below) and leads to decreased steady-state abundance of these proteins (see, for example, FIGS. 5, 6, and 7). Such a decrease in expression of insulin synthesis/secretion genes could initiate conditions wherein initial high glucose levels are exacerbated by deficiencies in insulin synthesis or secretion. As described herein, decreases in the percentage of RPLP1 in ribosomes and/or increases in percentage of soluble RPLP1 could lead to these detrimental changes in protein translation.

In some embodiments, a method of treating or preventing diabetes in a subject in need thereof comprises increasing the percentage of ribosome-associated RPLP1 or decreasing the percentage of soluble RPLP1 in a cell of the subject. In some embodiments, this method comprises phosphorylating RPLP1, inhibiting dephosphorylation of RPLP1, and/or expressing an RPLP1 mutant comprising one or more phosphomimetic amino acids in the cell, thereby increasing the percentage of ribosome-associated RPLP1 and/or decreasing the percentage of soluble RPLP1 in the cell and/or in the subject. In some embodiments, increasing the percentage of ribosome-associated RPLP1 or decreasing the percentage of soluble RPLP1 in the subject is in comparison to the same subject before the treating. In some embodiments, increasing the percentage of ribosome-associated RPLP1 or decreasing the percentage of soluble RPLP1 in the cell is in comparison to the cell before the treating. In some embodiments, high glucose cellular conditions in a subject with diabetes leads to a low percentage of RPLP1 associated with the ribosome and/or a high level of soluble RPLP1 in the subject, and a treatment described herein can reverse these effects of high glucose. In some embodiments, association of RPLP1 with the ribosome can be measured using isolation of actively translating ribosomes by sucrose density gradient fractionation.

In some embodiments, increased RPLP1 phosphorylation at serine 101 and/or serine 104 of SEQ ID NO: 1 increases incorporation of RPLP1 into ribosomes as compared to RPLP1 that is not phosphorylated at serine 101 and/or serine 104. In some embodiments, increased RPLP1 phosphorylation at serine 101 and/or serine 104 of SEQ ID NO: 1 decreases levels of soluble RPLP1 in cells as compared to the same cells before the treatment. In some embodiments, increased RPLP1 phosphorylation at serine 101 and/or serine 104 of SEQ ID NO: 1 in a pancreatic beta-cell changes expression of one or more proteins in the beta-cell as compared to the expression before treatment. In some embodiments, the change in expression is in one or more proteins associated with insulin production and/or secretion. In any of these embodiments, inhibiting dephosphorylation of RPLP1 may have the same effect as increasing phosphorylation of RPLP1.

In some embodiments, increasing the percentage of ribosomes containing RPLP1 increases translation of one or more proteins encoded by a gene selected from INS (gene encoding insulin; Gene ID: 3630), SCGN (gene encoding secretagogin; GENE ID: 10590), IDH2 (gene encoding isocitrate dehydrogenase; GENE ID: 3418), VPS41 (gene encoding VPS41 subunit of HOPS complex: GENE ID: 27072), SLC2A1 (solute carrier family 2 member 1; GENE ID: 6513), IGF2 (insulin like growth factor; GENE ID: 3481), SLC30A8 (solute carrier family 30 member 8; GENE ID: 169026), and PFKFB3 (6-phosphofructo-2-kinase/fructose-2,6-bisphosphatase 3; GENE ID: 5209). In some embodiments, the increased translation of the one or more proteins is in comparison to the same cell before being treated with an agent as described herein.

In some embodiments, administering a kinase activator or phosphatase inhibitor increases levels of phosphorylated RPLP1 in a cell, as compared to levels of phosphorylated RPLP1 before the administering. In some embodiments, administering a kinase activator or phosphatase inhibitor decreases levels of dephosphorylated RPLP1 in a cell, as compared to levels of dephosphorylated RPLP1 before the administering. In some embodiments, administering a system for gene editing causes expression of an RPLP1 mutant comprising one or more phosphomimetic amino acids. In some embodiments, the administering of the kinase activator or phosphatase inhibitor or of the system for gene editing can be by a buccal, enteral, inhalable, infused, intramuscular, intrathecal, intravenous, nasal, ophthalmic, oral, otic, rectal, subcutaneous, sublingual, topical, or transdermal route. In some embodiments, the administering of a gene editing system, for example, may be to a cell in vitro.

In some embodiments, the cell of the subject is a pancreatic beta-cell. In some embodiments, the method increases the levels of insulin within or released by the cell as compared to levels before administering of an agent described herein. As such, the method can improve symptoms or diabetes and/or decrease blood sugar levels as compared to before the administering.

### B. Methods of Regulating RPLP1 Composition in Ribosomes by Kinases and Phosphatases

Kinases and phosphatases are druggable targets, and pharmacologically activating the kinase responsible for RPLP1 phosphorylation or inhibiting the phosphatase that dephosphorylates the protein would be an approach to increase RPLP1 association with the ribosome.

In some embodiments, RPLP1 phosphorylation is increased by treatment of a cell with a kinase activator. In some embodiments, RPLP1 phosphorylation is increased by treatment of a cell with a phosphatase inhibitor. In some embodiments, the increase in RPLP1 phosphorylation is in comparison to the same cell before treatment. In any of these embodiments, inhibiting dephosphorylation of RPLP1 may have the same effect as increasing phosphorylation of RPLP1.

In some embodiments, RPLP1 phosphorylation is measured using radiolabeling or Western blot or immunoprecipitation with a phosphospecific antibody or mass spectrometry.

In some embodiments, the RPLP1 phosphorylation is at serine 101 of SEQ ID NO: 1. In some embodiments, the RPLP1 phosphorylation is at serine 104 of SEQ ID NO: 1. In some embodiments, the RPLP1 phosphorylation is at both serine 101 and serine 104 of SEQ ID NO: 1.

In some embodiments, increasing RPLP1 phosphorylation at serine 101 and/or 104 by treatment of a cell with a kinase activator or phosphatase inhibitor increases the percentage of RPLP1 associated with the ribosome as compared to the same cell before treatment. In some embodiments, increasing RPLP1 phosphorylation at serine 101 and/or 104 by treatment of a cell with a kinase activator or phosphatase inhibitor increases the percentage of RPLP1 associated with the ribosome as compared to the same cell before treatment.

In some embodiments, increasing RPLP1 phosphorylation or inhibiting dephosphorylation of RPLP1 at serine 101 and/or 104 by treatment with a kinase activator or phosphatase inhibitor leads to a change in expression of one or more protein in the cell. In some embodiments, the one or more protein is associated with insulin synthesis and/or secretion. In some embodiments, increasing RPLP1 phosphorylation or inhibiting dephosphorylation of RPLP1 at serine 101 and/or 104 by treatment with a kinase activator or phosphatase inhibitor increases translation of one or more proteins encoded by a gene selected from INS, SCGN, IDH2, VPS41, SLC2A1, IGF2, SLC30A8, and PFKFB3. In some embodiments, the increased translation of one or more proteins is in relation to the level of translation of the same protein in the same cell before treatment with an agent described herein.

In some embodiments, the cell treated is a pancreatic beta-cell, and the subject is treated with a kinase activator or phosphatase inhibitor.

In some embodiments, insulin levels in the subject are increased and/or glucose levels in the subject are decreased by the treatment in comparison to the levels in the patient before treatment. As such, the subject may be able to reduce or eliminate treatment with other agents for diabetes management, such as insulin or other pharmacologic therapy.

### C. Methods of Regulating RPLP1 Composition in Ribosomes by Gene Editing

In some embodiments, an RPLP1 mutant is prepared comprising one or more phosphomimetic amino acids. As used herein, a “phosphomimetic amino acid” refers to an amino acid substitution in a protein that mimics a phosphorylated protein. In some embodiments, a phosphomimetic amino acid has the same characteristics as the phosphorylated wildtype protein (without the substitution). A RPLP1 mutant comprising a phosphomimetic amino acid may have the phosphomimetic amino acid at a position that can be phosphorylated physiologically, such as a serine, tyrosine, or threonine.

In some embodiments, the phosphomimetic amino acid is at position S101 and/or position S104 of SEQ ID NO: 1, and the characteristics of RPLP1 comprising the phosphomimetic amino acid is the same or similar to the characteristics of wildtype RPLP1 that is phosphorylated at that position. In some embodiments, the one or more phosphomimetic amino acid mutation is an aspartic acid or glutamic acid. In some embodiments the RPLP1 mutant comprises a S101D or S104D substitution in SEQ ID NO: 1. In some embodiments the RPLP1 mutant comprises a S101E or S104E substitution in SEQ ID NO: 1. In some embodiments, the RPLP1 mutant comprises a S101D or S140D substitution together with a S101E or S104E substitution.

In some embodiments, expression of a RPLP1 mutant comprising one or more phosphomimetic amino acids is performed by gene editing of RPLP1. In some embodiments, the gene editing is performed with a system for gene editing.

A wide range of gene editing systems are well-known in the art (68 and 69). In some embodiments, a system for gene editing comprises a CRISPR/Cas9 system, zinc-finger nuclease, transcription activator-like effector nuclease (TALEN), meganuclease, or group one intron encoded endonuclease (GIIEE), However, the present methods are not limited to these specific gene editing systems, as one skilled in the art would be well-aware that new systems from gene editing are being rapidly developed.

In some embodiments, the cell treated is an iPSC or pancreatic beta-cell in culture and gene editing is performed in vitro. In some embodiments, the treated cell is introduced into the subject after the gene editing. In some embodiments, the iPSC or pancreatic beta-cell is taken from the patient, treated with a system for gene editing in vitro, and reintroduced to the patient. In some embodiments, an iPSC or pancreatic beta-cell from a donor is treated with a system for gene editing in vitro and introduced to the patient. In other words, the iPSC or pancreatic beta-cell may be used for a homologous transplant (i.e., the donor of the cell and the patient treated are the same) or the iPSC or pancreatic beta-cell may be used for a heterologous transplant (i.e., the donor of the cell and the patient treated are the different).

In some embodiments, the cell is an iPSC or pancreatic beta-cell in a subject and gene editing is performed in vivo using a delivery system that selectively delivers the gene editing system to the iPSC or pancreatic beta-cell. A number of different means to target a delivery system to iPSC or pancreatic beta-cells are known in the art (68). For example, a virus that targets iPSCs or pancreatic beta-cells may be used to deliver a gene editing system. In this way, the gene editing system may be targeted in vivo to the iPSC or pancreatic beta-cell.

In some embodiments, a method of treating or preventing diabetes in a subject in need thereof comprises preparing iPSCs and/or pancreatic beta-cells in vitro; treating said iPSCs or pancreatic beta-cells with an agent for gene editing of RPLP1, wherein the gene editing causes expression of an RPLP1 mutant comprising one or more phosphomimetic amino acids in the cell; and transplanting the treated iPSCs or pancreatic beta-cells into the subject, wherein the transplanting increases insulin levels in the subject and/or decreases glucose levels in the subject in comparison to the levels in the subject before the transplanting.

In some embodiments, an RPLP1 mutant comprises one or more phosphomimetic amino acid mutations at an amino acid that is a serine in wildtype RPLP1. In some embodiments, the serine in wildtype RPLP1 is serine 101 and/or serine 104. In some embodiments, the one or more phosphomimetic amino acid mutation is an aspartic acid or glutamic acid.

### III. Compositions Comprising a System for Gene Editing

Also described herein are compositions comprising (1) an iPSC or pancreatic beta-cell and (2) a system for gene editing of RPLP1. In some embodiments, the system for gene editing is a CRISPR/Cas9 system, zinc-finger nuclease, TALEN, meganuclease, or GIIEE, although any gene editing system may be used. In some embodiments, the system for gene editing is capable of introducing a phosphomimetic amino acid at positions serine 101 and/or serine 104. In some embodiments, the phosphomimetic amino acid mutation is an aspartic acid or glutamic acid.

### EXAMPLES

### Example 1. Sustained High Glucose Impairs Basal Insulin Translation

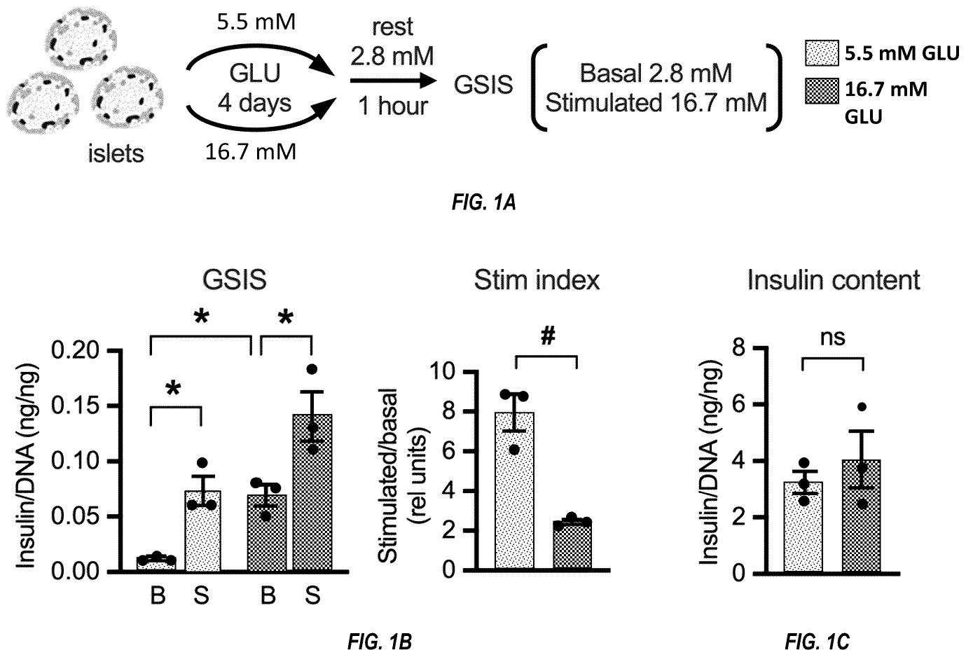

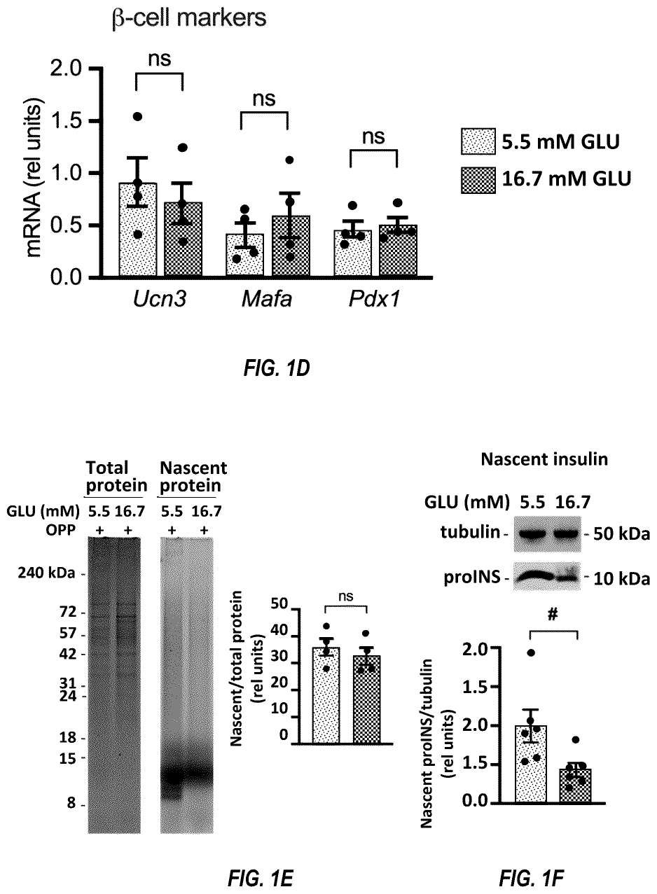

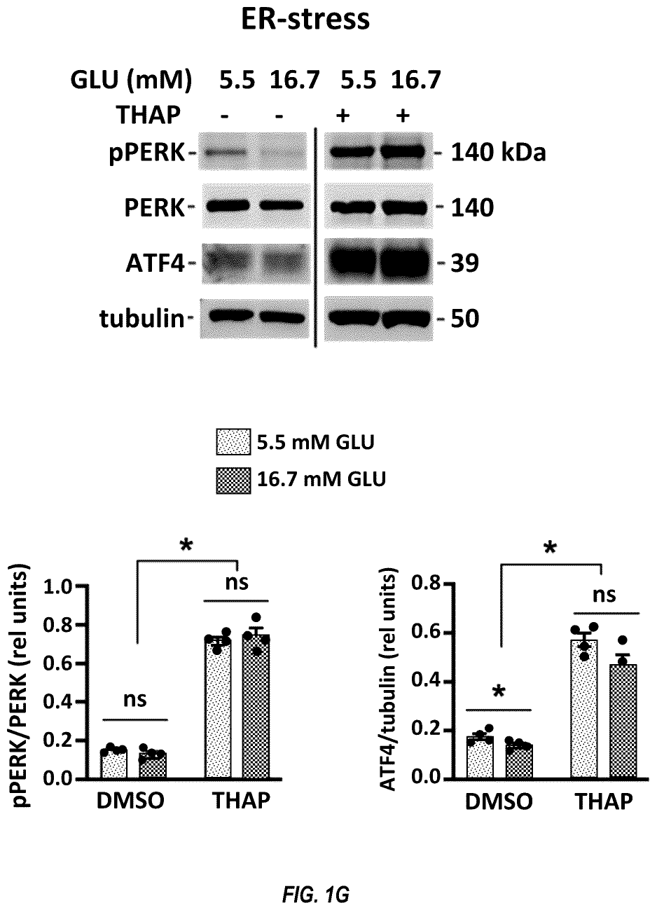

Chronic high glucose impairs glucose-stimulated insulin translation and secretion in human and rodent islets (14, 15). To determine whether sustained high glucose affects basal insulin translation, chronic high glucose exposure was modeled by incubating isolated rat islets in media containing 16.7 mM glucose versus 5.5 mM glucose. Islets were then rested in media with 2.8 mM glucose for 1 hour prior to assaying for GSIS (FIG. 1A). Compared to low glucose, islets incubated in high glucose for 4 days had increased basal insulin secretion and diminished response to stimulatory glucose with a 69% decrease in stimulation index, without impact on total islet insulin content (FIGS. 1B-1C). Shorter incubations in high glucose did not decrease GSIS (not shown). Exposure to high glucose over 4 days did not cause dedifferentiation or transdifferentiation, as expression of beta-cell identity genes was similar between the two glucose conditions (FIG. 1D). Although global protein synthesis was unchanged between the glucose conditions, sustained high glucose decreased the rate of insulin synthesis (FIG. 1E-1F).

Pancreatic beta-cells are particularly susceptible to increased ER stress and prolonged ER stress is detrimental to beta-cell function (19, 20). However, treatment with high glucose over 4 days did not increase the phosphorylation of PERK or expression of ATF4, well-established upstream regulators of ER stress (FIG. 1G).

Thus, 4 days exposure to sustained high glucose decreased basal translation of insulin prior to suppression of global protein synthesis, compromised beta-cell identity, or sustained engagement of the unfolded protein response pathway.

### Example 2. MIN6 Cells Model Chronic High Glucose Effects on Islet Insulin Translation

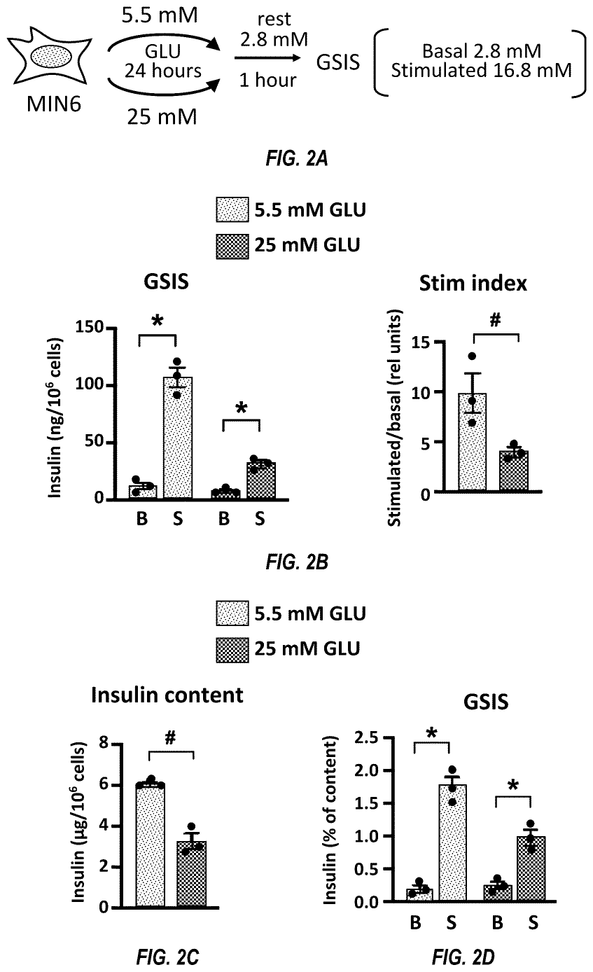

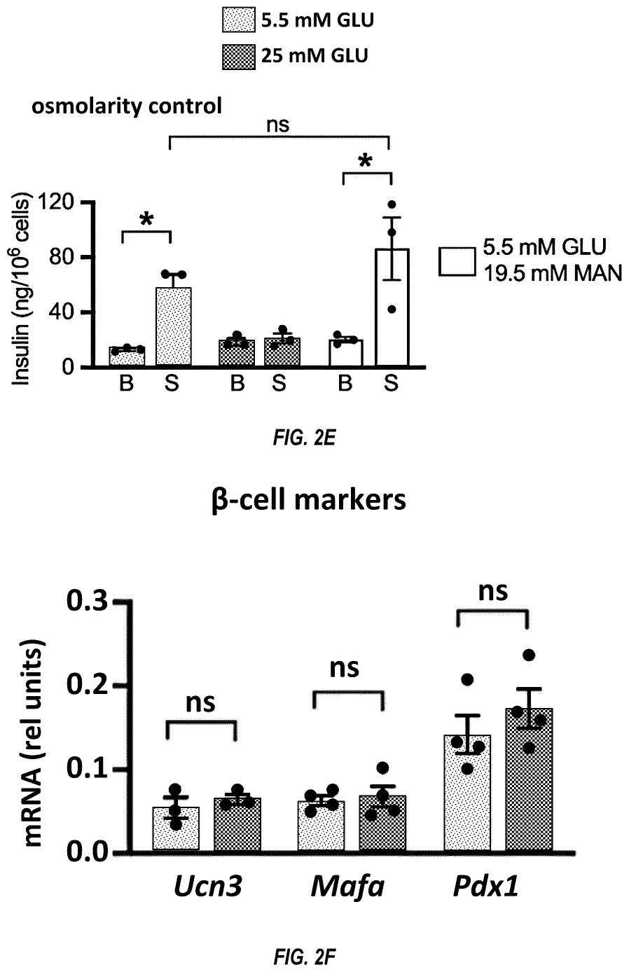

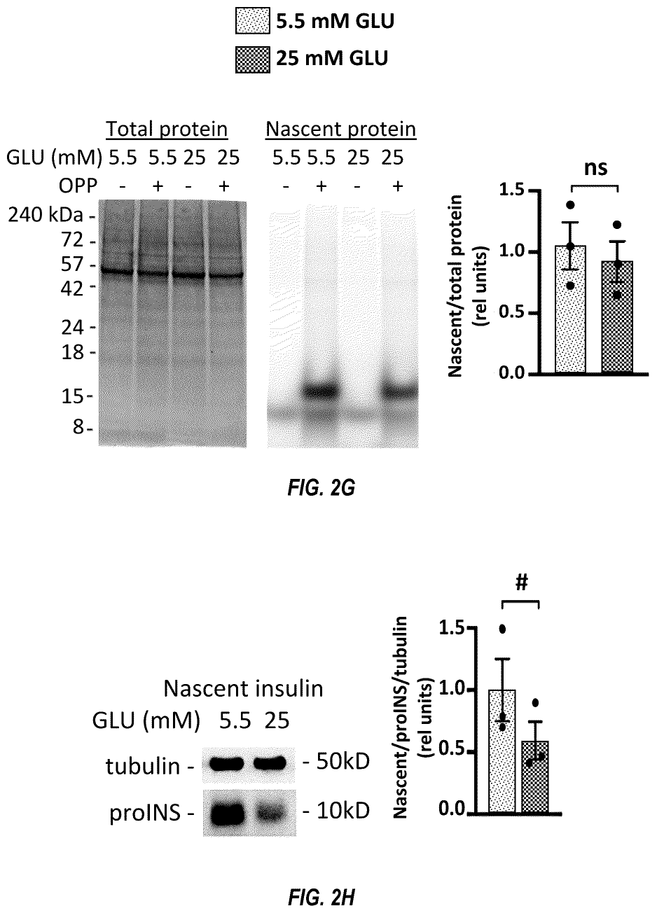

Pancreatic islets are micro-organs that consist of several cell types including glucagon-containing α-cells, somatostatin-containing δ-cells, and polypeptide-producing PP-cells in addition to insulin-producing beta-cells. To delineate the effects of chronic high glucose specifically on beta-cells and to identify a system that more readily provides sufficient material for high-throughput analyses, chronic high glucose exposure was modeled in early passage MIN6 insulinoma cells that support robust GSIS (21). These cells are typically propagated in 25 mM glucose to sustain rapid cell growth but can be maintained for limited periods in lower glucose with slower growth. Following incubation for 24 hours in 5.5 mM glucose, stimulatory glucose caused a 10-fold increase in insulin secretion (FIG. 2A-2B). Basal insulin secretion did not increase in MIN6 cells maintained in high glucose, contrary to observations in islets, but similar to islets, the stimulation index was decreased by 59% by high glucose. Although high glucose decreased insulin content of MIN6 cells, the impact of sustained high glucose on GSIS remained significant even when secretion was calculated as a percent of insulin content (FIG. 2C-2D). This did not reflect a general response to increased osmolarity, since incubation in low glucose media supplemented with mannitol did not recapitulate the effect of sustained high glucose (FIG. 2E). As observed in rat islets, beta-cell identity markers were similar in both glucose conditions, and sustained high glucose specifically decreased insulin translation without affecting global protein synthesis or inducing ER stress (FIG. 2F-21). Taken together, these results demonstrate that 24-hour treatment of MIN6 cells with 25 mM glucose vs 5.5 mM glucose largely models the effects of sustained high glucose treatment of primary rodent islets.

### Example 3. Broad Impact of Sustained High Glucose on Gene-Specific mRNA Translation Based on Ribosomal Profiling

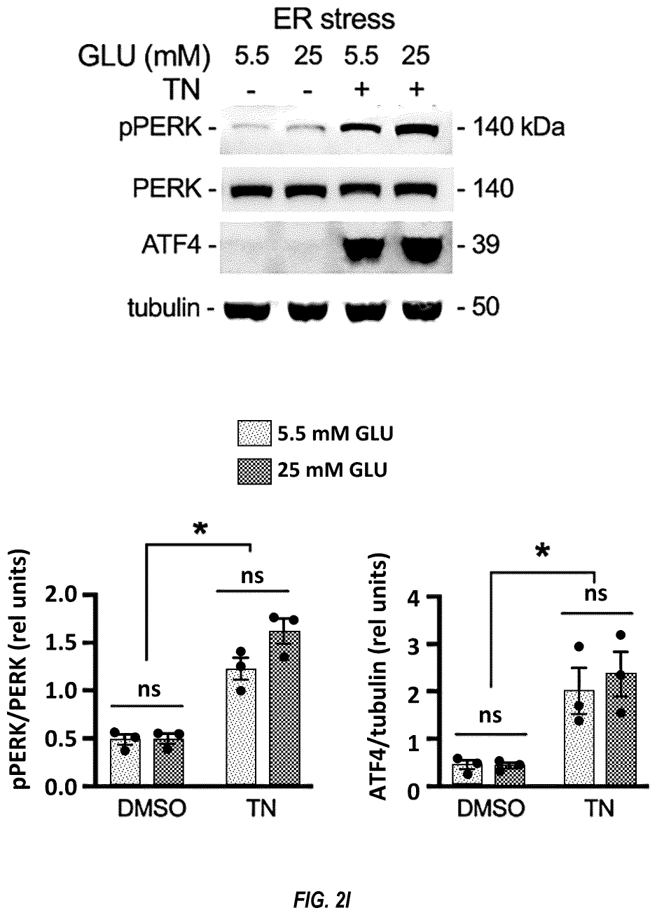

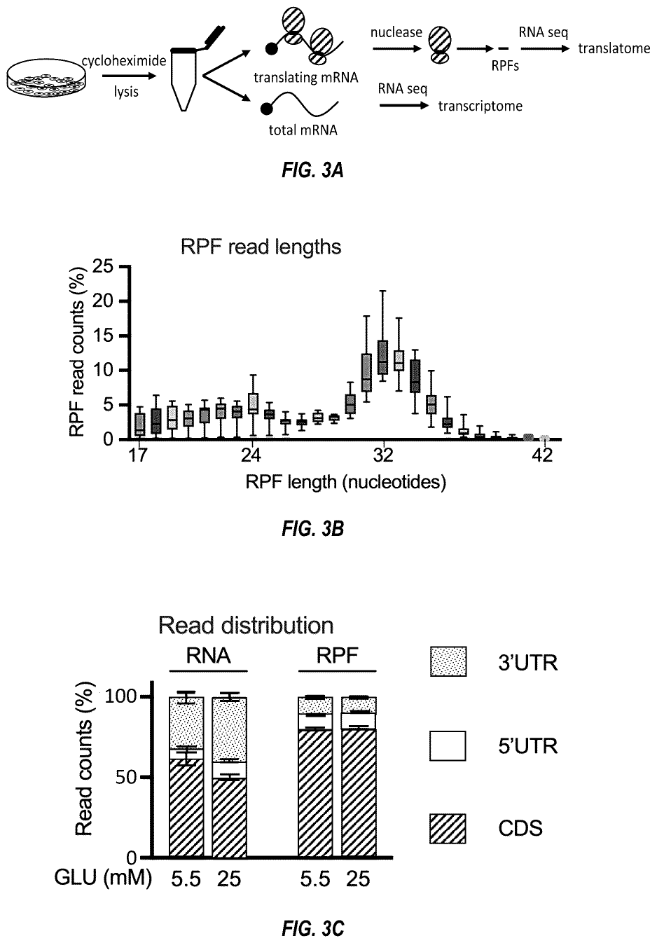

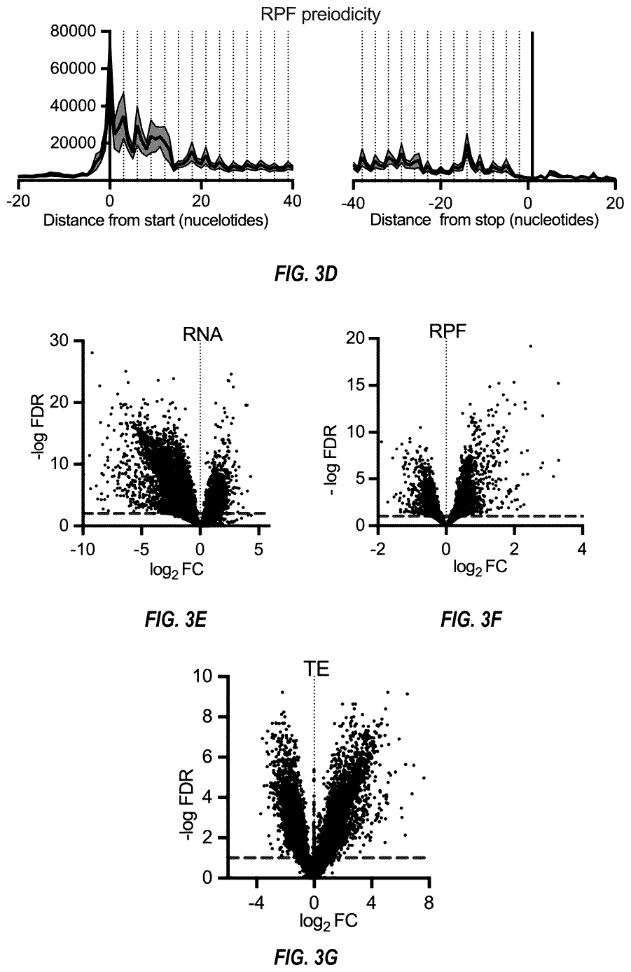

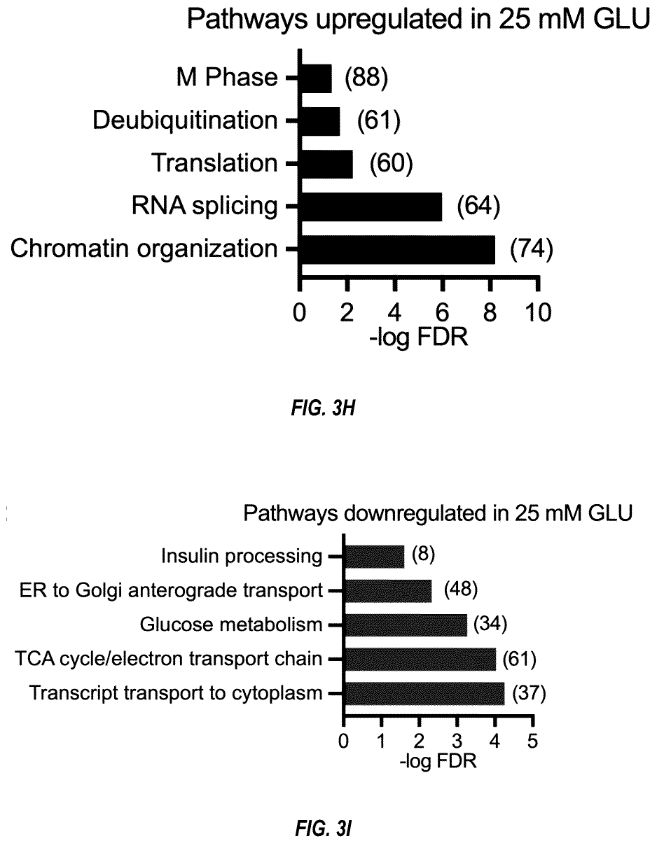

Incubation of MIN6 cells or islets in high glucose media supplemented with high concentrations of the saturated fatty acid palmitate induces ER stress and has profound effects on mRNA translation (17, 18). To determine the genome-wide effects of sustained high glucose alone on beta-cell mRNA translation in the absence of ER stress, the translatome of MIN6 cells in low vs. high glucose was evaluated by ribosome profiling. This RNA-sequencing method is based on the principle that more efficiently translated mRNAs are associated with more ribosomes and therefore generate more ribosome protected footprints (RPFs) upon nuclease digestion. Both RPF and total RNA libraries were sequenced from cells following treatment for 24 hours with 25 mM vs. 5.5 mM glucose (FIG. 3A). As observed in other ribosome profiling studies of mammalian cells (22), peak RPF fragment sizes were 30-35 base pairs, RPFs were enriched for open reading frames of genes compared to mRNAs, and RPF sequences showed triplet periodicity (FIG. 3B-3D). Sustained high glucose had substantial impact on the transcriptome (FIG. 3E), consistent with prior studies (16), and on the translatome (FIG. 3F). To identify genes for which glucose treatment specifically altered mRNA translation regulation, the translation efficiency (TE) was calculated as the ratio of normalized RPF reads to normalized total mRNA reads per gene (FIG. 3G). This measure accounts for changes in transcription and enables identification of genes for which changes in translation do not simply parallel changes in mRNA abundance. Using FDR<0.1, sustained high glucose was found to up-regulate 3393 genes and down-regulate 3382 genes (data not shown).

Among genes for which TE was up-regulated by chronic high glucose, pathways related to chromatin organization, RNA splicing, translation, deubiquitination, and M phase of cell cycle were over-represented (FIG. 3H). Among genes for which TE was down-regulated by chronic high glucose, pathways important for beta-cell function including insulin processing, ER-to Golgi transport, glucose metabolism, and TCA cycle were over-represented (FIG. 3I).

Thus, independent of ER stress or change in global protein synthesis rates, sustained high glucose had genome-wide effects on translation. Moreover, in addition to insulin, sustained high glucose down-regulated translation of other genes required for metabolism-coupled insulin secretion.

### Example 4. Nascent Proteomics is an Independent Measure of Glucose-Altered Translation

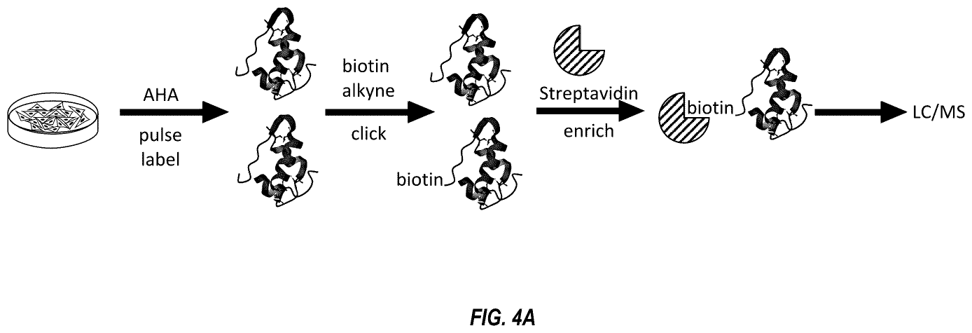

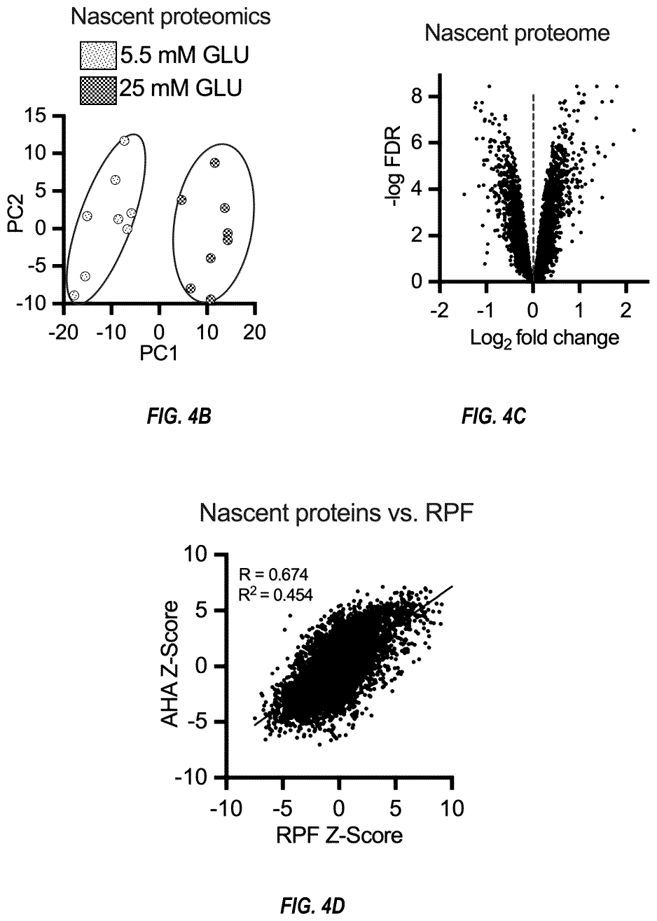

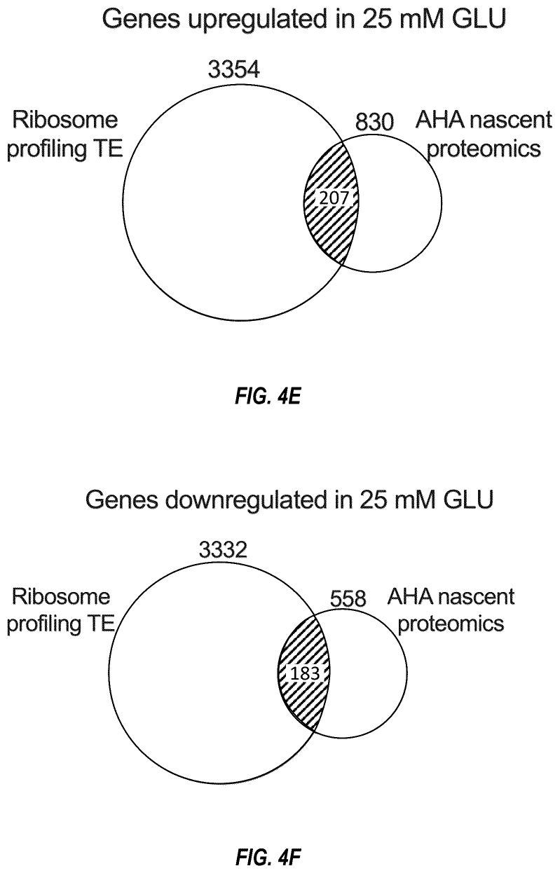

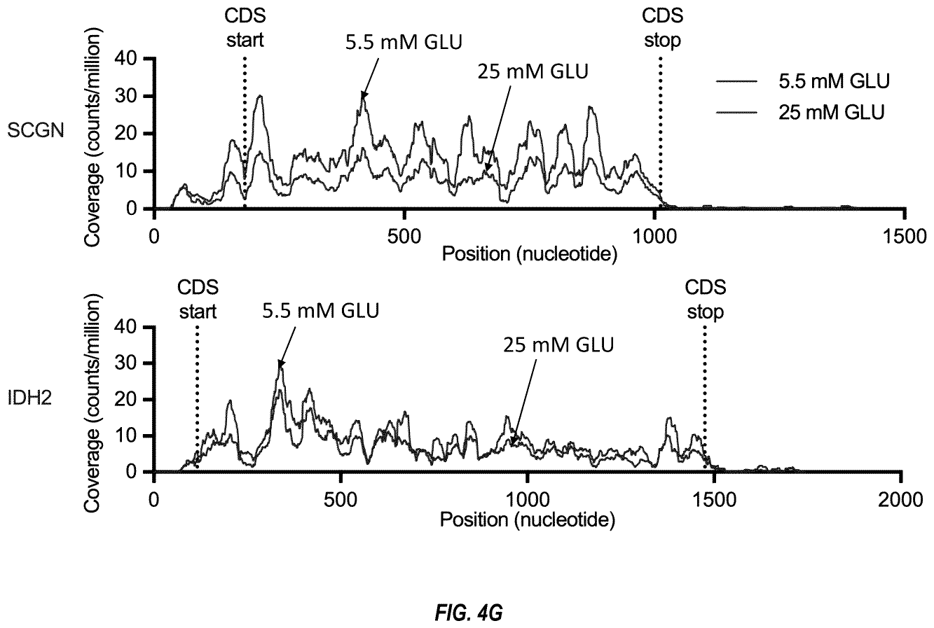

Ribosome profiling analysis in MIN6 cells identified a large number of genes for which translation was affected by sustained high glucose based on the ratio of RPFs to total mRNA. To corroborate and filter these results, an orthogonal method of translation analysis was used in which nascent peptides were pulse labeled with the methionine analogue azidohomoalanine (AHA), click biotin conjugated, and enriched by streptavidin pulldown up front of mass spectrometry-based proteomics (FIG. 4A). Principle component analysis revealed distinct patterns of nascent protein synthesis under the different glucose treatment conditions (FIG. 4B). Sustained high glucose significantly affected new peptide synthesis for many genes, and this correlated well with abundance of RPF sequence (FIG. 4C-4D).

To identify high confidence glucose-driven translation changes, the overlap between nascent proteomics and ribosome profiling TE was evaluated, focusing on genes for which changes in both analyses met FDR<0.1 and 20% log 2 fold. Based on these criteria, 207 genes were upregulated by sustained high glucose and 183 genes were downregulated (FIGS. 4E-4F, Table 1).

TABLE 1
Up-regulated and Down-regulated Genes

| | | | |
|---|---|---|---|
| Symbol | Gene_id | Entrezid | Description |

High Confidence Translationally Up-Regulated Genes

| | | | |
|---|---|---|---|
| Aak1 | ENSMUSG00000057230 | 269774 | AP2 associated kinase 1 [Source: MGI |
| | | | Symbol; Acc: MGI: 1098687] |
| Abcb1a | ENSMUSG00000040584 | 18671 | ATP-binding cassette, sub-family B (MDR/TAP), |
| | | | member 1A [Source: MGI Symbol; Acc: MGI: 97570] |
| Abcb4 | ENSMUSG00000042476 | 18670 | ATP-binding cassette, sub-family B (MDR/TAP), |
| | | | member 4 [Source: MGI Symbol; Acc: MGI: 97569] |
| Aff2 | ENSMUSG00000031189 | 14266 | AF4/FMR2 family, member 2 [Source: MGI |
| | | | Symbol; Acc: MGI: 1202294] |
| Ahdc1 | ENSMUSG00000037692 | 230793 | AT hook, DNA binding motif, containing 1 |
| | | | [Source: MGI Symbol; Acc: MGI: 2444218] |
| Ahsa2 | ENSMUSG00000020288 | 268390 | AHA1, activator of heat shock protein ATPase 2 |
| | | | [Source: MGI Symbol; Acc: MGI: 1916133] |
| Aimp1 | ENSMUSG00000028029 | 13722 | aminoacyl tRNA synthetase complex-interacting |
| | | | multifunctional protein 1 [Source: MGI |
| | | | Symbol; Acc: MGI: 102774] |
| Anks4b | ENSMUSG00000030909 | 72074 | ankyrin repeat and sterile alpha motif domain |
| | | | containing 4B [Source: MGI Symbol; Acc: MGI: 1919324] |
| Apex2 | ENSMUSG00000025269 | 77622 | apurinic/apyrimidinic endonuclease 2 |
| | | | [Source: MGI Symbol; Acc: MGI: 1924872] |
| Arfgap3 | ENSMUSG00000054277 | 66251 | ADP-ribosylation factor GTPase activating |
| | | | protein 3 [Source: MGI Symbol; Acc: MGI: 1913501] |
| Arhgap11a | ENSMUSG00000041219 | 228482 | Rho GTPase activating protein 11A [Source: MGI |
| | | | Symbol; Acc: MGI: 2444300] |
| Aup1 | ENSMUSG00000068328 | 11993 | ancient ubiquitous protein 1 [Source: MGI |
| | | | Symbol; Acc: MGI: 107789] |
| B3galt4 | ENSMUSG00000067370 | 54218 | UDP-Gal: betaGlcNAc beta 1,3-galactosyltransferase, |
| | | | polypeptide 4 [Source: MGI Symbol; Acc: MGI: 1859517] |
| Bcar1 | ENSMUSG00000031955 | 12927 | breast cancer anti-estrogen resistance 1 |
| | | | [Source: MGI Symbol; Acc: MGI: 108091] |
| Bex1 | ENSMUSG00000050071 | 19716 | brain expressed X-linked 1 [Source: MGI |
| | | | Symbol; Acc: MGI: 1328321] |
| Bfar | ENSMUSG00000022684 | 67118 | bifunctional apoptosis regulator [Source: MGI |
| | | | Symbol; Acc: MGI: 1914368] |
| C1qtnf1 | ENSMUSG00000017446 | 56745 | C1q and tumor necrosis factor related protein 1 |
| | | | [Source: MGI Symbol; Acc: MGI: 1919254] |
| Cacfd1 | ENSMUSG00000015488 | 381356 | calcium channel flower domain containing 1 |
| | | | [Source: MGI Symbol; Acc: MGI: 1924317] |
| Calm3 | ENSMUSG00000019370 | 12315 | calmodulin 3 [Source: MGI Symbol; Acc: MGI: 103249] |
| Cbx6 | ENSMUSG00000089715 | 494448 | chromobox 6 [Source: MGI Symbol; Acc: MGI: 3512628] |
| Ccdc167 | ENSMUSG00000024018 | 68597 | coiled-coil domain containing 167 [Source: MGI |
| | | | Symbol; Acc: MGI: 1915847] |
| Ccnd2 | ENSMUSG00000000184 | 12444 | cyclin D2 [Source: MGI Symbol; Acc: MGI: 88314] |
| Cdca2 | ENSMUSG00000048922 | 108912 | cell division cycle associated 2 [Source: MGI |
| | | | Symbol; Acc: MGI: 1919787] |
| Cdca8 | ENSMUSG00000028873 | 52276 | cell division cycle associated 8 [Source: MGI |
| | | | Symbol; Acc: MGI: 1196274] |
| Cdk5rap3 | ENSMUSG00000018669 | 80280 | CDK5 regulatory subunit associated protein 3 |
| | | | [Source: MGI Symbol; Acc: MGI: 1933126] |
| Cfap36 | ENSMUSG00000020462 | 216618 | cilia and flagella associated protein 36 |
| | | | [Source: MGI Symbol; Acc: MGI: 1913994] |
| Cgn | ENSMUSG00000068876 | 70737 | cingulin [Source: MGI Symbol; Acc: MGI: 1927237] |
| Cgref1 | ENSMUSG00000029161 | 68567 | cell growth regulator with EF hand domain 1 |
| | | | [Source: MGI Symbol; Acc: MGI: 1915817] |
| Chgb | ENSMUSG00000027350 | 12653 | chromogranin B [Source: MGI Symbol; Acc: MGI: 88395] |
| Chic1 | ENSMUSG00000031327 | 12212 | cysteine-rich hydrophobic domain 1 [Source: MGI |
| | | | Symbol; Acc: MGI: 1344694] |
| Chid1 | ENSMUSG00000025512 | 68038 | chitinase domain containing 1 [Source: MGI |
| | | | Symbol; Acc: MGI: 1915288] |
| Chpf | ENSMUSG00000032997 | 74241 | chondroitin polymerizing factor [Source: MGI |
| | | | Symbol; Acc: MGI: 106576] |
| Ckap21 | ENSMUSG00000048327 | 70466 | cytoskeleton associated protein 2-like |
| | | | [Source: MGI Symbol; Acc: MGI: 1917716] |
| Ckap4 | ENSMUSG00000046841 | 216197 | cytoskeleton-associated protein 4 [Source: MGI |
| | | | Symbol; Acc: MGI: 2444926] |
| Clgn | ENSMUSG00000002190 | 12745 | calmegin [Source: MGI Symbol; Acc: MGI: 107472] |
| Clip2 | ENSMUSG00000063146 | 269713 | CAP-GLY domain containing linker protein 2 |
| | | | [Source: MGI Symbol; Acc: MGI: 1313136] |
| Clstn2 | ENSMUSG00000032452 | 64085 | calsyntenin 2 [Source: MGI Symbol; Acc: MGI: 1929897] |
| Copa | ENSMUSG00000026553 | 12847 | coatomer protein complex subunit alpha |
| | | | [Source: MGI Symbol; Acc: MGI: 1334462] |
| Copz1 | ENSMUSG00000060992 | 56447 | coatomer protein complex, subunit zeta 1 |
| | | | [Source: MGI Symbol; Acc: MGI: 1929063] |
| Creb3l2 | ENSMUSG00000038648 | 208647 | cAMP responsive element binding protein 3-like 2 |
| | | | [Source: MGI Symbol; Acc: MGI: 2442695] |
| Crybb3 | ENSMUSG00000029352 | 12962 | crystallin, beta B3 [Source: MGI |
| | | | Symbol; Acc: MGI: 102717] |
| Ctsz | ENSMUSG00000016256 | 64138 | cathepsin Z [Source: MGI Symbol; Acc: MGI: 1891190] |
| Cul7 | ENSMUSG00000038545 | 66515 | cullin 7 [Source: MGI Symbol; Acc: MGI: 1913765] |
| Ddrgk1 | ENSMUSG00000068290 | 77006 | DDRGK domain containing 1 [Source: MGI |
| | | | Symbol; Acc: MGI: 1924256] |
| Denr | ENSMUSG00000023106 | 68184 | density-regulated protein [Source: MGI |
| | | | Symbol; Acc: MGI: 1915434] |
| Dhfr | ENSMUSG00000021707 | 13361 | dihydrofolate reductase [Source: MGI |
| | | | Symbol; Acc: MGI: 94890] |
| Dnajc21 | ENSMUSG00000044224 | 78244 | DnaJ heat shock protein family (Hsp40) member |
| | | | C21 [Source: MGI Symbol; Acc: MGI: 1925371] |
| Dnajc3 | ENSMUSG00000022136 | 100037258 | DnaJ heat shock protein family (Hsp40) member |
| | | | C3 [Source: MGI Symbol; Acc: MGI: 107373] |
| Dsg2 | ENSMUSG00000044393 | 13511 | desmoglein 2 [Source: MGI Symbol; Acc: MGI: 1196466] |
| Dusp22 | ENSMUSG00000069255 | 105352 | dual specificity phosphatase 22 [Source: MGI |
| | | | Symbol; Acc: MGI: 1915926] |
| Dusp3 | ENSMUSG00000003518 | 72349 | dual specificity phosphatase 3 (vaccinia virus |
| | | | phosphatase VH1-related) [Source: MGI |
| | | | Symbol; Acc: MGI: 1919599] |
| Dusp4 | ENSMUSG00000031530 | 319520 | dual specificity phosphatase 4 [Source: MGI |
| | | | Symbol; Acc: MGI: 2442191] |
| Dync1i1 | ENSMUSG00000029757 | 13426 | dynein cytoplasmic 1 intermediate chain 1 |
| | | | [Source: MGI Symbol; Acc: MGI: 107743] |
| Edem2 | ENSMUSG00000038312 | 108687 | ER degradation enhancer, mannosidase alpha-like |
| | | | 2 [Source: MGI Symbol; Acc: MGI: 1915540] |
| Eef1g | ENSMUSG00000071644 | 67160 | eukaryotic translation elongation factor 1 gamma |
| | | | [Source: MGI Symbol; Acc: MGI: 1914410] |
| Eif2b2 | ENSMUSG00000004788 | 217715 | eukaryotic translation initiation factor 2B, subunit |
| | | | 2 beta [Source: MGI Symbol; Acc: MGI: 2145118] |
| Eif2b4 | ENSMUSG00000029145 | 13667 | eukaryotic translation initiation factor 2B, subunit |
| | | | 4 delta [Source: MGI Symbol; Acc: MGI: 95300] |
| Eif3b | ENSMUSG00000056076 | 27979 | eukaryotic translation initiation factor 3, subunit B |
| | | | [Source: MGI Symbol; Acc: MGI: 106478] |
| Eif3j2 | ENSMUSG00000043424 | 100042807 | eukaryotic translation initiation factor 3, subunit |
| | | | J2 [Source: MGI Symbol; Acc: MGI: 3704486] |
| Eif4g1 | ENSMUSG00000045983 | 208643 | eukaryotic translation initiation factor 4, gamma 1 |
| | | | [Source: MGI Symbol; Acc: MGI: 2384784] |
| Eif4h | ENSMUSG00000040731 | 22384 | eukaryotic translation initiation factor 4H |
| | | | [Source: MGI Symbol; Acc: MGI: 1341822] |
| Eif5 | ENSMUSG00000021282 | 217869 | eukaryotic translation initiation factor 5 |
| | | | [Source: MGI Symbol; Acc: MGI: 95309] |
| Eif6 | ENSMUSG00000027613 | 16418 | eukaryotic translation initiation factor 6 |
| | | | [Source: MGI Symbol; Acc: MGI: 1196288] |
| Eml3 | ENSMUSG00000071647 | 225898 | echinoderm microtubule associated protein like 3 |
| | | | [Source: MGI Symbol; Acc: MGI: 2387612] |
| Eno2 | ENSMUSG00000004267 | 13807 | enolase 2, gamma neuronal [Source: MGI |
| | | | Symbol; Acc: MGI: 95394] |
| Eno3 | ENSMUSG00000060600 | 13808 | enolase 3, beta muscle [Source: MGI |
| | | | Symbol; Acc: MGI: 95395] |
| Erp29 | ENSMUSG00000029616 | 67397 | endoplasmic reticulum protein 29 [Source: MGI |
| | | | Symbol; Acc: MGI: 1914647] |
| Fam114a1 | ENSMUSG00000029185 | 68303 | family with sequence similarity 114, member A1 |
| | | | [Source: MGI Symbol; Acc: MGI: 1915553] |
| Fam98a | ENSMUSG00000002017 | 72722 | family with sequence similarity 98, member A |
| | | | [Source: MGI Symbol; Acc: MGI: 1919972] |
| Fer1l4 | ENSMUSG00000013338 | 74562 | fer-1-like 4 (C. elegans) [Source: MGI |
| | | | Symbol; Acc: MGI: 1921812] |
| Fkbp11 | ENSMUSG00000003355 | 66120 | FK506 binding protein 11 [Source: MGI |
| | | | Symbol; Acc: MGI: 1913370] |
| Fkbp2 | ENSMUSG00000056629 | 14227 /// | FK506 binding protein 2 [Source: MGI |
| | | 114841036 | Symbol; Acc: MGI: 95542] |
| Fkbp9 | ENSMUSG00000029781 | 27055 | FK506 binding protein 9 [Source: MGI |
| | | | Symbol; Acc: MGI: 1350921] |
| Ftsj3 | ENSMUSG00000020706 | 56095 | FtsJ RNA methyltransferase homolog 3 (E. coli) |
| | | | [Source: MGI Symbol; Acc: MGI: 1860295] |
| Gcnt2 | ENSMUSG00000021360 | 14538 | glucosaminyl (N-acetyl) transferase 2, I-branching |
| | | | enzyme [Source: MGI Symbol; Acc: MGI: 1100870] |
| Glo1 | ENSMUSG00000024026 | 109801 | glyoxalase 1 [Source: MGI Symbol; Acc: MGI: 95742] |
| Golga2 | ENSMUSG00000002546 | 99412 | golgi autoantigen, golgin subfamily a, 2 |
| | | | [Source: MGI Symbol; Acc: MGI: 2139395] |
| Gramd1a | ENSMUSG00000001248 | 52857 | GRAM domain containing 1A [Source: MGI |
| | | | Symbol; Acc: MGI: 105490] |
| Grip1 | ENSMUSG00000034813 | 74053 | glutamate receptor interacting protein 1 |
| | | | [Source: MGI Symbol; Acc: MGI: 1921303] |
| Gsdma | ENSMUSG00000017204 | 57911 | gasdermin A [Source: MGI Symbol; Acc: MGI: 1889509] |
| Gtf2f1 | ENSMUSG00000002658 | 98053 | general transcription factor IIF, polypeptide 1 |
| | | | [Source: MGI Symbol; Acc: MGI: 1923848] |
| Hexim1 | ENSMUSG00000048878 | 192231 | hexamethylene bis-acetamide inducible 1 |
| | | | [Source: MGI Symbol; Acc: MGI: 2385923] |
| Hirip3 | ENSMUSG00000042606 | 233876 | HIRA interacting protein 3 [Source: MGI |
| | | | Symbol; Acc: MGI: 2142364] |
| Hjurp | ENSMUSG00000044783 | 212427 /// | Holliday junction recognition protein |
| | | 381280 | [Source: MGI Symbol; Acc: MGI: 2685821] |
| Hmmr | ENSMUSG00000020330 | 15366 | hyaluronan mediated motility receptor (RHAMM) |
| | | | [Source: MGI Symbol; Acc: MGI: 104667] |
| Hras | ENSMUSG00000025499 | 15461 | Harvey rat sarcoma virus oncogene [Source: MGI |
| | | | Symbol; Acc: MGI: 96224] |
| Hsp90b1 | ENSMUSG00000020048 | 22027 | heat shock protein 90, beta (Grp94), member 1 |
| | | | [Source: MGI Symbol; Acc: MGI: 98817] |
| Ier3ip1 | ENSMUSG00000090000 | 66191 | immediate early response 3 interacting protein 1 |
| | | | [Source: MGI Symbol; Acc: MGI: 1913441] |
| Iffo1 | ENSMUSG00000038271 | 320678 | intermediate filament family orphan 1 |
| | | | [Source: MGI Symbol; Acc: MGI: 2444516] |
| Ift27 | ENSMUSG00000016637 | 67042 | intraflagellar transport 27 [Source: MGI |
| | | | Symbol; Acc: MGI: 1914292] |
| Ikbip | ENSMUSG00000019975 | 67454 | IKBKB interacting protein [Source: MGI |
| | | | Symbol; Acc: MGI: 1914704] |
| Impdh1 | ENSMUSG00000003500 | 23917 | inosine monophosphate dehydrogenase 1 |
| | | | [Source: MGI Symbol; Acc: MGI: 96567] |
| Incenp | ENSMUSG00000024660 | 16319 | inner centromere protein [Source: MGI |
| | | | Symbol; Acc: MGI: 1313288] |
| Irgq | ENSMUSG00000041037 | 210146 | immunity-related GTPase family, Q [Source: MGI |
| | | | Symbol; Acc: MGI: 2667176] |
| Isg20 | ENSMUSG00000039236 | 57444 | interferon-stimulated protein [Source: MGI |
| | | | Symbol; Acc: MGI: 1928895] |
| Kirrel2 | ENSMUSG00000036915 | 243911 | kirre like nephrin family adhesion molecule 2 |
| | | | [Source: MGI Symbol; Acc: MGI: 2442334] |
| Klhdc4 | ENSMUSG00000040263 | 234825 | kelch domain containing 4 [Source: MGI |
| | | | Symbol; Acc: MGI: 2384569] |
| Klhl1 | ENSMUSG00000022076 | 93688 | kelch-like 1 [Source: MGI Symbol; Acc: MGI: 2136335] |
| Krt18 | ENSMUSG00000023043 | 16668 | keratin 18 [Source: MGI Symbol; Acc: MGI: 96692] |
| Larp1b | ENSMUSG00000025762 | 214048 | La ribonucleoprotein domain family, member 1B |
| | | | [Source: MGI Symbol; Acc: MGI: 1914604] |
| Larp4 | ENSMUSG00000023025 | 207214 | La ribonucleoprotein domain family, member 4 |
| | | | [Source: MGI Symbol; Acc: MGI: 2443114] |
| Leng1 | ENSMUSG00000078813 | 69757 | leukocyte receptor cluster (LRC) member 1 |
| | | | [Source: MGI Symbol; Acc: MGI: 1917007] |
| Limch1 | ENSMUSG00000037736 | 77569 | LIM and calponin homology domains 1 |
| | | | [Source: MGI Symbol; Acc: MGI: 1924819] |
| Lmf1 | ENSMUSG00000002279 | 76483 | lipase maturation factor 1 [Source: MGI |
| | | | Symbol; Acc: MGI: 1923733] |
| Lmna | ENSMUSG00000028063 | 16905 | lamin A [Source: MGI Symbol; Acc: MGI: 96794] |
| Lrrc41 | ENSMUSG00000028703 | 230654 | leucine rich repeat containing 41 [Source: MGI |
| | | | Symbol; Acc: MGI: 2441984] |
| Lrrfip2 | ENSMUSG00000032497 | 71268 | leucine rich repeat (in FLII) interacting protein 2 |
| | | | [Source: MGI Symbol; Acc: MGI: 1918518] |
| Luc71 | ENSMUSG00000024188 | 66978 | Luc7-like [Source: MGI Symbol; Acc: MGI: 1914228] |
| Lyar | ENSMUSG00000067367 | 17089 | Ly1 antibody reactive clone [Source: MGI |
| | | | Symbol; Acc: MGI: 107470] |
| Mapre1 | ENSMUSG00000027479 | 13589 | microtubule-associated protein, RP/EB family, |
| | | | member 1 [Source: MGI Symbol; Acc: MGI: 891995] |
| Mapt | ENSMUSG00000018411 | 17762 | microtubule-associated protein tau [Source: MGI |
| | | | Symbol; Acc: MGI: 97180] |
| Mat1a | ENSMUSG00000037798 | 11720 | methionine adenosyltransferase I, alpha |
| | | | [Source: MGI Symbol; Acc: MGI: 88017] |
| Mcl1 | ENSMUSG00000038612 | 17210 | myeloid cell leukemia sequence 1 [Source: MGI |
| | | | Symbol; Acc: MGI: 101769] |
| Mettl1 | ENSMUSG00000006732 | 17299 | methyltransferase like 1 [Source: MGI |
| | | | Symbol; Acc: MGI: 1339986] |
| Mfge8 | ENSMUSG00000030605 | 17304 | milk fat globule-EGF factor 8 protein |
| | | | [Source: MGI Symbol; Acc: MGI: 102768] |
| Mns1 | ENSMUSG00000032221 | 17427 | meiosis-specific nuclear structural protein 1 |
| | | | [Source: MGI Symbol; Acc: MGI: 107933] |
| Mvb12a | ENSMUSG00000031813 | 73711 | multivesicular body subunit 12A [Source: MGI |
| | | | Symbol; Acc: MGI: 1920961] |
| Nans | ENSMUSG00000028334 | 94181 | N-acetylneuraminic acid synthase (sialic acid |
| | | | synthase) [Source: MGI Symbol; Acc: MGI: 2149820] |
| Nap1l4 | ENSMUSG00000059119 | 17955 | nucleosome assembly protein 1-like 4 |
| | | | [Source: MGI Symbol; Acc: MGI: 1316687] |
| Napb | ENSMUSG00000027438 | 17957 | N-ethylmaleimide sensitive fusion protein |
| | | | attachment protein beta [Source: MGI |
| | | | Symbol; Acc: MGI: 104562] |
| Nefh | ENSMUSG00000020396 | 380684 | neurofilament, heavy polypeptide [Source: MGI |
| | | | Symbol; Acc: MGI: 97309] |
| Nfxl1 | ENSMUSG00000072889 | 100978 | nuclear transcription factor, X-box binding-like 1 |
| | | | [Source: MGI Symbol; Acc: MGI: 1923646] |
| Nme1 | ENSMUSG00000037601 | 18102 | NME/NM23 nucleoside diphosphate kinase 1 |
| | | | [Source: MGI Symbol; Acc: MGI: 97355] |
| Nol8 | ENSMUSG00000021392 | 70930 | nucleolar protein 8 [Source: MGI |
| | | | Symbol; Acc: MGI: 1918180] |
| Nolc1 | ENSMUSG00000015176 | 70769 | nucleolar and coiled-body phosphoprotein 1 |
| | | | [Source: MGI Symbol; Acc: MGI: 1918019] |
| Nop56 | ENSMUSG00000027405 | 67134 | NOP56 ribonucleoprotein [Source: MGI |
| | | | Symbol; Acc: MGI: 1914384] |
| Nrcam | ENSMUSG00000020598 | 319504 | neuronal cell adhesion molecule [Source: MGI |
| | | | Symbol; Acc: MGI: 104750] |
| Nsa2 | ENSMUSG00000060739 | 59050 | NSA2 ribosome biogenesis homolog |
| | | | [Source: MGI Symbol; Acc: MGI: 1913883] |
| Os9 | ENSMUSG00000040462 | 216440 | amplified in osteosarcoma [Source: MGI |
| | | | Symbol; Acc: MGI: 1924301] |
| P4ha1 | ENSMUSG00000019916 | 18451 | procollagen-proline, 2-oxoglutarate 4-dioxygenase |
| | | | (proline 4-hydroxylase), alpha 1 polypeptide |
| | | | [Source: MGI Symbol; Acc: MGI: 97463] |
| Pabpc4 | ENSMUSG00000011257 | 230721 | poly(A) binding protein, cytoplasmic 4 |
| | | | [Source: MGI Symbol; Acc: MGI: 2385206] |
| Pappa2 | ENSMUSG00000073530 | 23850 | pappalysin 2 [Source: MGI Symbol; Acc: MGI: 3051647] |
| Pax2 | ENSMUSG00000004231 | 18504 | paired box 2 [Source: MGI Symbol; Acc: MGI: 97486] |
| Pbdc1 | ENSMUSG00000031226 | 67683 | polysaccharide biosynthesis domain containing 1 |
| | | | [Source: MGI Symbol; Acc: MGI: 1914933] |
| Pdap1 | ENSMUSG00000029623 | 231887 | PDGFA associated protein 1 [Source: MGI |
| | | | Symbol; Acc: MGI: 2448536] |
| Pdxdc1 | ENSMUSG00000022680 | 94184 | pyridoxal-dependent decarboxylase domain containing |
| | | | 1 [Source: MGI Symbol; Acc: MGI: 1920909] |
| Phactr1 | ENSMUSG00000054728 | 218194 | phosphatase and actin regulator 1 [Source: MGI |
| | | | Symbol; Acc: MGI: 2659021] |
| Pick1 | ENSMUSG00000068206 | 18693 | protein interacting with C kinase 1 [Source: MGI |
| | | | Symbol; Acc: MGI: 894645] |
| Pik3r1 | ENSMUSG00000041417 | 18708 | phosphoinositide-3-kinase regulatory subunit 1 |
| | | | [Source: MGI Symbol; Acc: MGI: 97583] |
| Plod3 | ENSMUSG00000004846 | 26433 | procollagen-lysine, 2-oxoglutarate 5-dioxygenase |
| | | | 3 [Source: MGI Symbol; Acc: MGI: 1347008] |
| Pmm1 | ENSMUSG00000022474 | 29858 | phosphomannomutase 1 [Source: MGI |
| | | | Symbol; Acc: MGI: 1353418] |
| Pold3 | ENSMUSG00000030726 | 67967 | polymerase (DNA-directed), delta 3, accessory |
| | | | subunit [Source: MGI Symbol; Acc: MGI: 1915217] |
| Poll | ENSMUSG00000025218 | 56626 | polymerase (RNA), lambda [Source: MGI |
| | | | Symbol; Acc: MGI: 1889000] |
| Polr3d | ENSMUSG00000000776 | 67065 | polymerase (RNA) III (DNA directed) polypeptide D |
| | | | [Source: MGI Symbol; Acc: MGI: 1914315] |
| Ppig | ENSMUSG00000042133 | 228005 | peptidyl-prolyl isomerase G (cyclophilin G) |
| | | | [Source: MGI Symbol; Acc: MGI: 2445173] |
| Ppp1r12c | ENSMUSG00000019254 | 232807 | protein phosphatase 1, regulatory subunit 12C |
| | | | [Source: MGI Symbol; Acc: MGI: 1924258] |
| Ppp1r14b | ENSMUSG00000056612 | 18938 | protein phosphatase 1, regulatory inhibitor subunit |
| | | | 14B [Source: MGI Symbol; Acc: MGI: 107682] |
| Prkcsh | ENSMUSG00000003402 | 19089 | protein kinase C substrate 80K-H [Source: MGI |
| | | | Symbol; Acc: MGI: 107877] |
| Prlr | ENSMUSG00000005268 | 19116 | prolactin receptor [Source: MGI Symbol; Acc: MGI: 97763] |
| Prrc1 | ENSMUSG00000024594 | 73137 | proline-rich coiled-coil 1 [Source: MGI |
| | | | Symbol; Acc: MGI: 1916106] |
| Psmc1 | ENSMUSG00000021178 | 19179 | protease (prosome, macropain) 26S subunit, ATPase 1 |
| | | | [Source: MGI Symbol; Acc: MGI: 106054] |
| Reep2 | ENSMUSG00000038555 | 225362 | receptor accessory protein 2 [Source: MGI |
| | | | Symbol; Acc: MGI: 2385070] |
| Rgs9 | ENSMUSG00000020599 | 19739 | regulator of G-protein signaling 9 [Source: MGI |
| | | | Symbol; Acc: MGI: 1338824] |
| Rnf168 | ENSMUSG00000014074 | 70238 | ring finger protein 168 [Source: MGI |
| | | | Symbol; Acc: MGI: 1917488] |
| Rnf187 | ENSMUSG00000020496 | 108660 | ring finger protein 187 [Source: MGI |
| | | | Symbol; Acc: MGI: 1914224] |
| Rpl29 | ENSMUSG00000048758 | 19944 | ribosomal protein L29 [Source: MGI |
| | | | Symbol; Acc: MGI: 99687] |
| Rplp1 | ENSMUSG00000007892 | 56040 | ribosomal protein, large P1 [Source: MGI |
| | | | Symbol; Acc: MGI: 1927099] |
| Rplp2 | ENSMUSG00000025508 | 67186 | ribosomal protein P2 [Source: MGI |
| | | | Symbol; Acc: MGI: 1914436] |
| Rpn2 | ENSMUSG00000027642 | 20014 | ribophorin II [Source: MGI Symbol; Acc: MGI: 98085] |
| Rps19bp1 | ENSMUSG00000051518 | 66538 | ribosomal protein S19 binding protein 1 |
| | | | [Source: MGI Symbol; Acc: MGI: 1913788] |
| Rps21 | ENSMUSG00000039001 | 66481 | ribosomal protein S21 [Source: MGI |
| | | | Symbol; Acc: MGI: 19137371] |
| rps5 | ENSMUSG00000012848 | 20103 | ribosomal protein S5 [Source: MGI |
| | | | Symbol; Acc: MGI: 1097682] |
| Rrbp1 | ENSMUSG00000027422 | 81910 | ribosome binding protein 1 [Source: MGI |
| | | | Symbol; Acc: MGI: 1932395] |
| Rwdd1 | ENSMUSG00000019782 | 66521 | RWD domain containing 1 [Source: MGI |
| | | | Symbol; Acc: MGI: 1913771] |
| Sapcd2 | ENSMUSG00000026955 | 72080 | suppressor APC domain containing 2 |
| | | | [Source: MGI Symbol; Acc: MGI: 1919330] |
| Scfd2 | ENSMUSG00000062110 | 212986 | Sec1 family domain containing 2 [Source: MGI |
| | | | Symbol; Acc: MGI: 2443446] |
| Scyl1 | ENSMUSG00000024941 | 78891 | SCY1-like 1 (S. cerevisiae) [Source: MGI |
| | | | Symbol; Acc: MGI: 1931787] |
| Sec61g | ENSMUSG00000078974 | 20335 | SEC61, gamma subunit [Source: MGI |
| | | | Symbol; Acc: MGI: 1202066] |
| Sez612 | ENSMUSG00000030683 | 233878 | seizure related 6 homolog like 2 [Source: MGI |
| | | | Symbol; Acc: MGI: 2385295] |
| Ska1 | ENSMUSG00000036223 | 66468 | spindle and kinetochore associated complex |
| | | | subunit 1 [Source: MGI Symbol; Acc: MGI: 1913718] |
| Slc16a6 | ENSMUSG00000041920 | 104681 | solute carrier family 16 (monocarboxylic acid |
| | | | transporters), member 6 [Source: MGI |
| | | | Symbol; Acc: MGI: 2144585] |
| Slc26a2 | ENSMUSG00000034320 | 13521 | solute carrier family 26 (sulfate transporter), |
| | | | member 2 [Source: MGI Symbol; Acc: MGI: 892977] |
| Slc35f1 | ENSMUSG00000038602 | 215085 | solute carrier family 35, member F1 [Source: MGI |
| | | | Symbol; Acc: MGI: 2139810] |
| Slc38a10 | ENSMUSG00000061306 | 72055 | solute carrier family 38, member 10 [Source: MGI |
| | | | Symbol; Acc: MGI: 1919305] |
| Slc39a7 | ENSMUSG00000024327 | 14977 | solute carrier family 39 (zinc transporter), member |
| | | | 7 [Source: MGI Symbol; Acc: MGI: 95909] |
| Spg7 | ENSMUSG00000000738 | 234847 | SPG7, paraplegin matrix AAA peptidase subunit |
| | | | [Source: MGI Symbol; Acc: MGI: 2385906] |
| Spock2 | ENSMUSG00000058297 | 94214 | sparc/osteonectin, cwcv and kazal-like domains |
| | | | proteoglycan 2 [Source: MGI Symbol; Acc: MGI: 1891351] |
| Spout1 | ENSMUSG00000039660 | 227695 | SPOUT domain containing methyltransferase 1 |
| | | | [Source: MGI Symbol; Acc: MGI: 106544] |
| Spryd3 | ENSMUSG00000036966 | 223918 | SPRY domain containing 3 [Source: MGI |
| | | | Symbol; Acc: MGI: 2446175] |
| Spty2d1 | ENSMUSG00000049516 | 101685 | SPT2 chromatin protein domain containing 1 |
| | | | [Source: MGI Symbol; Acc: MGI: 2142062] |
| Srp19 | ENSMUSG00000014504 | 66384 | signal recognition particle 19 [Source: MGI |
| | | | Symbol; Acc: MGI: 1913634] |
| Srp72 | ENSMUSG00000036323 | 66661 | signal recognition particle 72 [Source: MGI |
| | | | Symbol; Acc: MGI: 1333795] |
| Ssr1 | ENSMUSG00000021427 | 107513 | signal sequence receptor, alpha [Source: MGI |
| | | | Symbol; Acc: MGI: 105082] |
| Tango6 | ENSMUSG00000041949 | 272538 | transport and golgi organization 6 [Source: MGI |
| | | | Symbol; Acc: MGI: 2142786] |
| Tapbpl | ENSMUSG00000038213 | 213233 | TAP binding protein-like [Source: MGI |
| | | | Symbol; Acc: MGI: 2384853] |
| Tbrg1 | ENSMUSG00000011114 | 21376 | transforming growth factor beta regulated gene 1 |
| | | | [Source: MGI Symbol; Acc: MGI: 1100877] |
| Tcea13 | ENSMUSG00000044550 | 594844 | transcription elongation factor A (SII)-like 3 |
| | | | [Source: MGI Symbol; Acc: MGI: 1913354] |
| Tcof1 | ENSMUSG00000024613 | 21453 | treacle ribosome biogenesis factor 1 [Source: MGI |
| | | | Symbol; Acc: MGI: 892003] |
| Tent5a | ENSMUSG00000032265 | 212943 | terminal nucleotidyltransferase 5A [Source: MGI |
| | | | Symbol; Acc: MGI: 2670964] |
| Timm17a | ENSMUSG00000062580 | 21854 | translocase of inner mitochondrial membrane 17a |
| | | | [Source: MGI Symbol; Acc: MGI: 1343131] |
| Tmed9 | ENSMUSG00000058569 | 67511 | transmembrane p24 trafficking protein 9 |
| | | | [Source: MGI Symbol; Acc: MGI: 1914761] |
| Tmem214 | ENSMUSG00000038828 | 68796 | transmembrane protein 214 [Source: MGI |
| | | | Symbol; Acc: MGI: 1916046] |
| Tpx2 | ENSMUSG00000027469 | 72119 | TPX2, microtubule-associated [Source: MGI |
| | | | Symbol; Acc: MGI: 1919369] |
| Trip11 | ENSMUSG00000021188 | 109181 | thyroid hormone receptor interactor 11 |
| | | | [Source: MGI Symbol; Acc: MGI: 1924393] |
| Ttc1 | ENSMUSG00000041278 | 66827 | tetratricopeptide repeat domain 1 [Source: MGI |
| | | | Symbol; Acc: MGI: 1914077] |
| Tuba4a | ENSMUSG00000026202 | 22145 | tubulin, alpha 4A [Source: MGI Symbol; Acc: MGI: 1095410] |
| Ubr4 | ENSMUSG00000066036 | 69116 | ubiquitin protein ligase E3 component n-recognin |
| | | | 4 [Source: MGI Symbol; Acc: MGI: 1916366] |
| Ubxn4 | ENSMUSG00000026353 | 67812 | UBX domain protein 4 [Source: MGI |
| | | | Symbol; Acc: MGI: 1915062] |
| Ufl1 | ENSMUSG00000040359 | 67490 | UFM1 specific ligase 1 [Source: MGI |
| | | | Symbol; Acc: MGI: 1914740] |
| Upf3b | ENSMUSG00000036572 | 68134 | UPF3 regulator of nonsense transcripts homolog |
| | | | B (yeast) [Source: MGI Symbol; Acc: MGI: 1915384] |
| Usp16 | ENSMUSG00000025616 | 74112 | ubiquitin specific peptidase 16 [Source: MGI |
| | | | Symbol; Acc: MGI: 1921362] |
| Vcp | ENSMUSG00000028452 | 269523 | valosin containing protein [Source: MGI |
| | | | Symbol; Acc: MGI: 99919] |
| Wdr54 | ENSMUSG00000030032 | 75659 | WD repeat domain 54 [Source: MGI |
| | | | Symbol; Acc: MGI: 1922909] |
| Whamm | ENSMUSG00000045795 | 434204 | WAS protein homolog associated with actin, golgi |
| | | | membranes and microtubules [Source: MGI |
| | | | Symbol; Acc: MGI: 2142282] |
| Yipf5 | ENSMUSG00000024487 | 67180 | Yip1 domain family, member 5 [Source: MGI |
| | | | Symbol; Acc: MGI: 1914430] |
| Zc3h12c | ENSMUSG00000035164 | 244871 | zinc finger CCCH type containing 12C |
| | | | [Source: MGI Symbol; Acc: MGI: 3026959] |
| Zc3h15 | ENSMUSG00000027091 | 69082 | zinc finger CCCH-type containing 15 |
| | | | [Source: MGI Symbol; Acc: MGI: 1919747] |

High Confidence Translationally Down-Regulated Genes

| | | | |
|---|---|---|---|
| A1cf | ENSMUSG00000052595 | 69865 | APOBEC1 complementation factor [Source: MGI |
| | | | Symbol; Acc: MGI: 1917115] |
| Aacs | ENSMUSG00000029482 | 78894 | acetoacetyl-CoA synthetase [Source: MGI |
| | | | Symbol; Acc: MGI: 1926144] |
| Acat1 | ENSMUSG00000032047 | 110446 | acetyl-Coenzyme A acetyltransferase 1 |
| | | | [Source: MGI Symbol; Acc: MGI: 87870] |
| Acox1 | ENSMUSG00000020777 | 11430 | acyl-Coenzyme A oxidase 1, palmitoyl |
| | | | [Source: MGI Symbol; Acc: MGI: 1330812] |
| Ago1 | ENSMUSG00000041530 | 236511 | argonaute RISC catalytic subunit 1 [Source: MGI |
| | | | Symbol; Acc: MGI: 2446630] |
| Akr1c12 | ENSMUSG00000021211 | 622402 | aldo-keto reductase family 1, member C12 |
| | | | [Source: MGI Symbol; Acc: MGI: 1351661] |
| Akr1c13 | ENSMUSG00000021213 | 27384 | aldo-keto reductase family 1, member C13 |
| | | | [Source: MGI Symbol; Acc: MGI: 1351662] |
| Aldh6a1 | ENSMUSG00000021238 | 104776 | aldehyde dehydrogenase family 6, subfamily A1 |
| | | | [Source: MGI Symbol; Acc: MGI: 1915077] |
| Ankrd44 | ENSMUSG00000052331 | 329154 | ankyrin repeat domain 44 [Source: MGI |
| | | | Symbol; Acc: MGI: 3045243] |
| Aplp1 | ENSMUSG00000006651 | 11803 | amyloid beta (A4) precursor-like protein 1 |
| | | | [Source: MGI Symbol; Acc: MGI: 88046] |
| App | ENSMUSG00000022892 | 11820 | amyloid beta (A4) precursor protein [Source: MGI |
| | | | Symbol; Acc: MGI: 88059] |
| Arhgap26 | ENSMUSG00000036452 | 71302 | Rho GTPase activating protein 26 [Source: MGI |
| | | | Symbol; Acc: MGI: 1918552] |
| Asah1 | ENSMUSG00000031591 | 11886 | N-acylsphingosine amidohydrolase 1 |
| | | | [Source: MGI Symbol; Acc: MGI: 1277124] |
| Asns | ENSMUSG00000029752 | 27053 | asparagine synthetase [Source: MGI |
| | | | Symbol; Acc: MGI: 1350929] |
| Atm | ENSMUSG00000034218 | 11920 | ataxia telangiectasia mutated [Source: MGI |
| | | | Symbol; Acc: MGI: 107202] |
| Atp2c1 | ENSMUSG00000032570 | 235574 | ATPase, Ca++-sequestering [Source: MGI |
| | | | Symbol; Acc: MGI: 1889008] |
| Atrn | ENSMUSG00000027312 | 11990 | attractin [Source: MGI Symbol; Acc: MGI: 1341628] |
| Atrnl1 | ENSMUSG00000054843 | 226255 | attractin like 1 [Source: MGI Symbol; Acc: MGI: 2147749] |
| Bcor | ENSMUSG00000040363 | 71458 | BCL6 interacting corepressor [Source: MGI |
| | | | Symbol; Acc: MGI: 1918708] |
| Bmi1 | ENSMUSG00000026739 | 12151 | Bmi1 polycomb ring finger oncogene |
| | | | [Source: MGI Symbol; Acc: MGI: 88174] |
| Bphl | ENSMUSG00000038286 | 68021 | biphenyl hydrolase-like (serine hydrolase, breast |
| | | | epithelial mucin-associated antigen) [Source: MGI |
| | | | Symbol; Acc: MGI: 1915271] |
| Brd1 | ENSMUSG00000022387 | 223770 | bromodomain containing 1 [Source: MGI |
| | | | Symbol; Acc: MGI: 1924161] |
| Calcoco1 | ENSMUSG00000023055 | 67488 | calcium binding and coiled coil domain 1 |
| | | | [Source: MGI Symbol; Acc: MGI: 1914738] |
| Casp3 | ENSMUSG00000031628 | 12367 | caspase 3 [Source: MGI Symbol; Acc: MGI: 107739] |
| Cbarp | ENSMUSG00000035640 | 100503659 | calcium channel, voltage-dependent, beta subunit |
| | | | associated regulatory protein [Source: MGI |
| | | | Symbol; Acc: MGI: 1354170] |
| Cdh1 | ENSMUSG00000000303 | 12550 | cadherin 1 [Source: MGI Symbol; Acc: MGI: 88354] |
| Cdkn1b | ENSMUSG00000003031 | 12576 | cyclin-dependent kinase inhibitor 1B |
| | | | [Source: MGI Symbol; Acc: MGI: 104565] |
| Clip3 | ENSMUSG00000013921 | 76686 | CAP-GLY domain containing linker protein 3 |
| | | | [Source: MGI Symbol; Acc: MGI: 1923936] |
| Cnn3 | ENSMUSG00000053931 | 71994 | calponin 3, acidic [Source: MGI |
| | | | Symbol; Acc: MGI: 1919244] |
| Cox15 | ENSMUSG00000040018 | 226139 | cytochrome c oxidase assembly protein 15 |
| | | | [Source: MGI Symbol; Acc: MGI: 1920112] |
| Cox7a2 | ENSMUSG00000032330 | 12866 | cytochrome c oxidase subunit 7A2 [Source: MGI |
| | | | Symbol; Acc: MGI: 1316715] |
| Cpeb1 | ENSMUSG00000025586 | 12877 | cytoplasmic polyadenylation element binding |
| | | | protein 1 [Source: MGI Symbol; Acc: MGI: 108442] |
| Cpq | ENSMUSG00000039007 | 54381 | carboxypeptidase Q [Source: MGI |
| | | | Symbol; Acc: MGI: 1889205] |
| Cpt1a | ENSMUSG00000024900 | 12894 | carnitine palmitoyltransferase 1a, liver |
| | | | [Source: MGI Symbol; Acc: MGI: 1098296] |
| Crebzf | ENSMUSG00000051451 | 233490 | CREB/ATF bZIP transcription factor |
| | | | [Source: MGI Symbol; Acc: MGI: 2675296] |
| Crot | ENSMUSG00000003623 | 74114 | carnitine O-octanoyltransferase [Source: MGI |
| | | | Symbol; Acc: MGI: 1921364] |
| Cryl1 | ENSMUSG00000021947 | 68631 | crystallin, lambda 1 [Source: MGI |
| | | | Symbol; Acc: MGI: 1915881] |
| Csnk2b | ENSMUSG00000024387 | 13001 | casein kinase 2, beta polypeptide [Source: MGI |
| | | | Symbol; Acc: MGI: 88548] |
| Dcaf12l1 | ENSMUSG00000045284 | 245404 | DDB1 and CUL4 associated factor 12-like 1 |
| | | | [Source: MGI Symbol; Acc: MGI: 2444462] |
| Ddr1 | ENSMUSG00000003534 | 12305 | discoidin domain receptor family, member 1 |
| | | | [Source: MGI Symbol; Acc: MGI: 99216] |
| Ddx31 | ENSMUSG00000026806 | 227674 | DEAD/H (Asp-Glu-Ala-Asp/His) box polypeptide |
| | | | 31 [Source: MGI Symbol; Acc: MGI: 2682639] |
| Dennd4c | ENSMUSG00000038024 | 329877 | DENN/MADD domain containing 4C |
| | | | [Source: MGI Symbol; Acc: MGI: 1914769] |
| Dennd5a | ENSMUSG00000035901 | 19347 | DENN/MADD domain containing 5A |
| | | | [Source: MGI Symbol; Acc: MGI: 1201681] |
| Dhx9 | ENSMUSG00000042699 | 13211 | DEAH (Asp-Glu-Ala-His) box polypeptide 9 |
| | | | [Source: MGI Symbol; Acc: MGI: 108177] |
| Disp2 | ENSMUSG00000040035 | 214240 | dispatched RND transporter family member 2 |
| | | | [Source: MGI Symbol; Acc: MGI: 2388733] |
| Dnttip1 | ENSMUSG00000017299 | 76233 | deoxynucleotidyltransferase, terminal, interacting |
| | | | protein 1 [Source: MGI Symbol; Acc: MGI: 1923483] |
| Dpp7 | ENSMUSG00000026958 | 83768 | dipeptidylpeptidase 7 [Source: MGI |
| | | | Symbol; Acc: MGI: 1933213] |
| Dtd1 | ENSMUSG00000027430 | 66044 | D-tyrosy1-tRNA deacylase 1 [Source: MGI |
| | | | Symbol; Acc: MGI: 1913294] |
| Ech1 | ENSMUSG00000053898 | 51798 | enoyl coenzyme A hydratase 1, peroxisomal |
| | | | [Source: MGI Symbol; Acc: MGI: 1858208] |
| Enpp5 | ENSMUSG00000023960 | 83965 | ectonucleotide pyrophosphatase/phosphodiesterase 5 |
| | | | [Source: MGI Symbol; Acc: MGI: 1933830] |
| Epb41l3 | ENSMUSG00000024044 | 13823 | erythrocyte membrane protein band 4.1 like 3 |
| | | | [Source: MGI Symbol; Acc: MGI: 103008] |
| Erap1 | ENSMUSG00000021583 | 80898 | endoplasmic reticulum aminopeptidase 1 |
| | | | [Source: MGI Symbol; Acc: MGI: 1933403] |
| Fam151a | ENSMUSG00000034871 | 230579 | family with sequence simliarity 151, member A |
| | | | [Source: MGI Symbol; Acc: MGI: 2657115] |
| Fam172a | ENSMUSG00000064138 | 68675 | family with sequence similarity 172, member A |
| | | | [Source: MGI Symbol; Acc: MGI: 1915925] |
| Flcn | ENSMUSG00000032633 | 216805 | folliculin [Source: MGI Symbol; Acc: MGI: 2442184] |
| Galc | ENSMUSG00000021003 | 14420 | galactosylceramidase [Source: MGI |
| | | | Symbol; Acc: MGI: 95636] |
| Ghitm | ENSMUSG00000041028 | 66092 | growth hormone inducible transmembrane protein |
| | | | [Source: MGI Symbol; Acc: MGI: 1913342] |
| Gmpr | ENSMUSG00000000253 | 66355 | guanosine monophosphate reductase [Source: MGI |
| | | | Symbol; Acc: MGI: 1913605] |
| Gnb1 | ENSMUSG00000029064 | 14688 | guanine nucleotide binding protein (G protein), |
| | | | beta 1 [Source: MGI Symbol; Acc: MGI: 95781] |
| Gnpda1 | ENSMUSG00000052102 | 26384 | glucosamine-6-phosphate deaminase 1 |
| | | | [Source: MGI Symbol; Acc: MGI: 1347054] |
| Gprasp1 | ENSMUSG00000043384 | 67298 | G protein-coupled receptor associated sorting |
| | | | protein 1 [Source: MGI Symbol; Acc: MGI: 1917418] |
| Grcc10 | ENSMUSG00000072772 | 14790 | gene rich cluster, C10 gene [Source: MGI |
| | | | Symbol; Acc: MGI: 1315201] |
| Grn | ENSMUSG00000034708 | 14824 | granulin [Source: MGI Symbol; Acc: MGI: 95832] |
| Hivep1 | ENSMUSG00000021366 | 110521 | human immunodeficiency virus type I enhancer |
| | | | binding protein 1 [Source: MGI Symbol; Acc: MGI: 96100] |
| Hmgcr | ENSMUSG00000021670 | 15357 | 3-hydroxy-3-methylglutaryl-Coenzyme A |
| | | | reductase [Source: MGI Symbol; Acc: MGI: 96159] |
| Hmox1 | ENSMUSG00000005413 | 15368 | heme oxygenase 1 [Source: MGI Symbol; Acc: MGI: 96163] |
| Hnrnpa0 | ENSMUSG00000007836 | 77134 | heterogeneous nuclear ribonucleoprotein A0 |
| | | | [Source: MGI Symbol; Acc: MGI: 1924384] |
| Hnrnpa1 | ENSMUSG00000046434 | 15382 | heterogeneous nuclear ribonucleoprotein A1 |
| | | | [Source: MGI Symbol; Acc: MGI: 104820] |
| Hnrnpab | ENSMUSG00000020358 | 15384 | heterogeneous nuclear ribonucleoprotein A/B |
| | | | [Source: MGI Symbol; Acc: MGI: 1330294] |
| Hnrnpf | ENSMUSG00000042079 | 98758 | heterogeneous nuclear ribonucleoprotein F |
| | | | [Source: MGI Symbol; Acc: MGI: 2138741] |
| Hnrnpk | ENSMUSG00000021546 | 15387 | heterogeneous nuclear ribonucleoprotein K |
| | | | [Source: MGI Symbol; Acc: MGI: 99894] |
| Hnrnpl | ENSMUSG00000015165 | 15388 | heterogeneous nuclear ribonucleoprotein L |
| | | | [Source: MGI Symbol; Acc: MGI: 104816] |
| Hook2 | ENSMUSG00000052566 | 170833 | hook microtubule tethering protein 2 [Source: MGI |
| | | | Symbol; Acc: MGI: 2181664] |
| Hsbp1 | ENSMUSG00000031839 | 68196 | heat shock factor binding protein 1 [Source: MGI |
| | | | Symbol; Acc: MGI: 1915446] |
| Hsdl2 | ENSMUSG00000028383 | 72479 | hydroxysteroid dehydrogenase like 2 |
| | | | [Source: MGI Symbol; Acc: MGI: 1919729] |
| Hspa12a | ENSMUSG00000025092 | 73442 | heat shock protein 12A [Source: MGI |
| | | | Symbol; Acc: MGI: 1920692] |
| Iah1 | ENSMUSG00000062054 | 67732 | isoamyl acetate-hydrolyzing esterase 1 homolog |
| | | | [Source: MGI Symbol; Acc: MGI: 1914982] |
| Idh2 | ENSMUSG00000030541 | 269951 | isocitrate dehydrogenase 2 (NADP+), |
| | | | mitochondrial [Source: MGI Symbol; Acc: MGI: 96414] |
| Igsf8 | ENSMUSG00000038034 | 140559 | immunoglobulin superfamily, member 8 |
| | | | [Source: MGI Symbol; Acc: MGI: 2154090] |
| Ins1 | ENSMUSG00000035804 | 16333 | insulin I [Source: MGI Symbol; Acc: MGI: 96572] |
| Insrr | ENSMUSG00000005640 | 23920 | insulin receptor-related receptor [Source: MGI |
| | | | Symbol; Acc: MGI: 1346037] |
| Itgb8 | ENSMUSG00000025321 | 320910 | integrin beta 8 [Source: MGI Symbol; Acc: MGI: 1338035] |
| Jmjd1c | ENSMUSG00000037876 | 108829 | jumonji domain containing 1C [Source: MGI |
| | | | Symbol; Acc: MGI: 1918614] |
| Jup | ENSMUSG00000001552 | 16480 | junction plakoglobin [Source: MGI |
| | | | Symbol; Acc: MGI: 96650] |
| Kat7 | ENSMUSG00000038909 | 217127 | K(lysine) acetyltransferase 7 [Source: MGI |
| | | | Symbol; Acc: MGI: 2182799] |
| Kdm3a | ENSMUSG00000053470 | 104263 | lysine (K)-specific demethylase 3A [Source: MGI |
| | | | Symbol; Acc: MGI: 98847] |
| Khdrbs1 | ENSMUSG00000028790 | 20218 | KH domain containing, RNA binding, signal transduction |
| | | | associated 1 [Source: MGI Symbol; Acc: MGI: 893579] |
| Khdrbs3 | ENSMUSG00000022332 | 13992 | KH domain containing, RNA binding, signal |
| | | | transduction associated 3 [Source: MGI |
| | | | Symbol; Acc: MGI: 1313312] |
| Lamp2 | ENSMUSG00000016534 | 16784 | lysosomal-associated membrane protein 2 |
| | | | [Source: MGI Symbol; Acc: MGI: 96748] |
| Man2b1 | ENSMUSG00000005142 | 17159 | mannosidase 2, alpha B1 [Source: MGI |
| | | | Symbol; Acc: MGI: 107286] |
| Maob | ENSMUSG00000040147 | 109731 | monoamine oxidase B [Source: MGI |
| | | | Symbol; Acc: MGI: 96916] |
| Map3k15 | ENSMUSG00000031303 | 270672 | mitogen-activated protein kinase kinase kinase 15 |
| | | | [Source: MGI Symbol; Acc: MGI: 2448588] |
| Map4 | ENSMUSG00000032479 | 17758 | microtubule-associated protein 4 [Source: MGI |
| | | | Symbol; Acc: MGI: 97178] |
| Marchf5 | ENSMUSG00000023307 | 69104 | membrane associated ring-CH-type finger 5 |
| | | | [Source: MGI Symbol; Acc: MGI: 1915207] |
| Mast1 | ENSMUSG00000053693 | 56527 | microtubule associated serine/threonine kinase 1 |
| | | | [Source: MGI Symbol; Acc: MGI: 1861901] |
| Mbtps1 | ENSMUSG00000031835 | 56453 | membrane-bound transcription factor peptidase, |
| | | | site 1 [Source: MGI Symbol; Acc: MGI: 1927235] |
| Mccc1 | ENSMUSG00000027709 | 72039 | methylcrotonoyl-Coenzyme A carboxylase 1 |
| | | | (alpha) [Source: MGI Symbol; Acc: MGI: 1919289] |
| Muc4 | ENSMUSG00000079620 | 140474 | mucin 4 [Source: MGI Symbol; Acc: MGI: 2153525] |
| Myo1b | ENSMUSG00000018417 | 17912 | myosin IB [Source: MGI Symbol; Acc: MGI: 107752] |
| N4bp1 | ENSMUSG00000031652 | 80750 | NEDD4 binding protein 1 [Source: MGI |
| | | | Symbol; Acc: MGI: 2136825] |
| Naaa | ENSMUSG00000029413 | 67111 | N-acylethanolamine acid amidase [Source: MGI |
| | | | Symbol; Acc: MGI: 1914361] |
| Ncoa2 | ENSMUSG00000005886 | 17978 | nuclear receptor coactivator 2 [Source: MGI |
| | | | Symbol; Acc: MGI: 1276533] |
| Ncoa5 | ENSMUSG00000039804 | 228869 | nuclear receptor coactivator 5 [Source: MGI |
| | | | Symbol; Acc: MGI: 2385165] |
| Nol4 | ENSMUSG00000041923 | 319211 | nucleolar protein 4 [Source: MGI |
| | | | Symbol; Acc: MGI: 2441684] |
| Nr1d2 | ENSMUSG00000021775 | 353187 | nuclear receptor subfamily 1, group D, member 2 |
| | | | [Source: MGI Symbol; Acc: MGI: 2449205] |
| Nudt11 | ENSMUSG00000073295 | 58242 | nudix (nucleoside diphosphate linked moiety X)- |
| | | | type motif 11 [Source: MGI Symbol; Acc: MGI: 1930957] |
| Ogdh1 | ENSMUSG00000021913 | 239017 | oxoglutarate dehydrogenase-like [Source: MGI |
| | | | Symbol; Acc: MGI: 3616088] |
| Pcca | ENSMUSG00000041650 | 110821 | propionyl-Coenzyme A carboxylase, alpha |
| | | | polypeptide [Source: MGI Symbol; Acc: MGI: 97499] |
| Phf10 | ENSMUSG00000023883 | 72057 | PHD finger protein 10 [Source: MGI |
| | | | Symbol; Acc: MGI: 1919307] |
| Phf2 | ENSMUSG00000038025 | 18676 | PHD finger protein 2 [Source: MGI |
| | | | Symbol; Acc: MGI: 1338034] |
| Phgdh | ENSMUSG00000053398 | 236539 | 3-phosphoglycerate dehydrogenase [Source: MGI |
| | | | Symbol; Acc: MGI: 1355330] |
| Pi4ka | ENSMUSG00000041720 | 224020 | phosphatidylinositol 4-kinase alpha [Source: MGI |
| | | | Symbol; Acc: MGI: 2448506] |
| Pja1 | ENSMUSG00000034403 | 18744 | praja ring finger ubiquitin ligase 1 [Source: MGI |
| | | | Symbol; Acc: MGI: 1101765] |
| Plekha7 | ENSMUSG00000045659 | 233765 | pleckstrin homology domain containing, family A |
| | | | member 7 [Source: MGI Symbol; Acc: MGI: 2445094] |
| Ppip5k1 | ENSMUSG00000033526 | 327655 | diphosphoinositol pentakisphosphate kinase 1 |
| | | | [Source: MGI Symbol; Acc: MGI: 2443281] |
| Prnp | ENSMUSG00000079037 | 19122 | prion protein [Source: MGI Symbol; Acc: MGI: 97769] |
| Psap | ENSMUSG00000004207 | 19156 | prosaposin [Source: MGI Symbol; Acc: MGI: 97783] |
| Psat1 | ENSMUSG00000024640 | 107272 | phosphoserine aminotransferase 1 [Source: MGI |
| | | | Symbol; Acc: MGI: 2183441] |
| Pspc1 | ENSMUSG00000021938 | 66645 | paraspeckle protein 1 [Source: MGI |
| | | | Symbol; Acc: MGI: 1913895] |
| Psph | ENSMUSG00000029446 | 100678 | phosphoserine phosphatase [Source: MGI |
| | | | Symbol; Acc: MGI: 97788] |
| Ptbp1 | ENSMUSG00000006498 | 19205 | polypyrimidine tract binding protein 1 |
| | | | [Source: MGI Symbol; Acc: MGI: 97791] |
| Ptbp3 | ENSMUSG00000028382 | 230257 | polypyrimidine tract binding protein 3 |
| | | | [Source: MGI Symbol; Acc: MGI: 1923334] |
| Ptk2b | ENSMUSG00000059456 | 19229 | PTK2 protein tyrosine kinase 2 beta [Source: MGI |
| | | | Symbol; Acc: MGI: 104908] |
| Rap1gap | ENSMUSG00000041351 | 110351 | Rap1 GTPase-activating protein [Source: MGI |
| | | | Symbol; Acc: MGI: 109338] |
| Rasa3 | ENSMUSG00000031453 | 19414 | RAS p21 protein activator 3 [Source: MGI |
| | | | Symbol; Acc: MGI: 1197013] |
| Rbm22 | ENSMUSG00000024604 | 66810 | RNA binding motif protein 22 [Source: MGI |
| | | | Symbol; Acc: MGI: 1914060] |
| Rbm45 | ENSMUSG00000042369 | 241490 | RNA binding motif protein 45 [Source: MGI |
| | | | Symbol; Acc: MGI: 2387367] |
| Rbmx | ENSMUSG00000031134 | 19655 | RNA binding motif protein, X chromosome |
| | | | [Source: MGI Symbol; Acc: MGI: 1343044] |
| Reps2 | ENSMUSG00000040855 | 194590 | RALBP1 associated Eps domain containing |
| | | | protein 2 [Source: MGI Symbol; Acc: MGI: 2663511] |
| Resp18 | ENSMUSG00000033061 | 19711 | regulated endocrine-specific protein 18 |
| | | | [Source: MGI Symbol; Acc: MGI: 1098222] |
| Ret | ENSMUSG00000030110 | 19713 | ret proto-oncogene [Source: MGI |
| | | | Symbol; Acc: MGI: 97902] |
| Rfx6 | ENSMUSG00000019900 | 320995 | regulatory factor X, 6 [Source: MGI |
| | | | Symbol; Acc: MGI: 2445208] |
| Rictor | ENSMUSG00000050310 | 78757 | RPTOR independent companion of MTOR, |
| | | | complex 2 [Source: MGI Symbol; Acc: MGI: 1926007] |
| Rnf213 | ENSMUSG00000070327 | 672511 | ring finger protein 213 [Source: MGI |
| | | | Symbol; Acc: MGI: 1289196] |
| Sbf2 | ENSMUSG00000038371 | 319934 | SET binding factor 2 [Source: MGI |
| | | | Symbol; Acc: MGI: 1921831] |
| Scd1 | ENSMUSG00000037071 | 20249 | stearoyl-Coenzyme A desaturase 1 [Source: MGI |
| | | | Symbol; Acc: MGI: 98239] |
| Scgn | ENSMUSG00000021337 | 214189 | secretagogin, EF-hand calcium binding protein |
| | | | [Source: MGI Symbol; Acc: MGI: 2384873] |
| Scpep1 | ENSMUSG00000000278 | 74617 | serine carboxypeptidase 1 [Source: MGI |
| | | | Symbol; Acc: MGI: 1921867] |
| Sesn3 | ENSMUSG00000032009 | 75747 | sestrin 3 [Source: MGI Symbol; Acc: MGI: 1922997] |
| Sez61 | ENSMUSG00000058153 | 56747 | seizure related 6 homolog like [Source: MGI |
| | | | Symbol; Acc: MGI: 1935121] |
| Sgpl1 | ENSMUSG00000020097 | 20397 | sphingosine phosphate lyase 1 [Source: MGI |
| | | | Symbol; Acc: MGI: 1261415] |
| Sh3bgrl | ENSMUSG00000031246 | 56726 | SH3-binding domain glutamic acid-rich protein |
| | | | like [Source: MGI Symbol; Acc: MGI: 1930849] |
| Shmt2 | ENSMUSG00000025403 | 108037 | serine hydroxymethyltransferase 2 (mitochondrial) |
| | | | [Source: MGI Symbol; Acc: MGI: 1277989] |
| Slc1a4 | ENSMUSG00000020142 | 55963 | solute carrier family 1 (glutamate/neutral amino |
| | | | acid transporter), member 4 [Source: MGI |
| | | | Symbol; Acc: MGI: 2135601] |
| Slc25a51 | ENSMUSG00000045973 | 230125 | solute carrier family 25, member 51 [Source: MGI |
| | | | Symbol; Acc: MGI: 2684984] |
| Slc2a2 | ENSMUSG00000027690 | 20526 | solute carrier family 2 (facilitated glucose |
| | | | transporter), member 2 [Source: MGI |
| | | | Symbol; Acc: MGI: 1095438] |
| Slc30a8 | ENSMUSG00000022315 | 239436 | solute carrier family 30 (zinc transporter), member |
| | | | 8 [Source: MGI Symbol; Acc: MGI: 2442682] |
| Slc44a1 | ENSMUSG00000028412 | 100434 | solute carrier family 44, member 1 [Source: MGI |
| | | | Symbol; Acc: MGI: 2140592] |
| Slc6a15 | ENSMUSG00000019894 | 103098 | solute carrier family 6 (neurotransmitter transporter), |
| | | | member 15 [Source: MGI Symbol; Acc: MGI: 2143484] |
| Slc7a2 | ENSMUSG00000031596 | 11988 | solute carrier family 7 (cationic amino acid |
| | | | transporter, y+ system), member 2 [Source: MGI |
| | | | Symbol; Acc: MGI: 99828] |
| Slc7a5 | ENSMUSG00000040010 | 20539 | solute carrier family 7 (cationic amino acid |
| | | | transporter, y+ system), member 5 [Source: MGI |
| | | | Symbol; Acc: MGI: 1298205] |
| Slc7a8 | ENSMUSG00000022180 | 50934 | solute carrier family 7 (cationic amino acid |
| | | | transporter, y+ system), member 8 [Source: MGI |
| | | | Symbol; Acc: MGI: 1355323] |
| Slf2 | ENSMUSG00000036097 | 226151 | SMC5-SMC6 complex localization factor 2 |
| | | | [Source: MGI Symbol; Acc: MGI: 1924968] |
| Snrpa | ENSMUSG00000061479 | 53607 | small nuclear ribonucleoprotein polypeptide A |
| | | | [Source: MGI Symbol; Acc: MGI: 1855690] |
| Snrpc | ENSMUSG00000024217 | 20630 | U1 small nuclear ribonucleoprotein C |
| | | | [Source: MGI Symbol; Acc: MGI: 109489] |
| Spata13 | ENSMUSG00000021990 | 219140 | spermatogenesis associated 13 [Source: MGI |
| | | | Symbol; Acc: MGI: 104838] |
| Sult1d1 | ENSMUSG00000029273 | 53315 | sulfotransferase family 1D, member 1 |
| | | | [Source: MGI Symbol; Acc: MGI: 1926341] |
| Syne1 | ENSMUSG00000096054 | 64009 | spectrin repeat containing, nuclear envelope 1 |
| | | | [Source: MGI Symbol; Acc: MGI: 1927152] |
| Syt7 | ENSMUSG00000024743 | 54525 | synaptotagmin VII [Source: MGI |
| | | | Symbol; Acc: MGI: 1859545] |
| Tbc1d8 | ENSMUSG00000003134 | 54610 | TBC1 domain family, member 8 [Source: MGI |
| | | | Symbol; Acc: MGI: 1927225] |
| Tcpl1l2 | ENSMUSG00000020034 | 216198 | t-complex 11 (mouse) like 2 [Source: MGI |
| | | | Symbol; Acc: MGI: 2444679] |
| Tfrc | ENSMUSG00000022797 | 22042 | transferrin receptor [Source: MGI |
| | | | Symbol; Acc: MGI: 98822] |
| Tmbim6 | ENSMUSG00000023010 | 110213 | transmembrane BAX inhibitor motif containing 6 |
| | | | [Source: MGI Symbol; Acc: MGI: 99682] |
| Tmem131l | ENSMUSG00000033767 | 229473 | transmembrane 131 like [Source: MGI |
| | | | Symbol; Acc: MGI: 2443399] |
| Trappc9 | ENSMUSG00000047921 | 76510 | trafficking protein particle complex 9 |
| | | | [Source: MGI Symbol; Acc: MGI: 1923760] |
| Tshz3 | ENSMUSG00000021217 | 243931 | teashirt zinc finger family member 3 [Source: MGI |
| | | | Symbol; Acc: MGI: 2442819] |
| Ttc39a | ENSMUSG00000028555 | 230603 | tetratricopeptide repeat domain 39A [Source: MGI |
| | | | Symbol; Acc: MGI: 2444350] |
| Tuft1 | ENSMUSG00000005968 | 22156 | tuftelin 1 [Source: MGI Symbol; Acc: MGI: 109572] |
| Txndc16 | ENSMUSG00000021830 | 70561 | thioredoxin domain containing 16 [Source: MGI |
| | | | Symbol; Acc: MGI: 1917811] |
| Uhmk1 | ENSMUSG00000026667 | 16589 | U2AF homology motif (UHM) kinase 1 |
| | | | [Source: MGI Symbol; Acc: MGI: 1341908] |
| Upb1 | ENSMUSG00000033427 | 103149 | ureidopropionase, beta [Source: MGI |
| | | | Symbol; Acc: MGI: 2143535] |
| Usp45 | ENSMUSG00000040455 | 77593 | ubiquitin specific petidase 45 [Source: MGI |
| | | | Symbol; Acc: MGI: 101850] |
| Vat1l | ENSMUSG00000046844 | 270097 | vesicle amine transport protein 1 like |
| | | | [Source: MGI Symbol; Acc: MGI: 2142534] |
| Vld1r | ENSMUSG00000024924 | 22359 | very low density lipoprotein receptor |
| | | | [Source: MGI Symbol; Acc: MGI: 98935] |
| Vps41 | ENSMUSG00000041236 | 218035 | VPS41 HOPS complex subunit [Source: MGI |
| | | | Symbol; Acc: MGI: 1929215] |
| Vwa5a | ENSMUSG00000023186 | 67776 | von Willebrand factor A domain containing 5A |
| | | | [Source: MGI Symbol; Acc: MGI: 1915026] |
| Washc5 | ENSMUSG00000022350 | 223593 | WASH complex subunit 5 [Source: MGI |
| | | | Symbol; Acc: MGI: 2146110] |
| Wbp2 | ENSMUSG00000034341 | 22378 | WW domain binding protein 2 [Source: MGI |
| | | | Symbol; Acc: MGI: 104709] |
| Wdr6 | ENSMUSG00000066357 | 83669 | WD repeat domain 6 [Source: MGI |
| | | | Symbol; Acc: MGI: 1930140] |
| Xrcc5 | ENSMUSG00000026187 | 22596 | X-ray repair complementing defective repair in |
| | | | Chinese hamster cells 5 [Source: MGI |
| | | | Symbol; Acc: MGI: 104517] |
| Zcchc18 | ENSMUSG00000031428 | 66995 | zinc finger, CCHC domain containing 18 |
| | | | [Source: MGI Symbol; Acc: MGI: 1914245] |
| Zdhhc2 | ENSMUSG00000039470 | 70546 | zinc finger, DHHC domain containing 2 |
| | | | [Source: MGI Symbol; Acc: MGI: 1923452] |
| Zmym2 | ENSMUSG00000021945 | 76007 | zinc finger, MYM-type 2 [Source: MGI |
| | | | Symbol; Acc: MGI: 1923257] |

Given that sustained high glucose down-regulates GSIS and insulin translation, a focus was on down-regulated proteins known to function in insulin production and metabolism-coupled insulin secretion (Table 2). RPF gene coverage analysis revealed no evidence that translational downregulation resulted from selection of new upstream open reading frames or pausing under sustained high glucose conditions (FIG. 4G).

TABLE 2
Insulin synthesis/secretion genes translationally downregulated by sustained high glucose*

| | | Ribosome | | | |
|---|---|---|---|---|---|
| Gene | | profiling TE | | Nascent Proteomics | |
| symbol | Role | log2FC | −logFDR | log2FC | −logFDR |
| Idh2 | Insulin secretion through reductive TCA flux | −1.99 | 3.61 | −0.54 | 6.22 |
| Igf2 | Autocrine signaling promotes insulin secretion | −1.34 | 2.29 | nd | nd |
| Ins1 | Insulin gene | −0.77 | 1.14 | −0.72 | 3.61 |
| Ins2 | Insulin gene | −1.45 | 2.29 | # | # |
| Pfkfb3 | Potentiates GSIS through regulation of glucokinase | −2.43 | 4.54 | nd | nd |
| Scgn | Ca++ and insulin binding protein that enhances GSIS | −0.99 | 1.79 | −0.85 | 4.86 |
| Slc2a2 | Transports glucose for metabolism-coupled insulin secretion | −1.03 | 2.67 | −0.45 | 1.44 |
| Slc30a8 | Transports Zn++ into insulin secretory granules | −1.16 | 3.20 | −0.36 | 2.90 |
| Vps41 | Endolysosomal protein in insulin secretory granule formation | −1.26 | 3.05 | −0.41 | 4.05 |

*Sustained high glucose decreases translation efficiency and nascent peptides by 20% or more with FDR < 0.1.
nd, not detected,
# detection limited by peptide overlap with Ins1

SCGN (secretagogin) enhances second phase insulin secretion, and its knockdown impairs GSIS (23, 24). VPS41, a component of the homotypic fusion and vacuole protein sorting complex, and SLC30A8, which transports zinc into insulin granules, are both required for optimal GSIS (25, 26). SLC2A2, the plasma membrane glucose transporter in rodent beta-cells, and IDH2, which functions in reductive flux of glutamine to citrate in the mitochondria, are critical for metabolism-coupled insulin secretion (27, 28). While PFKFB3 and IGF2 were not detected in nascent proteomics, they were significantly downregulated by high glucose in ribosome profiling and were included in further analyses given established roles in potentiating insulin secretion (29, 30).

### Example 5. Validation of Translational Changes and Impact on Steady State Protein Abundance

To confirm these chronic glucose-induced translation changes, the ratio of mRNA associated was quantified with actively translating polysomes relative to total mRNA as a measure of TE in MIN6 cells incubated in 25 vs. 5.5 mM glucose for 24 hours. Sustained high glucose decreased TE of Ins1, Ins2, Scgn, Slc2a2, Pfkfb3, Slc30a8, Vps41, Idh2, and Igf2 (FIG. 5A). Actb and Tubg1 were unchanged by high glucose, consistent with lack of change in nascent protein. To determine whether changes in TE impacted protein levels, steady-state protein abundance was measured in lysates of MIN6 cells treated with 25 vs. 5.5 mM glucose. Sustained high glucose significantly decreased cellular content of SCGN, SLC2A2, PFKFB3, SLC30A8, VPS41, IDH2, and IGF2 (FIG. 5B).

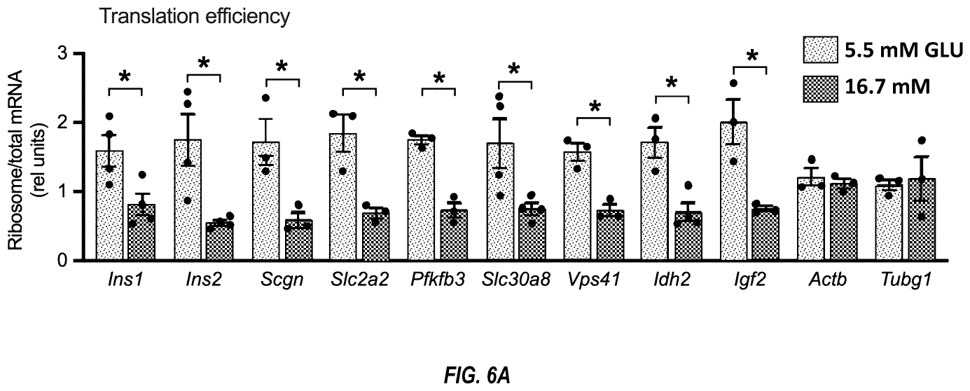

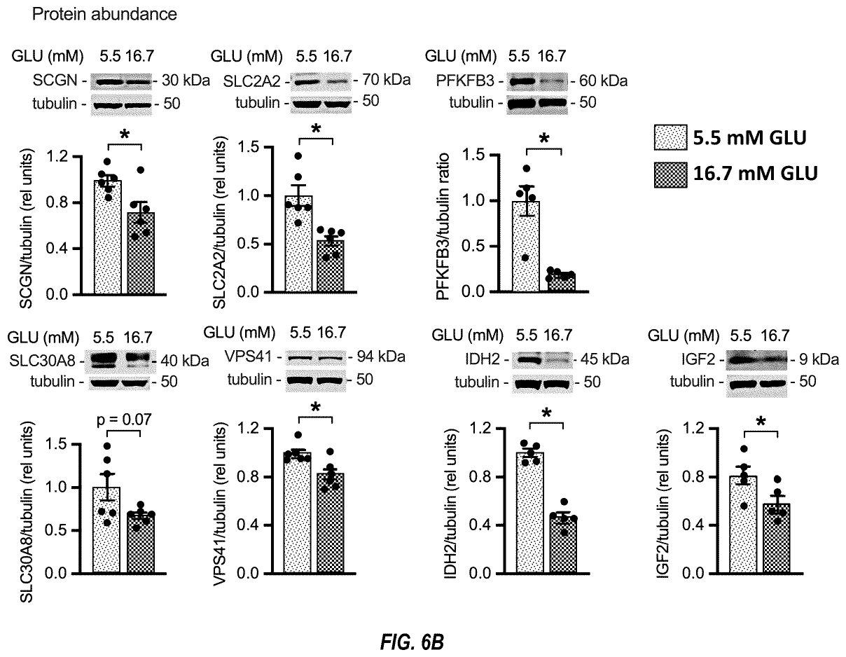

Although MIN6 cells were an important tool for technically challenging high-throughput discovery studies, these cells replicate rapidly, grow dispersed in cell culture, and lack complex cellular make-up and architecture of islets. The findings were next validated in isolated rat islets using the conditions as in FIG. 1A established above that impair glucose stimulated insulin translation and secretion. Given the large amount of tissue needed to collect actively translating polysomes and limited number of islets, ribosome-associated (rather than polysome) mRNA relative to total mRNA was quantified as a measure of TE. Sustained high glucose decreased TE for Ins1, Ins2, Scgn, Slc2a2, Pfkfb3, Slc30a8, Vps41, Idh2, and Igf2, without affecting TE of Actb and Tubg1 (FIG. 6A). As observed in MIN6 cells, this led to significantly decreased steady-state protein abundance for SCGN, SLC2A2, PFKFB3, VPS41, IDH2, and IGF2 and a trend for decrease in SLC30A8 that did not reach significance (FIG. 6B). Collectively, the results in primary islets and in MIN6 cells confirm findings from ribosome profiling and provide evidence that translational regulation has a meaningful impact on the beta-cell proteome.

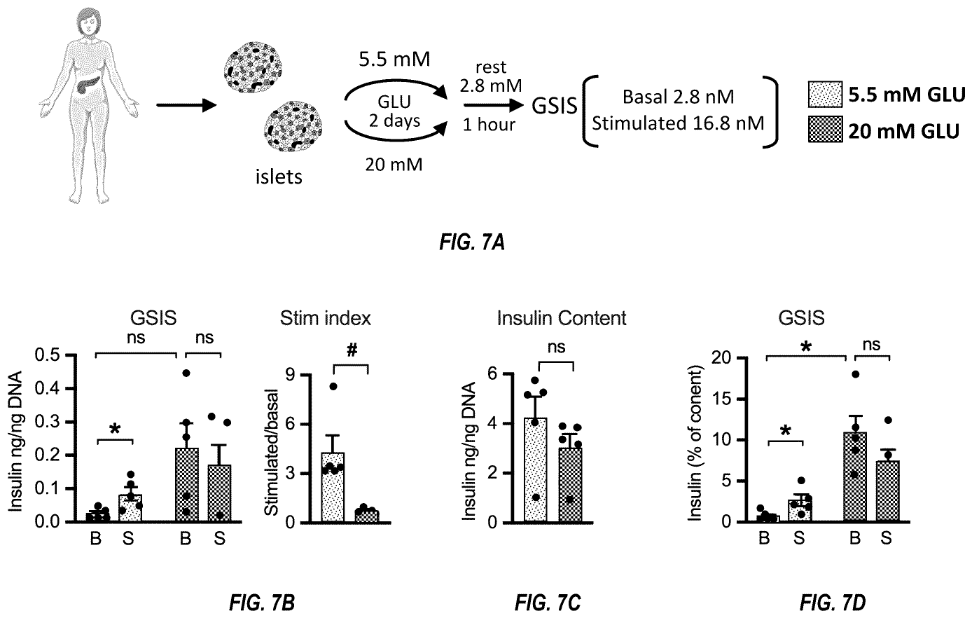

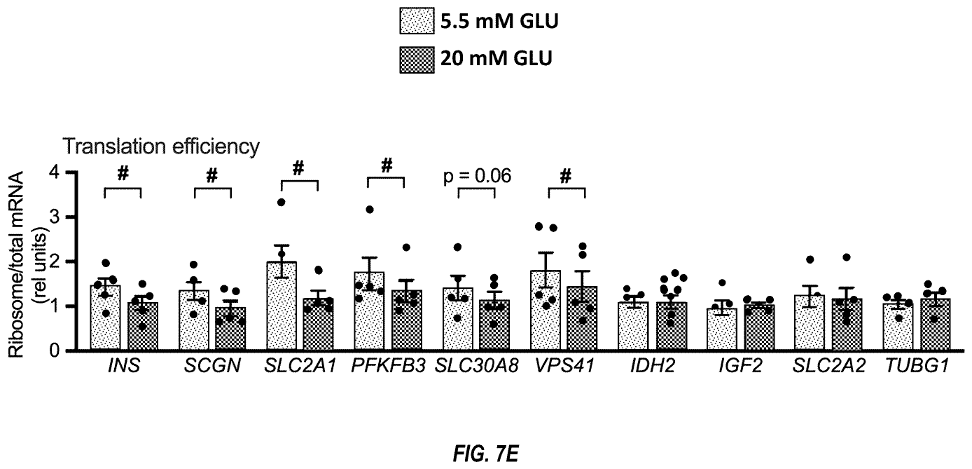

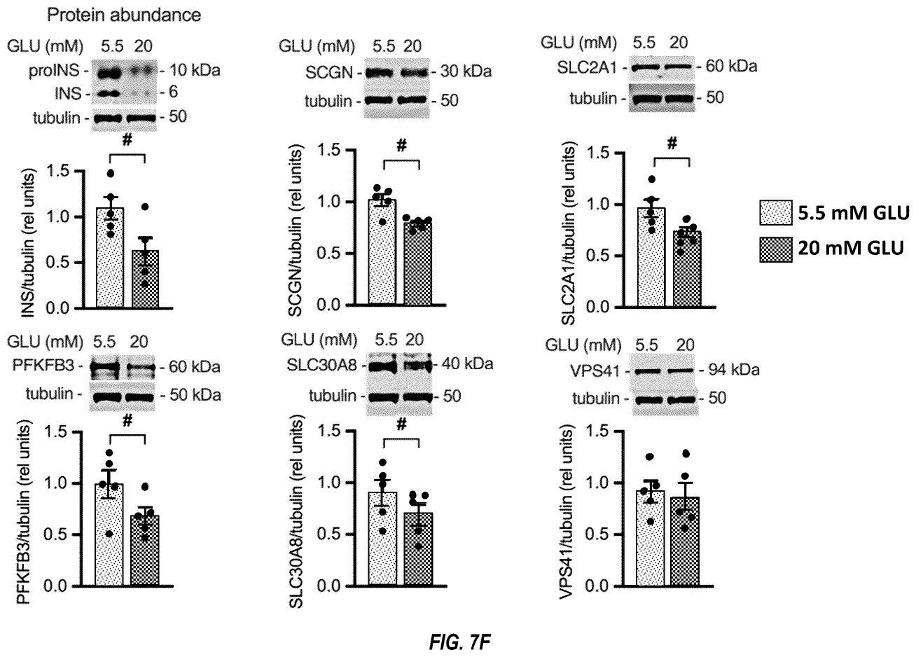

To investigate the clinical relevance of the observed glucose effects on translation, TE and protein abundance was analyzed following incubation of cadaveric human islets in 20 vs. 5.5 mM glucose (FIG. 7A). Exposure of human islets to high glucose for 2 days was sufficient to increase basal insulin secretion and decrease stimulation index (FIGS. 7B and 7D), consistent with previous reports (16), and decreased insulin content (FIG. 7C). TE, as assessed by ribosome-associated/total mRNA was decreased for Ins, Scgn, Pfkfb3, and Vps41 (FIG. 7E). For Slc30a8, trend for decreased TE was not statistically significant and TE for Idh2 and Igf2 were unchanged. Although translation of Slc2a2 was unchanged, TE for Slc2a1, the main plasma membrane glucose transporter in human islets (31), was decreased. Consistent with findings in rodent islets, decreased TE led to decreased steady-state protein abundance for INS, SCGN, SLC2A1, PFKFB3, and SLC30A8, and (FIG. 7F). VPS41 protein, however, was unchanged.

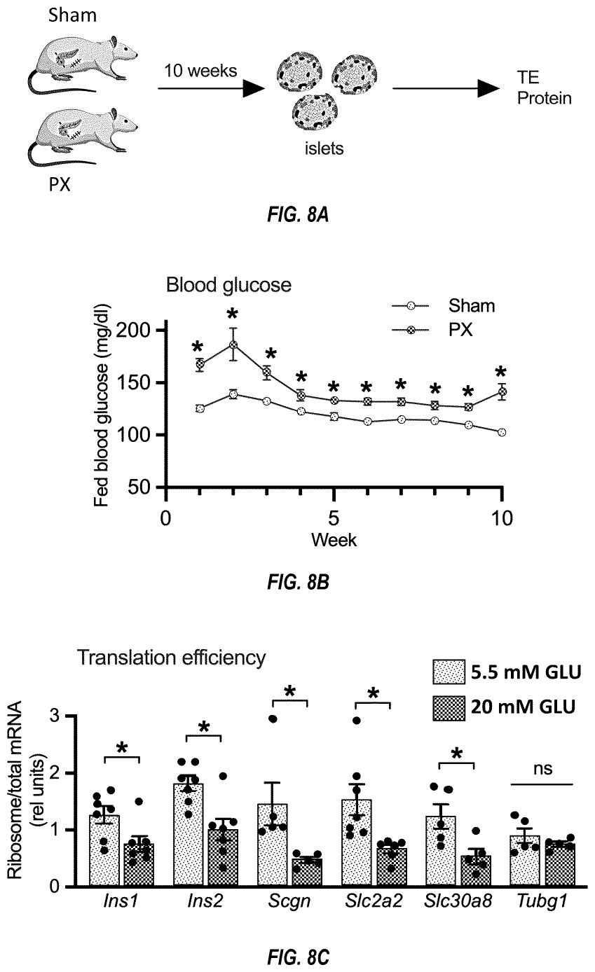

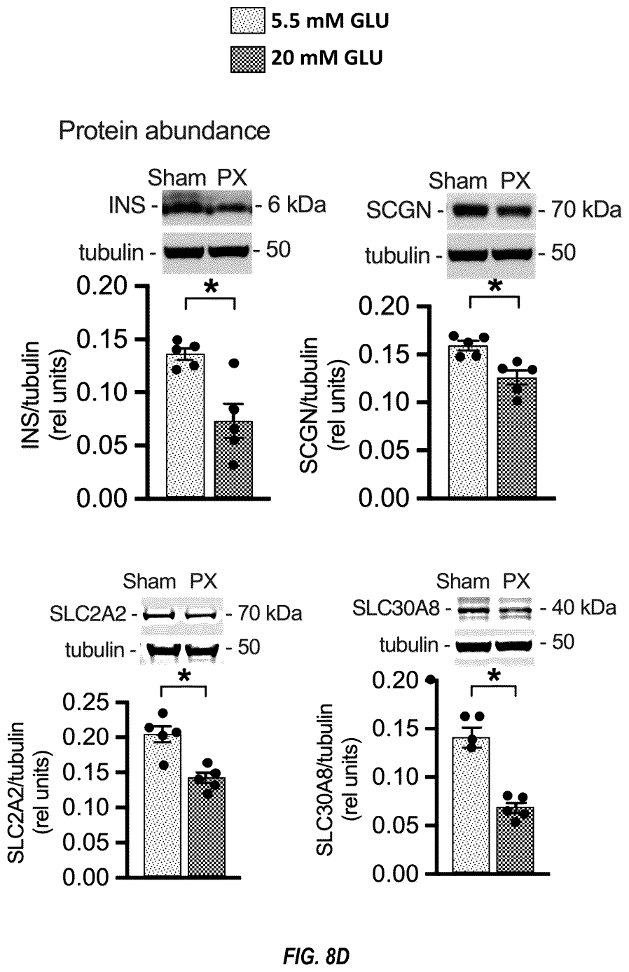

To extend these findings to an in vivo model of hyperglycemia, partial (90%) pancreatectomy (PX) or sham surgery was performed in adult male rats (FIG. 8A). Despite partial regeneration during the initial weeks of recovery, PX animals have sustained mild hyperglycemia and show selective loss of glucose-stimulated insulin secretion at 10 weeks post-surgery (32). As expected, PX rats had modest, but significantly elevated, fed blood glucose compared to sham animals (FIG. 8B). Islets isolated 10-weeks after PX demonstrated decreased TE for highly expressed genes including Ins1, Ins2, Scgn, Slc2a2, and Slc30a8 compared to sham with no effect on TE of Tubg1 control (FIG. 8C). For genes expressed at lower levels (Vps41, Idh2, Pfkfb3, and Igf2), recovery of mRNAs was insufficient to quantify TE. Steady-state protein levels were decreased for INS, SCGN SLC2A2, and SLC30A8 proteins (FIG. 8D). Thus, sustained exposure to high glucose in a pathophysiologically relevant setting suppressed translation of key mRNAs required for metabolism-coupled insulin secretion and abundance of their encoded proteins.

In summary, sustained high glucose selectively impairs mRNA translation of genes that serve critical roles at almost every step of glucose-metabolism coupled insulin secretion in pancreatic beta-cells. These nutrient-induced translation changes are coincident with impaired GSIS following prolonged exposure of cultured insulinoma cells or isolated islets ex vivo to high glucose and in the setting of 10 weeks of systemic hyperglycemia induced by partial pancreatectomy. These results show that programmatic dysregulation of beta-cell mRNA translation is a manifestation of glucose toxicity prior to the onset of ER stress or impairment of global translation. Translational downregulation decreases steady state levels of these proteins, which serve important roles in metabolism-coupled insulin secretion and optimal beta-cell function.

Beta-cells leverage structural and functional specializations for coupling glucose metabolism to robust insulin peptide production and secretion. The insulin mRNA is highly abundant, and ER and Golgi are extensive in beta-cells (33, 34). Moreover, translation of insulin and genes involved in insulin processing and secretory granule biogenesis is rapidly and coordinately upregulated when glucose is acutely increased from basal to stimulatory concentrations (8, 9). Recently, high throughput studies have shown that acute exposure to high glucose selectively upregulates translation of hundreds of beta-cell mRNAs (10, 11). The observations that mRNAs encoding proteins related to insulin processing, exocytosis, and glucose metabolism are enriched in polysomes and that production of these peptides is increased by acute glucose provide evidence that translation of functionally related proteins is coordinately regulated for optimal beta-cell function under physiological conditions. These data provide new evidence that concerted beta-cell translational regulation occurs in the pathophysiological setting of chronic high glucose exposure. Further, sustained high glucose conditions that impair GSIS are associated with translational downregulation of mRNAs required for metabolism-coupled insulin secretion. Moreover, these translational changes result in decreased protein abundance, which likely contributes to decreased secretory function.

Beta-cells synthesize up to a million proinsulin protein molecules per minute (35), creating a challenge for proper folding and processing of nascent proteins in the ER. Not surprisingly, prolonged exposure to high glucose can lead to ER-stress that activates the unfolded protein response to decrease total mRNA translation (36). The present experimental design incorporated ex vivo treatment of islets and MIN6 cells with glucose at concentrations and for durations that did not increase ER stress markers in order to model early nutrient-induced changes. Consistent with lack of engagement of the PERK-eIF2 alpha arm of ER-stress, total mRNA translation was unchanged under conditions in which translation of mRNAs involved in glucose-coupled insulin secretion was suppressed (37). It is also not surprising that ATF4 and JUND, proteins whose translation is increased under glucolipotoxic conditions that induce ER stress, were not upregulated in this study (17, 18). These results indicate that programmatic alterations in translation of specific mRNAs occurs prior to ER stress during the progression of beta-cell dysfunction.

Ribosome profiling has emerged as a powerful method for assessing mRNA translation in species ranging from yeast to human (38). However, TE calculated as the ratio of RPFs to RNA is an indirect measure of translation that could be confounded by increased RPFs resulting from translation pausing. The present strategy to also use nascent proteomics provided an approach to filter ribosome profiling results for translation changes that resulted specifically in synthesis of new proteins. As expected, only a fraction of newly synthesized proteins reflected altered TE, since nascent proteomics also includes changes in translation that result from increased mRNA abundance (39). These findings from MIN6 cell studies were validated in primary rat islets in which significant decreases in protein abundance for translationally down-regulated genes were demonstrated. In the rat partial pancreatectomy model, sustained mild hyperglycemia in vivo led to decreases in translation and protein abundance for the majority of these genes for which mRNA is highly abundant. Inability to quantify less abundant RNAs was likely a consequence of the present experimental design to analyze islets immediately upon isolation without overnight recovery of the islets in order to capture the impact of in vivo glycemia. Human islet analyses largely phenocopied observations in rodent studies with several notable exceptions. First, the observation that TE for Slc2a1, but not Slc2a2, was significantly decreased in human islets is likely attributable to differences in the glucose transporters utilized in these different species (Glut2/Slc2a2 in rodent and GLUT1/Slc2a1 in human beta-cells) (31). Second, absence of change in Idh2 and Igf2 translation in human islets may reflect species differences in regulation of insulin secretion, as their role has been best characterized in rodents (28, 30).

Regulatory steps following transcription play an important role in determining gene expression, and simultaneous RNA sequencing and proteomic analyses combined with metabolic labeling of macromolecules provides evidence that mRNA-specific translation rates are a major determinant of the cellular proteome (40). Moreover, the development of high throughput tools for discovery of coordinated mRNA-specific translation has advanced the understanding of how environmental cues shape gene expression. The present disclosure provides insights into nutrient-driven translational regulation that alters the abundance of proteins important for insulin secretion in settings of beta-cell dysfunction.

### Example 6. Sustained High Glucose Causes RPLP1 Dissociation from Actively Translating Ribosomes

To characterize the impact of sustained high glucose on the composition of actively translating ribosomes (polysomes) in beta-cells, quantitative TMT-labeled proteomics was used to analyze the stoichiometry of ribosomal proteins in polysomes isolated from MIN6 cells treated with 25 mM vs 5.5 mM glucose for 24 hours. Compared to other core ribosomal proteins, RPLP1 was reduced by 40% in ribosomes from high glucose-treated cells, whereas other core ribosomal proteins were unchanged (FIGS. 9A and 9B). Proteomic findings were confirmed in independent samples of high vs. low glucose-treated cells. Thus, exposure of pancreatic beta-cells to sustained high glucose, not only decreases translation of key genes for beta-cell function, but also dynamically remodels actively translating beta-cell ribosomes and decreases RPLP1 association with actively translating ribosomes.

RPLP1 is one of the acidic proteins that binds to periphery of the ribosome on the flexible P-stalk of the large ribosomal subunit. In contrast to most core ribosomal proteins that assemble co-transcriptionally onto ribosomal RNAs in the nucleolus and are degraded if not incorporated into nascent ribosomes, RPLP1 joins the nascent ribosome in late steps of biogenesis in the cytoplasm, where it is present in a soluble pool that exchanges with the ribosome-bound RPLP1 (Tsurugi K, Ogata K. (1985) J Biochem. 98(6):1427-31). Data herein suggest a model in which glucose toxicity causes remodeling of the ribosome structure that mediates mRNA-specific translational dysregulation in the setting of hyperglycemia.

To test whether altered stoichiometry of RPLP1 on polysomes reflects exchange of RPLP1 between ribosomes and a soluble pool, polysome and soluble fractions of MIN6 cells expressing amino-terminal FLAG-tagged RPLP1 were analyzed following treatment with 5.5 vs. 25 mM glucose for 24 hours. Sustained high glucose-induced decrease in polysome-associated FLAG-RPLP1 was associated with an increase in soluble FLAG-RPLP1 (FIGS. 10A and 10B). These findings support a model in which sustained high glucose stimulates RPLP1 exchange between the polysomes and a soluble pool.

### Example 7. Phosphorylated Amino Acid Residues of RPLP1 are Critical for Association with Translating Ribosomes

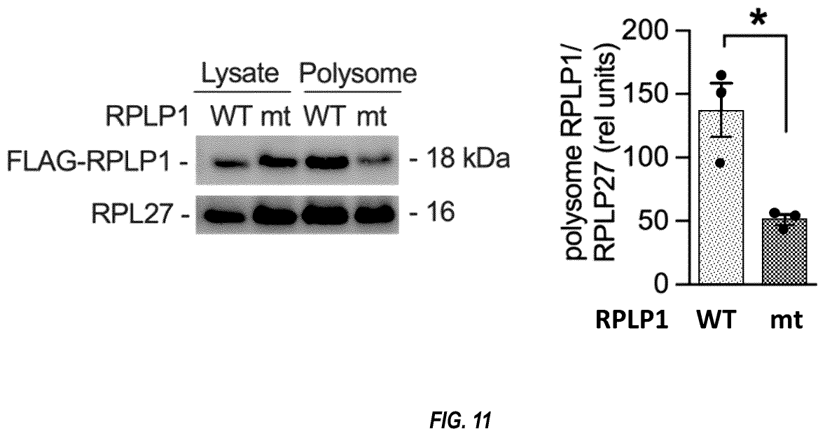

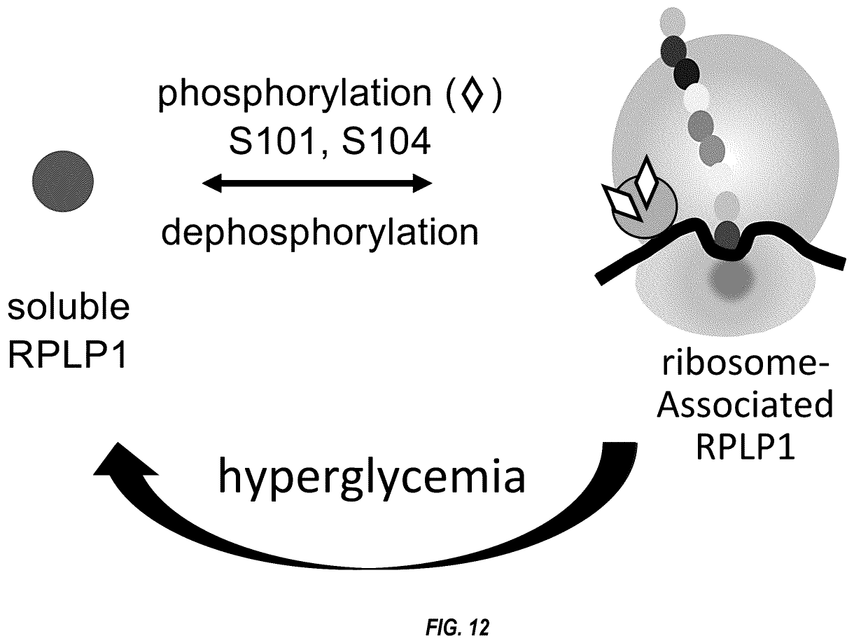

Phosphorylation of RPLP1 has been shown to regulate association of RPLP1 with the ribosome (MacConnell W P, Kaplan N O. (1982) J Biol Chem. 257(10):5359-66; Hasler P, et al. (1991) J Biol Chem. 266(21):13815-20), and phosphoproteomic analysis of MIN6 cells has shown that RPLP1 is phosphorylated near its carboxyl terminus on serine 101 and serine 104 (Sacco F, et al. (2016) Nat Commun. 7:13250). To test whether serine 101 and serine 104 are critical for RPLP1 ribosome association, wild type FLAG-RPLP1 and non-phosphorylatable mutant FLAG-RPLP1S101A/S104A were assessed for polysome association. The mutant demonstrated decreased association with polysomes (FIG. 11). These findings are consistent with a model in which phosphorylation at serine 101 and/or serine 104 promotes ribosome association, whereas conditions that cause dephosphorylation of these residues, such as hyperglycemia, cause RPLP1 to dissociate from ribosomes (FIG. 12).

There is growing evidence that heterogeneous ribosomes with varying stoichiometry of select ribosomal proteins (RPs) occur in different tissues or developmental contexts and direct functionally distinct programs of mRNA translation (Shi Z, et al. (2017) Mol Cell. 67(1):71-83; Segev N, et al. (2018) J Cell Biol. 217(1):117-126; Erratum in: J Cell Biol. (2018) 217(3):1155; Mageeney C M, et al. (2019) Mol Biol Cell. 30(17):2240-2253). To date, known examples of ribosome heterogeneity are stable characteristics of different cell types. The present disclosure provides new evidence that the composition of actively translating ribosomes in pancreatic beta-cells is dynamically modified by the nutrient environment.

The remodeling of beta-cell ribosomes under glucotoxic conditions that dysregulate mRNA-specific translation is likely to contribute to beta-cell dysfunction in the setting of hyperglycemia, because the proteins whose translation is altered are critical for glucose-stimulated insulin secretion. The finding that RPLP1 mutations that remove sites of phosphorylation similarly decrease RPLP1 ribosome association indicates that dephosphorylation of RPLP1 is a likely driver of its dissociation from ribosomes in the setting of nutrient excess. Further, regulation of post-translational modifications of RPLP1, such as by phosphorylating RPLP1, inhibiting dephosphorylation of RPLP1, and/or expressing an RPLP1 mutant comprising one or more phosphomimetic amino acids as described herein, may be able combat the effects of high glucose conditions on pancreatic beta-cells to prevent their dysfunction.

A variety of materials and methods were used to generate the data shown in FIGS. 1-11. Table 3 provides a list of reagents used in these studies.

TABLE 3
Reagents

| Item | Vendor | Catalog number |
|---|---|---|
| Direct-zol RNA Miniprep Kits, 50 preps | Zymo research | R2050 |
| DNA Clean & Concentrator-5 | Zymo research | D4003 |
| Novex TBE-Urea Gels, 15%, 12 well | Thermo Fisher | EC68852BOX |
| Novex ™ TBE Gels, 8%, 12 well | Thermo Fisher | EC62152BOX |
| ProtoScript II | New England Biolabs | M0368L |
| Dynabeads ™ MyOne ™ Streptavidin C1 | Thermo Fisher | 65001 |
| CircLigase ™ II ssDNA Ligase | Epicentre | CL9021K |
| T4 Polynucleotide Kinase | New England Biolabs | M0201S |
| T4 RNA Ligase 2, truncated K227Q | New England Biolabs | M0351S |
| 5′ DNA Adenylation Kit | New England Biolabs | E2610L |
| Phusion ® High-Fidelity DNA Polymerase | New England Biolabs | M0530S |
| 10 bp ladder (1 ug/ul) | New England Biolabs | N0364S |
| Turbo Dnase | Thermo Fisher | AM2239 |
| 100 mg/ml Cycloheximide | Sigma | C4859 |
| 1M Tris*Cl pH 8 RNase-free | Invitrogen | AM9855G |
| 5M NaCl RNase-free | Invitrogen | AM9760G |
| 1M MgCl2 RNase-free | Invitrogen | AM9530G |
| Triton X-100 molecular biology grade | Sigma | T8787 |
| SUPERase*In 20 U/ul | Invitrogen | AM2694 |
| Sucrose molecular biology grade | VWR | IB37160 |
| 3M NaOAc pH 5.5 RNase-free | Invitrogen | AM9740 |
| RNase I 10 U/ul | Thermo Fisher | EN0601 |
| Trizol | Thermo Fisher | 15596018 |
| 0.5M EDTA RNase-free | Invitrogen | AM9260G |
| miRNA marker | New England Biolabs | N2102S |
| 10x TBE RNase-free | Promega | V4251 |
| 10 mM dNTP mix | Invitrogen | 18427013 |
| SsoAdvanced Universal SYBR ® Green Supermix | Biorad | 1725270 |
| 20X SSC | Invitrogen | AM9770 |
| Ultra Sensitive Insulin ELISA kit | Crystal Chem | 90080 |
| O-propargyl-puromycin | Click Chemistry Tools | 1407-5 |
| Click-iT ™ Plus Alexa Fluor ™ 647 Picolyl | Fisher Scientific | C10643 |
| Azide Toolkit | | |
| TBTA | Click Chemistry Tools | 1061-500 |
| Biotin azide | Click Chemistry Tools | 1265-5 |
| BCA protein assay kit | Pierce | PI23225 |
| Biotin-alkyne | Click Chemistry Tools | TA105 |
| Pierce ™ High Capacity Streptavidin Agarose | Thermo Fisher Scientific | 20359 |
| Lipofectamine ™ transfection reagent | Thermo Fisher Scientific | 18324012 |
| Plus ™ reagent | Thermo Fisher Scientific | 11514015 |
| Polybrene^{R} | Santa Cruz Biotechnology | CAS 28728-55-4 |
| 25 mm 0.45 um Syringe Filter | Thermo Fisher Scientific | 723-2545 |
| Ready-to-use lentiviral plasmid packaging mix | Cellecta | CPCP-K2A |

### Rodent Islets

Islets were isolated from 7-8-week-old male Sprague-Dawley rats (Taconic Biosciences) by collagenase digestion followed by density gradient centrifugation as previously described (41). Islets were hand-picked and cultured overnight in RPMI 1640 containing 11 mM glucose, 1000 FCS, 100 units/ml penicillin, 100 μg/ml streptomycin at 37° C. with 5% CO₂. For analysis of GSIS, RNA or protein, media was changed to RPMI media containing either 5.5 mM or 16.7 mM glucose for 4 days.

### Human Islets

Islets from cadaveric nondiabetic donors (ages 30-50) (Table 4) were obtained from Prodo Labs and cultured overnight in RPMI 1640 containing 5.5 mM glucose, 10% FCS, 100 units/ml penicillin, 100 μg/ml streptomycin at 37° C. with 5% CO₂. The following day, islets were incubated in media containing either 5.5 mM or 16.7 mM glucose for 2 days prior to analysis of GSIS, RNA or protein.

TABLE 4
Human Islets from Prodo Labs

| | | | | | | Islet |
|---|---|---|---|---|---|---|
| Donor ID | Age | Sex | Race | HbA1c | BMI | purity |
| HP-23044-01 | 36 | Female | Caucasian | 4.90% | 34 | 85% |
| HP-23083-01 | 47 | Female | Hispanic | 5.40% | 30.6 | 90% |
| HP-23098-01 | 42 | Female | Hispanic | 5.20% | 29.3 | 90% |
| HP-23123-01 | 47 | Male | African | 5.70% | 28.4 | 90% |
| | | | American | | | |

### Cell Lines

Low passage MIN6 cells (generously provided by Dr. Jun-ichi Miyazaki) were cultured in DMEM containing 25 mM glucose, 15% FBS, 0.1 mM β-mercaptoethanol, 100 units/ml penicillin and 100 μg/ml streptomycin at 37° C. with 5% CO₂. For analyses of translation, MIN6 cells were incubated in DMEM media containing 5.5 mM or 25 mM glucose for 24 hours prior to analysis of GSIS, RNA or protein. (293T cells were obtained from ATCC CRL-3216).

### GSIS

Following incubations at different glucose concentrations, 10-12 islets (~150 μm diameter) in triplicate or MIN6 cells (105/35 mm well) in duplicate were washed and incubated with Krebs Ringer Bicarbonate HEPES buffer (KRBH: 137 mM NaCl, 4.8 mM KCl, 1.2 mM KH₂PO4, 1.2 mM MgSO₄·7H₂O, 2.5 mM CaCl₂·2H₂O, 5 mM NaHCO₃, 16 mM HEPES, 0.1% BSA) containing 2.8 mM glucose for 1 hour. Following media change, cells were successively incubated in KRBH containing 2.8 and 16.7 (islet) or 16.8 mM (MIN6) glucose, each for 1 hour. Media was collected for insulin quantification by Ultra Sensitive Mouse Insulin ELISA (Crystal Chem). Insulin was normalized to DNA content for islets (CyQuant cell proliferation kit, Fisher Scientific C7026) and to cell number for MIN6 cells.

RT-qPCR Quantification of mRNA

Total RNA was isolated from cell lysates, sucrose density gradient fractions, or ribosome pellets using Trizol or Trizol-LS reagents (Invitrogen) and Direct-zol RNA miniprep kit (Zymo Research). RNA recovered from sucrose density gradient fractions was treated with 600 units/ml heparinase (NEB P0735S, 20 minutes, room temperature [RT]). 500 ng to 1 μg RNA was reverse transcribed using iScript cDNA synthesis kit (Biorad). RT-qPCR was performed using SsoAdvanced Universal SYBR Green Supermix (Biorad). RNA abundance was calculated according to the AACT method relative to 18S rRNA. Primers are listed in Table 5.

TABLE 5
Primer Sequences

| | | | | |
|---|---|---|---|---|
| | SEQ | | SEQ | |
| | ID | | ID | |
| Gene Symbol | NO | Forward Primer | NO | Reverse Primer |

Mouse Primers (sequence 5′ to 3′)

| | | | | |
|---|---|---|---|---|
| Slc30a8 | 83 | ACTGATGCGGCTCATCTCTT | 42 | GATGCAAAGGACAGACAGCA |
| Scgn | 2 | TCTCTAAGGAAGGCCGCATA | 43 | TTGTGGTGGAGGAAAAGGTC |
| Vps41 | 3 | GATCACATCGATTGGCTCCT | 44 | TGCAGCCATGTCGTACTCTC |
| Slc2a2 | 4 | GTTGGAAGAGGAAGTCAGGGCA | 45 | ATCACGGAGACCTTCTGCTCAG |
| Idh2 | 5 | CCGTCTTCAGAGAGCCAATC | 46 | GAAATGGACTCGTCGGTGTT |
| Ins1 | 6 | AAGCCCCGGGGACCTTCAG | 47 | TCTCCAGCTGGTAGAGGGAGCAG |
| Ins2 | 7 | CCCGGGAGCAGGTGACCTT | 48 | TCTCCAGCTGGTAGAGGGAGCAG |
| Igf2 | 8 | GTCGATGTTGGTGCTTCTCA | 49 | AAGCAGCACTCTTCCACGAT |
| Actb | 9 | GGCTCCCAGCACCATGAA | 50 | GCCACCGATCCACAGAGAGT |
| Tubg1 | 10 | ACCTCCTGGAACGGCTAAAT | 51 | TTGATCTGGGAGAAGGATGG |
| 18S | 11 | ACCTGGTTGATCCTGCCA | 52 | GCCATTCGCAGTTTCACTGTA |
| MafA | 12 | ATCACTCTGCCCACCATCAC | 53 | TGACCTCCTCCTTGCTGAAG |
| Ucn3 | 13 | TTGCTTCTCGGCTTACCTGT | 54 | TGTGCACGTCACAGAGATGA |
| Pdx1 | 14 | GACCTTTCCCGAATGGAACC | 55 | GTTCCGCTGTGTAAGCACC |
| Pfkfb3 | 15 | GGCTGTTCTACGCTGCCTAC | 56 | AGGTCCCTTCTTGCATCCT |

Rat Primers (sequence 5′ to 3′)

| | | | | |
|---|---|---|---|---|
| Slc30a8 | 16 | TGCTGTCTGTCCTTTGCATC | 57 | TTGTGCATCCTTGTGGTTGT |
| Scgn | 17 | TCAAGCTCATTTGGATGCTG | 58 | AGATGTCATGGCCAACCATT |
| Vps41 | 18 | CTGGGGAACTTCCATCAAGA | 59 | AGTCTCTGGAAGTGGCTGGA |
| Slc2a2 | 19 | CAATTTCATCATCGCCCTCT | 60 | TGCAGCAATTTCGTCAAAAG |
| Idh2 | 20 | CAGTCTGACATCCTGGCTCA | 61 | AGATGCTGGCAATAGGGTTG |
| Ins1 | 21 | AGCGTGGCATTGTGGATCAG | 62 | TCAAAGGCTTTATTCATTGC |
| | | | | AGAGG |
| Ins2 | 22 | CAGCACCTTTGTGGTTCTCACTT | 63 | CTCCACCCAGCTCCAGTTGT |
| Igf2 | 23 | GTCGATGTTGGTGCTTCTCA | 64 | AAGCAGCACTCTTCCACGAT |
| Actb | 24 | AGCCATGTACGTAGCCATCC | 65 | CTCTCAGCTGTGGTGGTGAA |
| Tubg1 | 25 | CCATCCTTCTCCCAGATCAA | 66 | CTTGGGCTGTAGCAGTCTCC |
| 18S | 26 | CATTCGAACGTCTGCCCTAT | 67 | GTTTCTCAGGCTCCCTCTCC |
| MafA | 27 | TTCAGCAAGGAGGAGGTCAT | 68 | CCGCCAACTTCTCGTATTTC |
| Ucn3 | 28 | CTTCAGCTGCCTCAACACAG | 69 | GCTGTGCTTGGGATTGGTAT |
| Pdx1 | 29 | GAGGACCCGTACAGCCTACA | 70 | CGTTGTCCCGCTACTACGTT |
| Pfkfb3 | 30 | GAGGAGTATGCACTGCGTGA | 71 | GTGAAGTGGGCATTTCAGGT |

Human Primers (sequence 5′ to 3′)

| | | | | |
|---|---|---|---|---|
| Slc30a8 | 31 | CCTGTGGTTGTCATCGAAGCCT | 72 | CTCACATGCCAGGTACACTAGC |
| Scgn | 32 | GCCTCTAAAGATGGTCGCATTCG | 73 | AAACTCCACGCTGCTGTCCAGT |
| Vps41 | 33 | CTACATCAGTGGACTTGCACCTC | 74 | CAGAAAGTGGCTGGATGATGTCC |
| Slc2a2 | 34 | ATGTCAGTGGGACTTGTGCTGC | 75 | AACTCAGCCACCATGAACCAGG |
| Slc2a1 | 35 | TTGCAGGCTTCTCCAACTGGAC | 76 | CAGAACCAGGAGCACAGTGAAG |
| Idh2 | 36 | AGATGGCAGTGGTGTCAAGGAG | 77 | CTGGATGGCATACTGGAAGCAG |
| Ins | 37 | TGTCCTTCTGCCATGGCCCT | 78 | TTCACAAAGGCTGCGGCTGG |
| Igf2 | 38 | GATCGAACGCTCTGTGGCAGG | 79 | GCTCTGAGGACCCTTGGAGAAA |
| Tubg1 | 39 | CACTCAAGAGGCTGACGCAGAA | 80 | GGTTGATCTGGGAGAAGGATGG |
| 18S | 40 | GCAGAATCCACGCCAGTACAAG | 81 | GCTTGTTGTCCAGACCATTGGC |
| Pfkfb3 | 41 | GGCAGGAGAATGTGCTGGTCAT | 82 | CATAAGCGACAGGCGTCAGTTTC |

### Quantification of Nascent Peptides Using OPP

During the last 2 hours of incubations in different media, 20 μM O-propargyl-puromycin (OPP, Click Chemistry Tools) was added to the media. Cells were lysed with RIPA buffer (50 mM Tris pH 8, 150 mM NaCl, 0.5% sodium deoxycholate, 1% NP-40, 0.1% SDS) containing cOmplete EDTA-free protease inhibitor cocktail (Sigma). Protein was quantified using bicinchoninic acid assay (BCA, Pierce). For in-gel quantification of total nascent peptides, 100-300 μg protein were used for copper-catalyzed cycloaddition reactions using the Click-iT Plus Alexa Fluor 647 Picolyl Azide Toolkit (Fisher Scientific). Proteins were separated using NuPAGE 10% Bis-Tris gels and gels were fixed with 10% acetic acid, 50% methanol. Total nascent peptides were detected by in-gel imaging at 647 nm and total protein was detected by far red epi imaging after staining with 0.1% Coomassie brilliant blue (BioRad ChemiDoc MP Imaging System). For detection of nascent insulin and tubulin, 1 mg of cell lysate was used for cycloaddition reactions containing 5% SDS, 500 μM Biotin azide, 5 mM DTT, 0.5 mM TBTA, and 5 mM CuSO₄ (1.5 hours, RT). Biotinylated protein was precipitated using methanol/chloroform, re-suspended in RIPA buffer, and incubated overnight, 4° C. with high-capacity streptavidin agarose beads (Pierce). Beads were washed twice sequentially with RIPA, 1 M KCl, 0.1 M Na₂CO₃, 2 M Urea in 50 mM Hepes, RIPA and eluted with 2× Laemmli buffer. Proteins were separated using NuPAGE 10%, Bis-Tris gels and immunoblotting was used to detect nascent proteins.

### Immunoblotting

Islets were lysed by sonication in buffer containing 5 mM EDTA, 7 M urea, 2 M thiourea, 100 mM sodium fluoride, 100 mM pyrophosphate, 10 mM orthovanadate, 50 mM PMSF, 1 μg/ml aprotinin (Pierce 78432), and 1% Triton. MIN6 cells were lysed in RIPA buffer containing complete protease inhibitor cocktail. 10-20 μg protein was separated on 10% NuPAGE Bis-Tris gels, transferred to PVDF membranes, blocked with 5% BSA, and blotted for the indicated proteins. Alexa Fluor-coupled (Invitrogen) secondary antibodies or HRP-coupled secondary antibodies (Cell Signaling Technologies) and chemiluminescent substrates (Biorad) were used for detection with a ChemiDoc MP Imaging System (Biorad). For FR-stress controls, islets were treated with 1 μM thapsigargin for 6 hours and MIN6 cells were treated with 5 μg/mL tunicamycin for 3 hours. Antibodies and dilutions are listed in

TABLE 6
Antibodies for western blotting

| Antibody | Vendor | Catalog number | Lot number | Dilution |
|---|---|---|---|---|
| α-Insulin | Cell Signaling Technologies (CST) | 3014S | 10 | 1:1000 |
| α-Tubulin | Sigma | T6199 | 029M4842V | 1:5000 |
| α-pPERK | Cell Signaling Technologies (CST) | 3179L | 20 | 1:500 |
| α-PERK | Cell Signaling Technologies (CST) | 3192S | 11 | 1:500 |
| α-ATF4 | Cell Signaling Technologies (CST) | 11815S | 6 | 1:500 |
| α-SCGN | Novus Biologicals | NBP1-88219 | A106808 | 1:500 |
| α-IDH2 | Cell Signaling Technologies (CST) | 56439S | 2 | 1:500 |
| α-VPS41 | Santa Cruz Biotechnology | sc-377118 | a0719 | 1:100 |
| α-SLC2A2/GLUT2 | Proteintech | 20436-1-AP | 94131 | 1:500 |
| α-IGF2 | Cell Signaling Technologies (CST) | 40941S | 1 | 1:1000 |
| α-SLC30A8 | Proteintech | 16169-1-AP | 25550 | 1:500 |
| α-PFKFB3 | Cell Signaling Technologies (CST) | 13123 | 2 | 1:500 |
| α-RPLP1 | ProteinTech | 21636-1-AP | 00063366 | 1:1000 |
| α-RPLP0 | Abcam | ab192866 | 4 | 1:1000 |
| α-RPL18A | ProteinTech | 15751-1-AP | N/A | 1:1000 |
| α-RPL27 | OriGene | TA810903 | W001 | 1:1000 |
| α-FLAG | Sigma | F-3165 | N/A | 1:1000 |

### Ribosome Profiling

MIN6 cells incubated in media with 5.5 mM or 25 mM glucose for 24 hours were treated with 100 μg/ml of cycloheximide for 5 minutes. Ribosome profiling was performed as previously described (42) except that 1 U/20×10⁶ cells RNase 1 (Thermo Scientific) was used and rDNA depletion was performed using biotinylated rDNA sequences (38). Input RNA was extracted using TRIzol (Invitrogen) and Direct-zol RNA miniprep kit. RNA libraries were generated using polyA enrichment, and Kapa stranded mRNA Hyper Prep (Illumina). RPF and RNA libraries were sequenced using Illumina NS500 single-end 75 bp reads. Data analyses employed the XPRESSyourself pipeline (43). Briefly, trimmed reads were aligned to the genome (Ensembl release version 102) with the two-pass option that removes rRNA alignments and PCR duplicates and counts reads that map to the exons or truncated coding sequences of the longest transcripts of protein-coding genes. XPRESSpipe was used for quality control analyses (RPF coverage, length and periodicity) and to obtain normalized quantification of RNA, RPF counts and TE defined as ratio of RPF to RNA. Differential expression and differential TE were performed using DESeq2 (44). Pathway analysis was performed by testing over-representation of genes with differential TE in the Reactome gene sets from MSigDB using the pre-ranked CAMERA method in the limma package with the function cameraPR (45).

### Sucrose Density Gradient Fractionation of Polysomes

5-50% sucrose gradients were generated using a BioComp Gradient Master IP from 5% and 50% sucrose solutions in sucrose buffer (10 Mm Tris pH 7.2, 60 mM KCl, 10 mM MgCl₂, 1 mM DTT, and 0.1 mg/ml heparin). MIN6 cells were treated with 100 μg/ml of cycloheximide for 5 minutes. Cells were lysed with ribosome profiling lysis buffer and layered onto gradients. Following centrifugation (SW41T, 222,200×g, 3 hours, 4° C.), fractions were collected using a BR-188 Density Gradient Fractionation System (Brandel). RNA was extracted from combined polysome fractions with TRIzol LS, and cleanup used Direct-zol RNA miniprep kit.

### AHA Nascent Proteomics

MIN6 cells were incubated in media with 5.5 mM or 25 mM glucose for 24 hours. During the last 2.5 hours, cells were changed to methionine-free media for 30 minutes, washed with PBS and then incubated in methionine-free media containing 250 μM AHA for 2 hours. Cells were collected, lysed in RIPA buffer containing cOmplete EDTA-free protease inhibitor cocktail, and proteins were quantified by BCA. 2 mg protein per condition was reduced with 15 mM DTT (1 hour, RT), alkylated with 20 mM iodoacetamide (20 minutes, dark, RT), quenched with 10 mM DTT (15 minutes, dark, RT), precipitated using methanol/chloroform, and resuspended in 50 mM HEPES, 150 mM NaCl, 2% SDS pH 7.2. Copper-catalyzed cycloaddition of biotin was performed with 1 mg of protein by addition of 100 uM TBTA, 1 mM sodium ascorbate, 1 mM copper sulfate, 100 uM biotin-alkyne (2 hours, RT). Proteins were precipitated to remove excess biotin-alkyne, re-suspended in 2% SDS, 5 mM DTT, and diluted with RIPA buffer to final SDS to <0.5%. Samples were mixed with 10 ul of high-capacity streptavidin beads (overnight, RT) and then washed twice sequentially with RIPA, 1 M KCl, 0.1 M Na₂CO₃, 2 M Urea in 50 mM Hepes, RIPA, and PBS pH 7.4. Tryptic digest, TMT labeling, separation into 6 fractions and LC-MS3 analysis was performed as described (46). MS2 spectra were searched using the COMET algorithm against a Uniprot composite database derived from the mouse proteome, exogenous sequence, known contaminants, and reverse sequences. Peptide spectral matches were filtered to a 1% FDR using the target-decoy strategy combined with linear discriminant analysis. The proteins from the 6 runs were filtered to a <1% FDR. At least 2 unique peptides were required for identification, and proteins were quantified only from peptides with a summed SN threshold of >150. Protein intensity was log₂ transformed, missing values imputed, and data was normalized such that all samples had the same median abundance (47). Limma was used to perform linear modeling and moderated t-tests, with adjustment for surrogate variable analysis as previously described (48, 49).

### Polysome Proteomics

Proteins were isolated from combined polysome fractions, precipitated, digested with trypsin, TMT labeled, and fractionated prior to LC-MS3 analysis as described (An and Harper Nat Cell Biol 2018; 20(2):135-43). Alternatively, 10-20 μg protein was separated on 10% NuPAGE Bis-Tris gels, transferred to PVDF membranes, blocked with 5% BSA, and immunoblotted.

### Partial Pancreatectomy

Six-week-old Sprague-Dawley (~100 g) male rats underwent 90% pancreatectomy or sham surgery as previously described (32). Under anesthesia with ketamine/xylazine, pancreatic tissue was removed by gentle abrasion with cotton-tipped applicators, leaving a small remnant 1-2 mm from the common bile duct and extending to the first loop of the duodenum. For sham surgery, the pancreas was disengaged from the mesentery but not removed. Post-operatively, body weights and morning fed glucose values were measured weekly. 10 weeks following surgery, islets were isolated as above and immediately lysed for analysis of total and ribosome-associated RNA and protein expression analysis.

Isolation of Ribosome-Associated mRNA from Rat and Human Islets

Following ex vivo incubation of rat and human islets with low or high glucose or immediately following isolation of islets from sham and PX rats, islets were treated with 100 μg/ml of cycloheximide for 5 minutes, washed with ice-cold PBS, lysed with ribosome profiling buffer. One-third of the sample was collected for total RNA isolation and the remaining material was centrifuged through a 1M sucrose cushion to collect pelleted ribosome-associated mRNA (435,400×g, 1 hour, 4° C.). Total and ribosome-associated mRNA were isolated using TRIzol (Invitrogen) and 1-bromo-3-chloropropane (Sigma) using phase separation method.

### Lentiviral Expression of Epitope-Tagged Wild Type and Mutant RPLP1

Lentiviral plasmids containing FLAG-tagged RPLP1^{WT} or RPLP1^{S101/104A} were custom synthesized by VectorBuilder. 293T cells were transfected with 2 μg ready-to-use lentiviral packaging mix (Cellecta) and 2 μg lentiviral plasmids expressing FLAG-tagged RPLP1^{WT} or RPLP1^{S101/104A} using Lipofectamine™ and Plus™ reagent (ThermoFisher Scientific), according to standard manufacturer's protocol. Supernatant containing lentivirus were collected on day 2 and day 3 post-transfection and filtered using 0.45 μm Nalgene syringe filters (ThermoFisher Scientific). Transduction of MIN6 cells with lentivirus was done by incubating cells with viral supernatant and 0.8 μg/ml polybrene (Santa Cruz Biotechnology).

### Statistics

For biochemical, cell biological, and physiological experiments, analyses were performed using GraphPad Prism. Data are presented as means±SE. Statistical significance was determined by unpaired or paired 2-tailed t-tests, as described on figure legends and p value<0.05 was considered significant. For ribosome profiling and nascent proteomics, p-values were adjusted for multiple tests and FDR<0.1 (Benjamini-Hochberg method) was considered significant.

### REFERENCES

1. Halban P A, Polonsky K S, Bowden D W, Hawkins M A, Ling C, Mather K J, et al. beta-cell failure in type 2 diabetes: postulated mechanisms and prospects for prevention and treatment. Diabetes Care. 2014; 37(6):1751-8.
2. Evans-Molina C, Sims E K, DiMeglio L A, Ismail H M, Steck A K, Palmer J P, et al. beta-cell dysfunction exists more than 5 years before type 1 diabetes diagnosis. JCI Insight. 2018; 3(15).
3. Effect of intensive therapy on residual beta-cell function in patients with type 1 diabetes in the diabetes control and complications trial. A randomized, controlled trial. The Diabetes Control and Complications Trial Research Group. Ann Intern Med. 1998; 128(7):517-23.
4. Chen H S, Wu T E, Jap T S, Hsiao L C, Lee S H, and Lin H D. Beneficial effects of insulin on glycemic control and beta-cell function in newly diagnosed type 2 diabetes with severe hyperglycemia after short-term intensive insulin therapy. Diabetes Care. 2008; 31(10):1927-32.
5. Garvey W T, Olefsky J M, Griffin J, Hamman R F, and Kolterman O G. The effect of insulin treatment on insulin secretion and insulin action in type II diabetes mellitus. Diabetes. 1985; 34(3):222-34.
6. Itoh N, and Okamoto H. Translational control of proinsulin synthesis by glucose. Nature. 1980; 283(5742):100-2.
7. Wicksteed B, Uchizono Y, Alarcon C, McCuaig J F, Shalev A, and Rhodes C J. A cis-element in the 5′ untranslated region of the preproinsulin mRNA (ppIGE) is required for glucose regulation of proinsulin translation. Cell Metab. 2007; 5(3):221-7.
8. Guest P C, Bailyes E M, Rutherford N G, and Hutton J C. Insulin secretory granule biogenesis. Co-ordinate regulation of the biosynthesis of the majority of constituent proteins. Biochem J. 1991; 274 (Pt 1)(Pt 1):73-8.
9. Alarcon C, Lincoln B, and Rhodes C J. The biosynthesis of the subtilisin-related proprotein convertase PC3, but no that of the PC2 convertase, is regulated by glucose in parallel to proinsulin biosynthesis in rat pancreatic islets. J Biol Chem. 1993; 268(6):4276-80.
10. Zakaria A, Berthault C, Cosson B, Jung V, Guerrera I C, Rachdi L, et al. Glucose treatment of human pancreatic beta-cells enhances translation of mRNAs involved in energetics and insulin secretion. J Biol Chem. 2021; 297(1):100839.
11. Bulfoni M, Bouyioukos C, Zakaria A, Nigon F, Rapone R, Del Maestro L, et al. Glucose controls co-translation of structurally related mRNAs via the mTOR and eIF2 pathways in human pancreatic beta-cells. Front Endocrinol (Lausanne). 2022; 13:949097.
12. Schatz H, Nierle C, and Pfeiffer E F. (Pro-) insulin biosynthesis and release of newly synthesized (pro-) insulin from isolated islets of rat pancreas in the presence of amino acids and sulphonylureas. Eur J Clin Invest. 1975; 5(6):477-85.
13. Mittendorfer B, Patterson B W, Smith G I, Yoshino M, and Klein S. beta-cell function and plasma insulin clearance in people with obesity and different glycemic status. J Clin Invest. 2022; 132(3).
14. Eizirik D L, Korbutt G S, and Hellerstrom C. Prolonged exposure of human pancreatic islets to high glucose concentrations in vitro impairs the beta-cell function. J Clin Invest. 1992; 90(4):1263-8.
15. Ling Z, Kiekens R, Mahler T, Schuit F C, Pipeleers-Marichal M, Sener A, et al. Effects of chronically elevated glucose levels on the functional properties of rat pancreatic beta-cells. Diabetes. 1996; 45(12):1774-82.
16. Marselli L, Piron A, Suleiman M, Colli M L, Yi X, Khamis A, et al. Persistent or Transient Human beta-cell Dysfunction Induced by Metabolic Stress: Specific Signatures and Shared Gene Expression with Type 2 Diabetes. Cell Rep. 2020; 33(9):108466.
17. Good A L, Cannon C E, Haemmerle M W, Yang J, Stanescu D E, Doliba N M, et al. JUND regulates pancreatic beta-cell survival during metabolic stress. Mol Metab. 2019; 25:95-106.
18. Hatanaka M, Maier B, Sims E K, Templin A T, Kulkarni R N, Evans-Molina C, et al. Palmitate induces mRNA translation and increases ER protein load in islet beta-cells via activation of the mammalian target of rapamycin pathway. Diabetes. 2014; 63(10):3404-15.
19. Cnop M, Toivonen S, Igoillo-Esteve M, and Salpea P. Endoplasmic reticulum stress and eIF2alpha phosphorylation: The Achilles heel of pancreatic beta-cells. Mol Metab. 2017; 6(9):1024-39.
20. Shrestha N, De Franco E, Arvan P, and Cnop M. Pathological beta-Cell Endoplasmic Reticulum Stress in Type 2 Diabetes: Current Evidence. Front Endocrinol (Lausanne). 2021; 12:650158.
21. Miyazaki J, Araki K, Yamato E, Ikegami H, Asano T, Shibasaki Y, et al. Establishment of a pancreatic beta-cell line that retains glucose-inducible insulin secretion: special reference to expression of glucose transporter isoforms. Endocrinology. 1990; 127(1):126-32.
22. Guo H, Ingolia N T, Weissman J S, and Bartel D P. Mammalian microRNAs predominantly act to decrease target mRNA levels. Nature. 2010; 466(7308):835-40.
23. Yang S Y, Lee J J, Lee J H, Lee K, Oh S H, Lim Y M, et al. Secretagogin affects insulin secretion in pancreatic beta-cells by regulating actin dynamics and focal adhesion. Biochem J. 2016; 473(12):1791-803.
24. Kobayashi M, Yamato E, Tanabe K, Tashiro F, Miyazaki S, and Miyazaki J. Functional Analysis of Novel Candidate Regulators of Insulin Secretion in the MIN6 Mouse Pancreatic beta-cell Line. PLoS One. 2016; 11(3):e0151927.
25. WijesekaraN, Dai F F, Hardy A B, Giglou P R, Bhattacharjee A, Koshkin V, et al. Beta-cell-specific Znt8 deletion in mice causes marked defects in insulin processing, crystallisation and secretion. Diabetologia. 2010; 53(8):1656-68.
26. Burns C H, Yau B, Rodriguez A, Triplett J, Maslar D, An Y S, et al. Pancreatic beta-Cell-Specific Deletion of VPS41 Causes Diabetes Due to Defects in Insulin Secretion. Diabetes. 2021; 70(2):436-48.
27. Low B S J, Lim C S, Ding SSL, Tan Y S, Ng NHJ, Krishnan V G, et al. Decreased GLUT2 and glucose uptake contribute to insulin secretion defects in MODY3/HNF1A hiPSC-derived mutant beta-cells. Nat Commun. 2021; 12(1):3133.
28. Zhang G F, Jensen M V, Gray S M, El K, Wang Y, Lu D, et al. Reductive TCA cycle metabolism fuels glutamine- and glucose-stimulated insulin secretion. Cell Metab. 2021; 33(4):804-17 e5.
29. Arden C, Hampson L J, Huang G C, Shaw J A, Aldibbiat A, Holliman G, et al. A role for PFK-2/FBPase-2, as distinct from fructose 2,6-bisphosphate, in regulation of insulin secretion in pancreatic beta-cells. Biochem J. 2008; 411(1):41-51.
30. Modi H, Jacovetti C, Tarussio D, Metref S, Madsen O D, Zhang F P, et al. Autocrine Action of IGF2 Regulates Adult beta-Cell Mass and Function. Diabetes. 2015; 64(12):4148-57.
31. De Vos A, Heimberg H, Quartier E, Huypens P, Bouwens L, Pipeleers D, et al. Human and rat beta-cells differ in glucose transporter but not in glucokinase gene expression. J Clin Invest. 1995; 96(5):2489-95.
32. Bonner-Weir S, Trent D F, and Weir G C. Partial pancreatectomy in the rat and subsequent defect in glucose-induced insulin release. J Clin Invest. 1983; 71(6):1544-53.
33. Tillmar L, Carlsson C, and Welsh N. Control of insulin mRNA stability in rat pancreatic islets. Regulatory role of a 3′-untranslated region pyrimidine-rich sequence. J Biol Chem. 2002; 277(2):1099-106.
34. Dean P M. Ultrastructural morphometry of the pancreatic-cell. Diabetologia. 1973; 9(2):115-9.
35. Schuit F C, In't Veld P A, and Pipeleers D G. Glucose stimulates proinsulin biosynthesis by a dose-dependent recruitment of pancreatic beta-cells. Proc Natl Acad Sci USA. 1988; 85(11):3865-9.
36. Laybutt D R, Preston A M, Akerfeldt M C, Kench J G, Busch A K, Biankin A V, et al. Endoplasmic reticulum stress contributes to beta-cell apoptosis in type 2 diabetes. Diabetologia. 2007; 50(4):752-63.
37. Shi Y, Vattem K M, Sood R, An J, Liang J, Stramm L, et al. Identification and characterization of pancreatic eukaryotic initiation factor 2 alpha-subunit kinase, PEK, involved in translational control. Mol Cell Biol. 1998; 18(12):7499-509.
38. Ingolia N T, Brar G A, Rouskin S, McGeachy A M, and Weissman J S. The ribosome profiling strategy for monitoring translation in vivo by deep sequencing of ribosome-protected mRNA fragments. Nat Protoc. 2012; 7(8):1534-50.
39. Dieterich D C, Lee J J, Link A J, Graumann J, Tirrell D A, and Schuman E M. Labeling, detection and identification of newly synthesized proteomes with bioorthogonal non-canonical amino-acid tagging. Nat Protoc. 2007; 2(3):532-40.
40. Schwanhausser B, Busse D, Li N, Dittmar G, Schuchhardt J, Wolf J, et al. Global quantification of mammalian gene expression control. Nature. 2011; 473(7347):337-42.
41. Gotoh M, Maki T, Satomi S, Porter J, and Monaco A P. Effect of antilymphocyte serum on crude pancreatic islet allograft survival. Transplant Proc. 1987; 19(1 Pt 1):576-7.
42. McGlincy N J, and Ingolia N T. Transcriptome-wide measurement of translation by ribosome profiling. Methods. 2017; 126:112-29.
43. Berg J A, Belyeu J R, Morgan J T, Ouyang Y, Bott A J, Quinlan A R, et al. XPRESSyourself: Enhancing, standardizing, and automating ribosome profiling computational analyses yields improved insight into data. PLoS Comput Biol. 2020; 16(1):e1007625.
44. Love M I, Huber W, and Anders S. Moderated estimation of fold change and dispersion for RNA-seq data with DESeq2. Genome Biol. 2014; 15(12):550.
45. Wu D, and Smyth G K. Camera: a competitive gene set test accounting for inter-gene correlation. Nucleic Acids Res. 2012; 40(17):el33.
46. An H, Ordureau A, Komer M, Paulo J A, and Harper J W. Systematic quantitative analysis of ribosome inventory during nutrient stress. Nature. 2020; 583(7815):303-9.
47. Stekhoven D J, and Buhlmann P. MissForest—non-parametric missing value imputation for mixed-type data. Bioinformatics. 2012; 28(1):112-8.
48. Leek J T, and Storey J D. Capturing heterogeneity in gene expression studies by surrogate variable analysis. PLoS Genet. 2007; 3(9):1724-35.
49. Ritchie M E, Phipson B, Wu D, Hu Y, Law C W, Shi W, et al. limma powers differential expression analyses for RNA-sequencing and microarray studies. Nucleic Acids Res. 2015; 43(7):e47.
50. Sonenberg N and Hinnebusch A G. Regulation of translation initiation in eukaryotes: mechanisms and biological targets. Cell 2009; 136(4):731-45.
51. Genuth N R and Barna M. The Discovery of Ribosome Heterogeneity and Its Implications for Gene Regulation and Organismal Life. Mol Cell 2018; 71(3):364-74.
52. Kondrashov N, Pusic A, Stumpf C R, Shimizu K, Hsieh A C, Ishijima J, Shiroishi T, Bama M. Ribosome-mediated specificity in Hox mRNA translation and vertebrate tissue patterning. Cell 2011; 145(3):383-97.
53. Li H, Huo Y, He X, Yao L, Zhang H, Cui Y. A male germ-cell-specific ribosome controls male fertility. Nature. 2022; 612(7941):725-31.
54. Mageeney C M and Ware V C. Specialized eRpL22 paralogue-specific ribosomes regulate specific mRNA translation in spermatogenesis in Drosophila melanogaster. Mol Biol Cell 2019; 30(17):2240-53.
55. Segev N and Gerst J E. Specialized ribosomes and specific ribosomal protein paralogs control translation of mitochondrial proteins. Cell Bio. 2018; 217(1):117-26.
56. Shi Z, Fujii K, Kovary K M, Genuth N R, Rost H L, Teruel M N, Barna M. Heterogeneous Ribosomes Preferentially Translate Distinct Subpools of mRNAs Genome-wide. Mol Cell 2017; 67(1):71-83.
57. Peletski A A, Slavov N. Analyzing Ribosome Remodeling in Health and Disease. Proteomics 2020 September; 20(17-18): e2000039.
58. Harding H P, Ordonez A, Allen F, Parts L, Inglis A J, Williams R L, Ron D. The ribosomal P-stalk couples amino acid starvation to GCN2 activation in mammalian cells. Elife 2019; 8.
59. Inglis A J, Masson G R, Shao S, Perisic O, McLaughlin S H, Hegde R S, Williams R L. Activation of GCN2 by the ribosomal P-stalk. Proc Natl Acad Sci USA 2019; 116(11):4946-54.
60. Tsurugi K and Ogata K. Evidence for the exchangeability of acidic ribosomal proteins on cytoplasmic ribosomes in regenerating rat liver. J Biochem. 1985; 98(6):1427-31.
61. Sacco F, Humphrey S J, Cox J, Mischnik M, Schulte A, Klabunde T, et al. Nat Commun 2016; 7:13250.
62. Sanchez-Madrid F, Vidales F J, Ballesta J P. Effect of phosphorylation on the affinity of acidic proteins from Saccharomyces cerevisiae for the ribosomes. Eur J Biochem. 1981; 114(3):609-13.
63. Vidales F J, Robles M T, Ballesta J P. Biochemistry 1984; 23(2):390-6.
64. Zinker S. P5/P5′ the acidic ribosomal phosphoproteins from Saccharomyces cerevisiae. Biochim Biophys Acta 1980; 606(1):76-82.
65. MacConnell W P and Kaplan N O. The activity of the acidic phosphoproteins from the 80 S rat liver ribosome. J Biol Chem 1982; 257(10):5359-66.
66. Naranda T, Ballesta J P. Phosphorylation controls binding of acidic proteins to the ribosome. Proc Natl Acad Sci USA 1991; 88(23):10563-7.
67. Naranda T, Remacha M, Ballesta J P. The activity-controlling phosphorylation site is not the same in the four acidic ribosomal proteins from Saccharomyces cerevisiae. J Biol Chem 1993; 268(4):2451-7.
68. Doudna J A. The promise and challenge of therapeutic genome editing. Nature 2020; 578(7794):229-236.
69. Li G, Li X, Zhuang S, Wang L, Zhu Y, Chen Y, et al. Gene editing and its applications in biomedicine. Science China 65(4):660-700.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

1. A method of treating or preventing diabetes in a subject in need thereof comprising increasing the percentage of ribosome-associated RPLP1 or decreasing the percentage of soluble RPLP1 in a cell of the subject comprising:
a. phosphorylating RPLP1,
b. inhibiting dephosphorylation of RPLP1, and/or
c. expressing an RPLP1 mutant comprising one or more phosphomimetic amino acids,
thereby increasing the percentage of ribosome-associated RPLP1 and/or decreasing the percentage of soluble RPLP1 in the subject and treating or preventing diabetes.

2. The method of claim 1, wherein the subject has Type I (T1) or Type II (T2) diabetes or is at risk for T2 diabetes.

3. (canceled)

4. The method of claim 1, wherein increasing the percentage of ribosomes containing RPLP1 increases translation of one or more proteins encoded by a gene selected from INS, SCGN, IDH2, VPS41, SLC2A1, IGF2, SLC30A8, and PFKFB3.

5. The method of claim 1, wherein the method comprises administering to the subject a kinase activator or phosphatase inhibitor that phosphorylates RPLP1 and/or dephosphorylates RPLP1.

6. The method of claim 1, wherein the RPLP1 phosphorylation is at serine 101 and/or serine 104.

7. The method of claim 1, wherein expressing an RPLP1 mutant comprising one or more phosphomimetic amino acid mutations is performed by gene editing of RPLP1.

8. (canceled)

9. The method of claim 7, wherein the gene editing is performed with a system for gene editing, and wherein the system for gene editing comprises a CRISPR/Cas9 system, zinc-finger nuclease, transcription activator-like effector nuclease (TALEN), meganuclease, or group one intron encoded endonuclease (GIIEE).

10. The method of claim 7, wherein the RPLP1 mutant comprises one or more phosphomimetic amino acid mutations at an amino acid that is a serine in wildtype RPLP1.

11. The method of claim 10, wherein the serine in wildtype RPLP1 is serine 101 and/or serine 104.

12. The method of claim 1, wherein the one or more phosphomimetic amino acid mutations is an aspartic acid or glutamic acid.

13. The method of claim 1, wherein the cell is a pancreatic beta-cell.

14. The method of claim 1, wherein the method increases the levels of insulin within or released by the cell.

15. (canceled)

16. The method of claim 7, wherein:
a. the cell is an iPSC or pancreatic beta-cell in culture and the gene editing is performed in vitro, and the cell is introduced into the subject after the gene editing; or
b. the cell is an iPSC or pancreatic beta-cell in a subject and the gene editing is performed in vivo using a delivery system that selectively delivers a gene editing system to the iPSC or pancreatic beta-cell.

17. The method of claim 16, wherein insulin levels in the subject are increased and/or glucose levels in the subject are decreased.

18. A method of treating or preventing diabetes in a subject in need thereof comprising:
a. preparing iPSCs and/or pancreatic-beta-cells in vitro;
b. treating said iPSCs or pancreatic beta-cells with an agent for gene editing of RPLP1, wherein the gene editing causes expression of an RPLP1 mutant comprising one or more phosphomimetic amino acids in the cell; and
c. transplanting the treated iPSCs or pancreatic beta-cells into the subject, wherein the transplanting increases insulin levels in the subject and/or decreases glucose levels in the subject.

19. A method of reducing the incidence of hyperglycemia or immune attack on pancreatic beta-cells in a subject in need thereof comprising:
a. preparing iPSCs and/or pancreatic-beta-cells in vitro;
b. treating said iPSCs or pancreatic beta-cells with an agent for gene editing of RPLP1, wherein the gene editing causes expression of an RPLP1 mutant comprising one or more phosphomimetic amino acids in the cell; and
c. transplanting the treated iPSCs or pancreatic beta-cells into the subject, wherein the transplanting increases insulin levels in the subject and/or decreases glucose levels in the subject.

20. The method of claim 18, wherein the RPLP1 mutant comprises one or more phosphomimetic amino acid mutations at an amino acid that is a serine in wildtype RPLP1.

21. The method of claim 20, wherein the serine in wildtype RPLP1 is serine 101 and/or serine 104.

22. The method of claim 21, wherein the one or more phosphomimetic amino acid mutations is an aspartic acid or glutamic acid.

23. (canceled)

24. (canceled)

25. (canceled)

26. (canceled)
